(19) United States
(12) Patent Application Publication
(10) Pub. No.: US 2021/0311028 A1
(43) Pub. Date: Oct. 7, 2021

(54) ANTIBODY OR APTAMER CONJUGATED-LIPID VESICLES AND DETECTION METHODS AND MICROFLUIDICS DEVICES USING SAME

(71) Applicant: SENSOR-KINESIS CORPORATION, Los Angeles, CA (US)

(72) Inventor: Roger D. Kornberg, Atherton, CA (US)

(73) Assignee: Autonomous Medical Devices Inc., Inglewood, CA (US)

(21) Appl. No.: 17/266,503

(22) PCT Filed: Oct. 11, 2019
(86) PCT No.: PCT/US2019/055993

§ 371 Date: Feb. 5, 2021

Related U.S. Application Data
(60) Provisional application No. 62/744,895, filed on Oct. 12, 2018.
(60) Provisional application No. 62/886,759, filed on Aug. 14, 2019.

(51) Int. Cl. G01N 33/532; B01L 3/00; G01N 33/543

(52) U.S. Cl. CPC G01N 33/532; B01L 3/502715; B01L 2300/0636; B01L 2200/10; G01N 33/54386

(57) ABSTRACT

The disclosure relates to antibody conjugates or aptamer conjugates comprising an antibody linked to a lipid vesicle comprising a detectable label, methods for detecting a marker or several markers in a sample by using said antibody and aptamer conjugates. The present disclosure further relates to microfluidics devices to detect one or more markers in a sample by using said antibody and aptamer conjugates.

### CROSS-REFERENCE TO RELATED APPLICATIONS

This application claims priority from United States Provisional Applications No. 62/744,953, filed Oct. 12, 2018 and No. 62/886,759, filed Aug. 14, 2019, the contents of which are hereby incorporated by reference in their entirety.

### BACKGROUND OF THE INVENTION

Markers, such as biomarkers are tools for the diagnosis, monitoring, and screening for a number of diseases. Biomarkers can also be used to detect disease risk factors allowing the physician to recommend or prescribe more intensive monitoring or testing of a patient. In many cases it is possible to diagnose diseases, such as cancer or rheumatic diseases, via determining concentrations of specific biomarkers in blood, i.e., a marker profile. For such applications based on the detection of multiple biomarkers or one of a plurality of biomarkers in one sample, a cost-effective and rapid analysis system with small sample consumption is required.

The two hallmarks of a diagnostic biomarker analysis system are sensitivity and specificity. Sensitivity refers to the percentage of patients with a disease who will test positive in the assay. False negative results dilute the sensitivity of an assay. Specificity refers to the percentage of patients without disease who test as negative in the assay. False positive results dilute the specificity of a diagnostic assay.

Although both are extremely important, low sensitivity in a diagnostic assay for cancer can be life threatening if false negative results prevent individuals with cancer from receiving timely treatment.

Microbial and viral identification usually rely on conventional approaches of plating and culture methods, as well as on biochemical testing, microscopy, etc.

Over the last 20 years, many new methods have been developed, including immunological methods, polymerase chain reaction (PCR) and biosensors (Deisingh, A. K.; Thompson, M. Biosensors for the detection of bacteria. Can. J. Microbiol. 2004; 50, 69-77). Plating and culture methods often fail to provide the required specificity and sensitivity and can take up to 7 days to complete. PCR, although very specific and suitable for screening purposes, still fails to produce accurate results when enumeration of viable cells is needed (March, C. et al. J. Immunol. Methods 2005, 303, 92-104). Immunological detection with antibodies is perhaps the most successful technology employed for the detection of cells, spores, viruses and toxins alike (Iqbal, S. S. et al. Biosens. Bioelectron. 2000, 15, 549-578). The availability of monoclonal antibodies, together with the emergence of recombinant antibody phage display technology, has made immunological detection of microbial contamination more sensitive, specific, reproducible and reliable. These technologies, when incorporated in biosensors, significantly shorten the assay time and improve the analytical performance of pathogen detection.

Enzyme-linked immunosorbent assays (ELISA) and radioimmunoassays are generally regarded as the gold standards for the detection of markers or microorganisms, in terms of sensitivity and selectivity, and a number of research groups are directing efforts towards implementing such assays with microfabricated devices.

Liposomes are micron-sized spherical shells of amphipathic molecules which isolate an interior aqueous space from the bulk exterior aqueous environment. They can be made to contain hydrophobic molecules within their membrane, or hydrophilic markers within their internal aqueous space, or both. Because of this versatility, liposomes are of interest both as potential vehicles for the delivery of drugs in vivo and as the basis for immunoassay systems in vitro.

Liposome lysis can be detected in a variety of ways and depends upon the nature of the marker initially encapsulated within the liposome. Kataoka, et al., (Eur. J. Biochem., 24:123 (1971)), for example, describe Lipid A sensitized liposomes which release a spectrophotometrically detectable glucose marker when incubated with an anti-Lipid A anti-serum and complement source. Yet another means for detecting lysis involves initially encapsulating within the liposome a fluorophore at self-quenching concentrations. Upon liposome lysis, dilution of the fluorophore occurs, re-establishing fluorescence. The increase in fluorescence is proportional to the amount of analyte present in the sample. Ishimori, et al., (J. Immuno. Methods, 75:351-360 (1984)) describe an immunoassay technique using immunolysis of liposomes to measure antibody against protein antigens such as human IgG. The marker used was carboxyfluorescein, and the technique was reported to be effective at detecting 10⁻¹⁵ moles of anti-human IgG antibody or human IgG.

Antibody conjugates are widely used as diagnostics and imaging reagents. However, many such conjugates lose in sensitivity and specificity due to nonspecific labeling techniques. The ability to detect very rare cells or markers at low concentrations in the blood with accuracy and sensitivity is still a significant problem for molecular diagnostics. Typical protein detection methods ELISAs are typically not sensitive enough to detect low concentrations of important biological markers such as troponin, prostate-specific antigen, or viral coat proteins.

There is a need for convenient and portable methods and devices for the detection of markers or microorganisms, including biomarkers and environmental markers, especially more sensitive, specific, and robust sensors. See, e.g., Kaisti, M. Biosensors and Bioelectronics, 2017, vol. 98:437-448, incorporated by reference herein in its entirety. Interactions involving macromolecules, such as antibodies, occur relatively slowly, on the order of 10⁵ specific binding events per second. By contrast, binding of ions to counter ions occurs much more rapidly, on the order of 10¹⁰ or more events per second. The detection of ions in solution, however, is complicated by the screening of detectors from such molecules by oppositely charged ions and other unrelated ions in the solution. See, e.g., Kaisti, M. Biosensors and Bioelectronics, 2017, vol. 98:437-448, incorporated by reference herein in its entirety. Accordingly, there is a need in the art for improving the selective, sensitive and robust detection of markers or microorganisms, maintaining a high specificity.

### SUMMARY OF THE INVENTION

The present disclosure provides a binding member-lipid vesicle conjugate, which can be used to detect a marker or several markers in a sample. The present disclosure also provides methods for rapidly detecting markers including markers at low concentrations, while maintaining high sensitivity and specificity with a very short assay time. The present method provides an extraordinary sensitivity to detect low concentrations of markers in samples.

A first aspect of the present disclosure provides an antibody conjugate comprising an antibody linked to an amphiphilic lipid vesicle, wherein the vesicle comprises a detectable label. This aspect of the disclosure also provides an aptamer conjugate comprising an aptamer linked to an amphiphilic lipid vesicle, wherein the vesicle comprises a detectable label.

In some embodiments, the amphiphilic lipid is a phospholipid. The phospholipid may be a phosphatidic acid, a phosphatidylethanolamine, a phosphatidylcholine, a phosphatidylserine, a phosphatidylglycerol, a phosphatidylinositol. Optionally, the phospholipid is a phosphatidylcholine, such as lecithin.

In some embodiments, the lipid vesicle membrane is uniform. In some embodiments, the lipid vesicle membrane comprises one lipid bilayer. Optionally, the lipid vesicle comprises a bilayer. In some embodiments, the lipid vesicle is detergent-soluble. Optionally, the detergent is a non-ionic detergent. In some embodiments, the lipid vesicle is susceptible to enzymatic disruption.

In some embodiments, at least 95% of the detectable label remains in the vesicle for six months. The detectable label may be capable of being detected by a surface acoustic wave device. Optionally, the detectable label is capable of being detected by a surface acoustic wave device is selected from the group of a magnetic particle, a large metal particle and a spore. In some embodiments, the detectable label is capable of being detected by a field effect transistor. Optionally, the detectable label capable of being detected by a field effect transistor is selected from the group of a magnetic particle, a large metal particle and an ionic solution. The detectable label may be a fluorescent label, a radioactive label, an enzymatic label, a colorimetric substrate or a fluorogenic substrate.

In some embodiments, the antibody or the aptamer is linked to the lipid vesicle by a linker. Optionally, the peptide linker comprises a protease cleavage site. The linker may be released by cleavage of a disulfide bond.

A second aspect of the present disclosure provides a method for detecting a marker in a sample, the method comprising:
(a) contacting the sample with a capture molecule that binds the marker, wherein the capture molecule is affixed to a scaffold or is capable of being affixed to a scaffold,
(b) contacting the marker with a composition comprising an antibody conjugate according to the first aspect of the disclosure, wherein the antibody conjugate binds to a different epitope on the marker than the capture molecule;
(c) contacting the marker-bound antibody conjugate with conditions or a composition capable of releasing the detectable label from the amphiphilic lipid vesicle on the antibody conjugate; and
(d) detecting the detectable label.

This aspect of the disclosure also provides a method for detecting a marker in a sample using an aptamer conjugate that binds the marker, wherein the aptamer conjugate is an aptamer conjugate according to the first aspect of the disclosure.

In some embodiments, the scaffold is a detector for the detectable label. Optionally, the scaffold is adjacent to a detector for the detectable label. The capture molecule may be bound to a magnetic bead or a metallic bead, wherein the capture molecule binds to the scaffold upon the cycling of an electric current. In some embodiments, the detection step comprises the step of transporting the detectable label to a detector for the detectable label. In some embodiments the capture molecule is a capture antibody. Optionally, the capture molecule is a capture aptamer.

The method may further comprise the step of removing the unbound marker. In other embodiments, the method further comprises one or more steps of washing the capture molecule-bound marker. Optionally, the method further comprises one or more steps of washing the capture molecule-bound marker prior to contacting the capture molecule-bound marker with the antibody conjugate or the aptamer conjugate. The method may further comprise the step of removing the unbound antibody conjugate or the unbound aptamer conjugate. In some embodiments, the method further comprises one or more steps of washing the marker-bound antibody conjugate or the marker-bound aptamer conjugate before the releasing step.

In some embodiments, the composition capable of releasing the detectable label comprises a detergent. Optionally, the detergent is a non-ionic detergent. The composition capable of releasing the detectable label may comprise an enzyme.

In some embodiments, the detectable label is capable of being detected by a surface acoustic wave device. Optionally, the detectable label capable of being detected by a surface acoustic wave device is selected from the group consisting of a magnetic particle, a large metal particle and a spore.

In some embodiments, the detectable label is capable of being detected by a field effect transistor. Optionally, the detectable label capable of being detected by a field effect transistor is selected from the group consisting of a magnetic particle, a large metal particle and an ionic solution.

In some embodiments, the detectable label is selected from the group consisting of a fluorescent label, an enzymatic label, a radioactive label, a fluorogenic substrate and a colorimetric substrate.

In some embodiments, the method is performed on a microfluidics device. The detection of the marker may be possible or improved as a result of signal amplification. In some embodiments, the marker is a biomarker, an environmental marker, an allergen, or a microorganism. Optionally, the microorganism is selected from the group consisting of a bacterium, a fungus, an archaeon, an alga, a protozoan and a virus. In some embodiments, the sample is an environmental sample, a food sample, or a sample obtained from a subject.

A third aspect of the present disclosure provides a method of detecting one of a plurality of markers in a sample, the method comprising:
(a) contacting the sample with a first capture molecule and a second capture molecule, wherein the first capture molecule is affixed to a first scaffold or is capable of being affixed to the first scaffold and binds a first marker, wherein the second capture molecule is affixed to a second scaffold or is capable of being affixed to the second scaffold and binds a second marker, wherein the first marker is different from the second marker;
(b) contacting the first marker with a composition comprising a first antibody conjugate, wherein the first antibody conjugate is an antibody conjugate according to the first aspect of the disclosure, and wherein the first antibody conjugate recognizes a different epitope on the first marker than the first capture molecule;
(c) contacting the second marker with a composition comprising a second antibody conjugate, wherein the second antibody conjugate is an antibody conjugate according to the first aspect of the invention, and wherein the second antibody conjugate recognizes a different epitope on the second marker than the second capture molecule;
(d) contacting the first marker-bound first antibody conjugate with a composition capable of releasing a first detectable label from the amphiphilic lipid vesicle on the first antibody conjugate;
(e) contacting the second marker-bound second antibody conjugate with a composition capable of releasing a second detectable label from the amphiphilic lipid vesicle on the second antibody conjugate;
(f) performing a first detection step to detect the first detectable label; and
(g) performing a second detection step to detect the second detectable label.

In some embodiments, the first marker is contacted with the first capture molecule before the first marker is contacted with the first antibody conjugate. Optionally, the first marker is contacted with the first capture molecule after the first marker is contacted with the first antibody conjugate. In some embodiments, the second marker is contacted with the second capture molecule before the second marker is contacted with the second antibody conjugate. Optionally, the second marker is contacted with the second capture molecule after the second marker is contacted with the second antibody conjugate. This aspect of the disclosure also provides a method of detecting one of a plurality of markers in a sample using a first aptamer conjugate that binds to a first marker and comprises a first detectable label and a second aptamer conjugate that binds to a second marker and comprises a second detectable label, wherein the first and second aptamer conjugates are aptamer conjugates according to the first aspect of the disclosure. This aspect of the disclosure further provides a method of detecting one of a plurality of markers in a sample using an antibody conjugate that binds to a first marker and comprises a first detectable label and an aptamer conjugate that binds to a second marker and comprises a second detectable label, wherein the antibody conjugate and the aptamer conjugate are an antibody conjugate and an aptamer conjugate according to the first aspect of the disclosure.

In some embodiments, the first scaffold is a detector for the first detectable label. In other embodiments, the first scaffold is adjacent to a detector for the first detectable label. In some embodiments, the first capture molecule is bound to a magnetic bead or a metallic bead, wherein the first capture molecule binds to the first scaffold upon the cycling of an electric current or a magnetic field. In some embodiments, the first capture molecule is a capture antibody. Optionally, the first capture molecule is a capture aptamer.

In some embodiments, the second scaffold is a detector for the second detectable label. In other embodiments, the second scaffold is adjacent to a detector for the second detectable label. In some embodiments, the second capture molecule is bound to a magnetic bead or a metallic bead, wherein the second capture molecule binds to the second scaffold upon the cycling of an electric current or a magnetic field. In some embodiments, the second capture molecule is a capture antibody. Optionally, the second capture molecule is a capture aptamer. Optionally, the first and second scaffolds are the same. In other embodiments, the first and second scaffolds are different. In some embodiments, the first detection step comprises the step of transporting the first detectable label to a detector for the first detectable label. Optionally, the second detection step comprises the step of transporting the second detectable label to a detector for the second detectable label.

The method may further comprise the step of removing the unbound first antibody conjugate or the unbound first aptamer conjugate. In some embodiments, the method of the third aspect further comprises the step of washing the marker-bound first antibody conjugate or the marker-bound first aptamer conjugate before releasing the first detectable label. The method may further comprise the step of removing the unbound second antibody conjugate or the unbound second aptamer conjugate. In some embodiments, the method further comprises the step of washing the marker-bound second antibody conjugate or the marker-bound second aptamer conjugate before releasing the second detectable label. Optionally, the first detectable label and the second detectable label are different, and the marker is detected by which detectable label is present. In other embodiments, the first detectable label and the second detectable label are the same, and the marker is detected by whether the detectable label is present in the first detection step or the second detection step. The first and second detection steps may be performed at sequentially. The first and second detection steps may be performed simultaneously at different locations.

The method may further comprise the step of removing the unbound first marker. In some embodiments, the method according to the third aspect further comprises the step of washing the capture molecule-bound first marker. Optionally, the method comprises the step of washing the capture molecule-bound first marker prior to contacting the capture molecule-bound first marker with the first antibody conjugate or the first aptamer conjugate. The method may further comprise the step of removing the unbound second marker. Optionally, the method further comprises the step of washing the capture molecule-bound second marker. In some embodiments, the method further comprises the step of washing the capture molecule-bound second marker prior to contacting the capture molecule-bound second marker with the second antibody conjugate or the second aptamer conjugate. In some embodiments, the method further comprises the step of washing the detector prior to the first detection step. The method may further comprise the step of washing the detector prior to the second detection step.

In some embodiments, the composition capable of releasing the first detectable label comprises a detergent. Optionally, the detergent is a non-ionic detergent. In some embodiments, the composition capable of releasing the first detectable label comprises an enzyme. Optionally, the enzyme is Phospholipase A2.

In some embodiments, the composition capable of releasing the second detectable label comprises a detergent. Optionally, the detergent is a non-ionic detergent. In some embodiments, the composition capable of releasing the second detectable label comprises an enzyme. Optionally, the enzyme is Phospholipase A2.

In some embodiments, the composition capable of releasing the first detectable label is the same as the composition capable of releasing the second detectable label. In other embodiments, the composition capable of releasing the first detectable label is different from the composition capable of releasing the second detectable label.

In some embodiments, the first detectable label is capable of being detected by a surface acoustic wave device. Optionally, the first detectable label is selected from the group consisting of a magnetic particle, a large metal particle and a spore. In some embodiments, the first detectable label is capable of being detected by a field effect transistor. Optionally, the first detectable label is selected from the group consisting of a magnetic particle, a large metal particle and an ionic solution. In some embodiments, the first detectable label is selected from the group consisting of a fluorescent label, an enzymatic label, a radioactive label, a fluorogenic substrate, and a colorimetric substrate.

In some embodiments, the second detectable label is capable of being detected by a surface acoustic wave device. Optionally, the second detectable label is selected from the group consisting of a magnetic particle, a large metal particle and a spore. In some embodiments, the second detectable label is capable of being detected by a field effect transistor. Optionally, the second detectable label is selected from the group consisting of a magnetic particle, a large metal particle and an ionic solution. In some embodiments, the second detectable label is selected from the group consisting of a fluorescent label, an enzymatic label, a radioactive label, a fluorogenic substrate and a colorimetric substrate.

In some embodiments, the method is performed on a microfluidics device. Optionally, the first antibody conjugate or the first aptamer conjugate is released from a first channel in the microfluidics device. The second antibody conjugate or the second aptamer conjugate may be released from a second channel in the microfluidics device. In some embodiments, the release of the first antibody conjugate or the first aptamer conjugate from the first channel and the first detection step occur before the release of the second antibody conjugate or the second aptamer conjugate from the second channel. Optionally, the first and second antibody conjugates or the first and second aptamer conjugates are released simultaneously and the first and second detection steps take place in different channels of the microfluidics device.

In some embodiments, the method according to the third aspect further comprises:
(1) contacting the sample with one or more additional capture molecules, wherein each of the one or more additional capture molecules is attached to a scaffold or is capable of binding to a scaffold and binds a different marker than the first capture molecule, the second capture molecule and any other additional capture molecule;
(2) contacting the one or more additional markers with a composition comprising one or more additional antibody conjugates, wherein each of the one or more additional antibody conjugates is an antibody conjugate according to the first aspect of the present disclosure, and wherein the one or more additional antibody conjugates recognize different epitopes on the markers than the one or more capture molecules;
(3) contacting the one or more marker-bound additional antibody conjugates with a composition capable of releasing one or more additional detectable labels from the amphiphilic lipid vesicle on the one or more additional antibody conjugates;
(4) performing one or more additional detection steps to detect the one or more additional detectable labels.

In some embodiments, the method further comprises the use of one or more additional aptamer conjugates that binds one or more additional markers and comprises one or more additional detectable labels. Optional the method further comprises the use of at least one additional antibody conjugate and at least one additional aptamer conjugate, wherein the additional antibody conjugate and the additional aptamer conjugate bind different additional markers and comprises additional detectable labels.

In some embodiments, the detection of the marker is possible or improved as a result of signal amplification. The one or more additional capture molecules may comprise capture antibodies. Optionally, the one or more capture molecules comprise capture aptamers. In some embodiments, the one or more capture molecules comprise at least one capture antibody and at least one capture aptamer.

In some embodiments, the first marker is a biomarker, an environmental marker, an allergen, or a microorganism. Optionally, the microorganism is selected from the group consisting of a bacterium, a fungus, an archaeon, an alga, a protozoan and a virus. In some embodiments, the second marker is a biomarker, an environmental marker, an allergen, or a microorganism. Optionally, the microorganism is selected from the group consisting of a bacterium, a fungus, an archaeon, an alga, a protozoan and a virus. The sample may be an environmental sample, a food sample, or a sample obtained from a subject.

A fourth aspect of the present disclosure provides a microfluidics device comprising:
(a) means for receiving a sample;
(b) a capture molecule, wherein the capture molecule is affixed to a scaffold or is capable of binding to the scaffold and binds a marker in the sample;
(c) means for contacting the sample with the capture molecule;
(d) means for contacting the marker with a composition comprising an antibody conjugate, wherein the antibody conjugate is the antibody conjugate according to the first aspect of the disclosure and binds to a different epitope of the marker than the capture molecule;
(e) means for contacting the marker-bound antibody conjugate with a composition capable of releasing a detectable label from the amphiphilic lipid vesicle on the antibody conjugate; and
(f) a detector for the detectable label.

This aspect of the disclosure also provides a microfluidics device comprising an aptamer conjugate, wherein the aptamer conjugate is an aptamer conjugate according to the first aspect of the invention.

In some embodiments, the scaffold is the detector for the detectable label. In other embodiments, the scaffold is adjacent to the detector for the detectable label. In other embodiments, the capture molecule is bound to a magnetic bead or a metallic bead, wherein the capture molecule binds to the scaffold upon the cycling of an electric current or a magnetic field. In other embodiments, the device further comprises means for transporting the detectable label to the detector for the detectable label. Optionally, the capture molecule is a capture antibody. In some embodiments, the capture molecule is a capture aptamer.

In some embodiments, the detector for the detectable label is a surface acoustic wave device. Optionally, the detectable label is selected from the group consisting of a magnetic particle, a large metal particle and a spore. In other embodiments, the detector for the detectable label is a field effect transistor. Optionally, the detectable label is selected from the group consisting of a magnetic particle, a large metal particle, and an ionic solution. In other embodiments, the detector for the detectable label is selected from the group consisting of a fluorescent label detector, an enzymatic label detector, a radioactive label detector, and a colorimetric label detector. Optionally, the detectable label is selected from the group consisting of a fluorescent label, an enzymatic label, a radioactive label, a fluorogenic substrate and a colorimetric substrate.

In some embodiments, the device further comprises means for removing unbound marker. Optionally, the device comprise means for washing the capture molecule-bound marker. In other embodiments, the device further comprises means for removing unbound antibody conjugate or unbound aptamer conjugate. In other embodiments, the device comprises means for washing the marker-bound antibody conjugate or the marker-bound aptamer conjugate.

In some embodiments, the marker is a biomarker, an environmental marker, an allergen or a microorganism. Optionally, the microorganism is selected from the group consisting of a bacterium, a fungus, an archaeon, an alga, a protozoan and a virus. In other embodiments, the sample is an environmental sample, a food sample, or a sample obtained from a subject.

In some embodiments, the device comprises means for cycling an electric field or a magnetic field.

In some embodiments, the composition capable of releasing the detectable label comprises a detergent. Optionally, the detergent is a non-ionic detergent. In some embodiments, the composition capable of releasing the detectable label comprises an enzyme.

A fifth aspect of the present disclosure provides a microfluidics device comprising:
(a) means for receiving a sample;
(b) a first capture molecule, wherein the first capture molecule is affixed to a first scaffold or is capable of binding to the scaffold and binds a first marker in the sample;
(c) means for contacting the sample with the first capture molecule;
(d) a second capture molecule, wherein the second capture molecule is affixed to a second scaffold or is capable of binding to the scaffold and binds a second marker in the sample, wherein the first marker is different from the second marker;
(e) means for contacting the sample with the second capture molecule;
(f) means contacting the first marker with a composition comprising a first antibody conjugate, wherein the first antibody conjugate is an antibody conjugate according to the first aspect of the disclosure and binds to a different epitope of the first marker than the first molecule antibody;
(g) means contacting the second marker with a composition comprising a second antibody conjugate, wherein the second antibody conjugate is an antibody conjugate according to the first aspect of the disclosure and binds to a different epitope of the second marker than the second capture molecule;
(h) means for contacting the first marker-bound first antibody conjugate with a composition capable of releasing a first detectable label from the amphiphilic lipid vesicle on the first antibody conjugate;
(i) means for contacting the second marker-bound second antibody conjugate with a composition capable of releasing a second detectable label from the amphiphilic lipid vesicle on the second antibody conjugate;
(j) a first detector for the first detectable label; and
(k) a second detector for the second detectable label.

This aspect of the disclosure also provides a microfluidics device comprising a first aptamer conjugate that binds a first marker and comprises a first detectable label and a second aptamer conjugate that binds a second marker and comprises a second detectable label, wherein the first and second aptamer conjugates are aptamer conjugates according the first aspect of the disclosure. This aspect of the disclosure further provides a microfluidics device comprising an antibody conjugate that binds a first marker and comprises a first detectable label and an aptamer conjugate that binds a second marker and comprises a second detectable label, wherein the antibody conjugate and the aptamer conjugate are an antibody conjugate and an aptamer conjugate according to the first aspect of the disclosure.

In some embodiments, the first and second detectable labels are the same. Optionally, the first and second detectors are the same device. In some embodiments, the first detectable label is different from the second detectable label. Optionally, the first detector is different from the second detector. In some embodiments, the first and second detectable labels can be detected by the same detector.

In some embodiments, the first and second scaffolds are the same. In other embodiments, the first and second scaffolds are the different. Optionally, the first scaffold is the first detector. The first scaffold may be adjacent to the first detector.

In some embodiments, the first capture molecule is bound to a magnetic bead or a metallic bead, wherein the first capture molecule binds to the first scaffold upon the cycling of an electric current or a magnetic field. In some embodiments, the first capture molecule is a capture antibody. Optionally, the first capture molecule is a capture aptamer.

In some embodiments, the device further comprises means for transporting the first detectable label to the first detector.

In some embodiments, the second scaffold is the second detector. In other embodiments, the second scaffold is adjacent to the second detector. Optionally, the second capture molecule is bound to a magnetic bead or a metallic bead, wherein the second capture molecule binds to the second scaffold upon the cycling of an electric current or a magnetic field. In some embodiments, the second capture molecule is a capture antibody. Optionally, the second capture molecule is a capture aptamer. In some embodiments, the device further comprises means for transporting the second detectable label to the second detector.

In some embodiments, the first antibody conjugate or the first aptamer conjugate is released from a first channel Optionally, the second antibody conjugate or the second aptamer conjugate is released from a second channel. In some embodiments, the first antibody conjugate or the first aptamer conjugate is released from the first channel, and the first detector detects the first detectable label prior to the release of the second antibody conjugate or the second aptamer conjugate from the second channel.

In some embodiments, the first detector is a surface acoustic wave device. Optionally, the first detectable label is selected from the group consisting of a magnetic particle, a large metal particle and a spore. In other embodiments, the first detector is a field effect transistor. Optionally, the first detectable label is selected from the group consisting of a magnetic particle, a large metal particle and an ionic solution. In some embodiments, the first detector is selected from the group consisting of a fluorescent label detector, an enzymatic label detector, a radioactive label detector and a colorimetric label detector. Optionally, the first detectable label is selected from the group consisting of a fluorescent label, an enzymatic label, a radioactive label, a fluorogenic substrate and a colorimetric substrate.

In some embodiments, the second detector is a surface acoustic wave device. Optionally, the second detectable label is selected from the group consisting of a magnetic particle, a large metal particle and a spore. In other embodiments, the second detector is a field effect transistor. Optionally, the second detectable label is selected from the group consisting of a magnetic particle, a large metal particle and an ionic solution. In some embodiments, the second detector is selected from the group consisting of a fluorescent label detector, an enzymatic label detector, a radioactive label detector and a colorimetric label detector. Optionally, the second detectable label is selected from the group consisting of a fluorescent label, an enzymatic label, a radioactive label, a fluorogenic substrate and a colorimetric substrate.

In some embodiments, the microfluidics device further comprises (i) one or more additional capture molecules that bind to one or more additional scaffolds or are capable of binding one or more additional scaffolds and bind one or more additional markers in the sample, wherein the one or more additional markers a different from the first marker, the second marker and any other additional marker; and (ii) one or more additional antibody conjugates or one or more additional aptamer conjugates comprising one or more additional detectable labels, wherein the one or more additional antibody conjugates or the one or more additional aptamer conjugates bind the one or more additional markers at different epitopes than the one or more additional capture molecules. Optionally, the microfluidics device further comprises at least one additional antibody conjugate and at least one additional aptamer conjugate, wherein the additional antibody conjugate and the additional aptamer conjugates bind different markers and comprise an addition detectable label. In some embodiments, the one or more additional capture molecules comprise one or more capture antibodies. Optionally, the one or more additional capture molecules comprise one or more capture aptamers. In some embodiments, the one or more capture molecules comprise at least one capture antibody and at least one capture aptamer.

In some embodiments, the one or more additional detectable labels are the same as the first and second detectable labels. In other embodiments, wherein each of the one or more additional detectable labels is different from the first detectable label, the second detectable label, and any other additional detectable labels.

In some embodiments, the microfluidics device further comprises one or more additional detectors for detecting the one or more additional detectable labels.

In some embodiments, the microfluidics device further comprises means for removing unbound first marker. Optionally, the microfluidics device comprises means for washing the capture molecule-bound first marker. In some embodiments, the microfluidics device further comprises means for removing unbound second marker. Optionally, the microfluidics device comprises means for washing the capture molecule-bound second marker. In some embodiments, the microfluidics device further comprises means for removing unbound first antibody conjugate or unbound first aptamer conjugate. Optionally, the microfluidics device comprises means for washing the marker-bound first antibody conjugate or the marker-bound first aptamer conjugate. In some embodiments, the microfluidics device further comprises means for removing unbound second antibody conjugate or unbound second aptamer conjugate. Optionally, the microfluidics device comprises means for washing the marker-bound second antibody conjugate or the marker-bound second aptamer conjugate. In some embodiments, the device further comprises means for removing unbound one or more additional markers. Optionally, the microfluidics device comprises means for washing the capture molecule-bound one or more additional markers. In some embodiments, the device further comprises means for removing unbound one or more additional antibody conjugates or unbound one or more additional aptamer conjugates. Optionally, the microfluidics device comprises means for washing the marker-bound one or more additional antibody conjugates or the marker-bound one or more additional aptamer conjugates. In some embodiments, the device comprises means for cycling an electric field or a magnetic field.

In some embodiments, the first marker is a biomarker, an environmental marker, an allergen, or a microorganism. Optionally, the microorganism is selected from the group consisting of a bacterium, a fungus, an archaeon, an alga, a protozoan and a virus. In some embodiments, the second marker is a biomarker, an environmental marker, an allergen, or a microorganism. Optionally, the microorganism is selected from the group consisting of a bacterium, a fungus, an archaeon, an alga, a protozoan and a virus. In some embodiments, the one or more additional markers are biomarkers, environmental markers, allergens, or microorganisms. Optionally, the microorganism is selected from the group consisting of a bacterium, a fungus, an archaeon, an alga, a protozoan and a virus. In some embodiments, the sample is an environmental sample, a food sample or a sample obtained from a subject.

In some embodiments, the composition capable of releasing the first detectable label comprises a detergent. Optionally, the detergent is a non-ionic detergent. In some embodiments, the composition capable of releasing the first detectable label comprises an enzyme. In some embodiments, the composition capable of releasing the second detectable label comprises a detergent. Optionally, the detergent is a non-ionic detergent. In some embodiments, the composition capable of releasing the second detectable label comprises an enzyme. In some embodiments, the composition capable of releasing the one or more additional detectable labels comprises a detergent. Optionally, the detergent is a non-ionic detergent. In some embodiments, the composition capable of releasing the one or more additional detectable labels comprises an enzyme.

Particular embodiments of the disclosure are set forth in the following numbered paragraphs:

1. An antibody conjugate comprising an antibody linked to an amphiphilic lipid vesicle, wherein the vesicle comprises a detectable label.

2. The antibody conjugate according to paragraph 1, wherein the amphiphilic lipid vesicle comprises a phospholipid.

3. The antibody according to paragraph 2, wherein the phospholipid is selected from the group consisting of a phosphatidic acid, a phosphatidylethanolamine, a phosphatidylcholine, a phosphatidylserine, a phosphatidylglycerol, a phosphatidylinositol and combinations thereof.

4. The antibody conjugate according to paragraph 3, wherein the phospholipid is phosphatidylcholine.

5. The antibody conjugate according to paragraph 1, wherein the amphiphilic lipid vesicle comprises lecithin.

6. The antibody conjugate according to paragraph 1, wherein the lipid vesicle consists of lecithin.

7. The antibody conjugate according to any one of paragraphs 1-6, wherein the lipid vesicle membrane comprises from 1 to 10 lipid bilayers.

8. The antibody conjugate according to any one of paragraphs 1-7, wherein the lipid vesicle membrane is uniform.

9. The antibody conjugate according to any one of paragraphs 1-8, wherein the lipid vesicle is detergent-soluble.

10. The antibody conjugate according to paragraph 9, wherein the detergent is a non-ionic detergent.

11. The antibody conjugate according to any one of paragraphs 1-8, wherein the lipid vesicle is susceptible to disruption.

12. The antibody conjugate according to paragraph 11, wherein the disruption is enzymatic or by using antimicrobial peptides.

13. The antibody conjugate according to any one of paragraphs 1-12, wherein at least 90% of the detectable label remains in the vesicle for at least one month.

14. The antibody conjugate according to paragraph 13, wherein at least 90% of the detectable label remains in the vesicle for at least three months.

15. The antibody conjugate according to any one of paragraphs 1-14, wherein the detectable label is capable of being detected by a surface acoustic wave device.

16. The antibody conjugate according to paragraph 15, wherein the detectable label is selected from the group consisting of a magnetic particle, a metal particle, a particle of 1 pg or greater, a charged particle and a spore or a combination thereof.

17. The antibody conjugate according to any one of paragraphs 1-14, wherein the detectable label is capable of being detected by a field effect transistor.

18. The antibody conjugate according to paragraph 17, wherein the detectable label is selected from the group consisting of a magnetic particle, a metal particle, a charged particle and an ionic solution or a combination thereof.

19. The antibody conjugate according to paragraph 18, wherein the ionic solution comprises a metal ion.

20. The antibody conjugate according to paragraph 19, wherein the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.

21. The antibody conjugate according to any one of paragraphs 1-14 wherein the detectable label is a fluorescent label, a fluorogenic label, a dye, a colorimetric label, a magnetic label, a radioactive label, a luminescent label, a chemiluminescent label, and an enzymatic label or a combination thereof.

22. The antibody conjugate according to any one of paragraphs 1-21, wherein the antibody is linked to the lipid vesicle by a linker.

23. The antibody conjugate according to paragraph 22, wherein the linker is a peptide linker.

24. The antibody conjugate according to paragraph 23, wherein the peptide linker is between 5 and 50 amino acids long.

25. The antibody conjugate according to paragraph 23 or 24, wherein the peptide linker comprises a protease cleavage site.

26. The antibody conjugate according to paragraph 22, wherein the linker is released by cleavage of a disulfide bond.

27. A method for detecting a marker in a sample, the method comprising:

(a) contacting the sample with a capture molecule that binds the marker, wherein the capture molecule is affixed to a scaffold or is capable of being affixed to a scaffold,

(b) contacting the marker with a composition comprising the antibody conjugate according to any one of paragraphs 1-26, wherein the antibody conjugate binds to a different epitope on the marker than the capture molecule;

(c) contacting the marker-bound antibody conjugate with conditions capable of releasing the detectable label from the amphiphilic lipid vesicle on the antibody conjugate; and

(d) detecting the detectable label.

28. The method according to paragraph 25, wherein the conditions capable of releasing the detectable label from the amphiphilic lipid vesicle on the antibody conjugate comprise contacting the vesicle with a composition.

29. The method according to paragraph 25, wherein the composition is selected from the group consisting of a low-pH composition, a composition comprising a detergent, a composition comprising an enzyme and a composition comprising a non-detergent chemical compound.

30. The method according to paragraph 29, wherein the detergent is a non-ionic detergent.

31. The method according to any one of paragraphs 27-30, wherein the scaffold is a detector for the detectable label.

32. The method according to any one of paragraphs 27-30, wherein the scaffold is adjacent to a detector for the detectable label.

33. The method according to any one of paragraphs 27-32, wherein the capture molecule is bound to a magnetic bead or a metallic bead, wherein the capture molecule binds to the scaffold upon the cycling of an electric current or a magnetic field.

34. The method according to any one of paragraphs 27-30, wherein the detection step comprises the step of transporting the detectable label to a detector for the detectable label.

35. The method according to any one of paragraphs 27-34, wherein the method further comprises one or more steps of washing the marker-bound antibody conjugate before the releasing step.

36. The method according to any one of paragraphs 27-35, wherein the method further comprises one or more steps of washing the capture molecule-bound marker.

37. The method according to any one of paragraphs 27-36, wherein the detectable label is capable of being detected by a surface acoustic wave device.

38. The method according to paragraph 37, wherein the detectable label is selected from the group consisting of a magnetic particle, a metal particle, a particle of 1 pg or greater, and a spore or a combination thereof.

39. The method according to any one of paragraphs 27-36, wherein the detectable label is capable of being detected by a field effect transistor (FET).

40. The method according to paragraph 39, wherein the detectable label is selected from the group consisting of a magnetic particle, a metal particle, a charged particle and an ionic solution or a combination thereof.

41. The method according to paragraph 40, wherein the ionic solution comprises a metal ion.

42. The method according to paragraph 41, wherein the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.

43. The method according to paragraph 41 or 42, further comprising contacting the metal ion with a metal ion chelator or metal ion derivatized chelator, wherein the metal ion chelator or metal ion derivatized chelator is located at or near a detector for the detectable label.

44. The method according to paragraph 43, wherein the metal ion is Ca²⁺.

45. The method according to paragraph 44, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium salt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.

46. The method according to any one of paragraphs 27-36, wherein the detectable label is selected from the group consisting of a fluorescent label, a fluorogenic label, a dye, a colorimetric label, a magnetic label, a radioactive label, a luminescent label, a chemiluminescent label, and an enzymatic label or a combination thereof.

47. The method according to any one of paragraphs 27-46, wherein the method is performed on a microfluidics device.

48. The method according to any one of paragraphs 27-47, wherein the detection of the marker is possible or improved as a result of signal amplification.

49. The method according to any one of paragraphs 27-48, wherein the marker is a biomarker, an environmental marker, an allergen, or a microorganism.

50. The method according to paragraph 49, wherein the microorganism is selected from the group consisting of a bacterium, a fungus, an archaeon, an alga, a protozoan and a virus.

51. The method according to any one of paragraphs 27-50, wherein the sample is an environmental sample, a food sample, or a sample obtained from a subject.

52. A method of detecting one of a plurality of markers in a sample, the method comprising:

(a) contacting the sample with a first capture molecule and a second capture molecule, wherein the first capture molecule is affixed to a first scaffold or is capable of being affixed to the first scaffold and binds a first marker, wherein the second capture molecule is affixed to a second scaffold or is capable of being affixed to the second scaffold and binds a second marker, wherein the first marker is different from the second marker;

(b) contacting the first marker with a composition comprising a first antibody conjugate, wherein the first antibody conjugate is an antibody conjugate according to any one of paragraphs 1-26, and wherein the first antibody conjugate recognizes a different epitope on the first marker than the first capture molecule;

(c) contacting the second marker with a composition comprising a second antibody conjugate, wherein the second antibody conjugate is an antibody conjugate according to any one of paragraphs 1-26, and wherein the second antibody conjugate recognizes a different epitope on the second marker than the second capture molecule;

(d) contacting the first marker-bound first antibody conjugate with conditions capable of releasing a first detectable label from the amphiphilic lipid vesicle on the first antibody conjugate;

(e) contacting the second marker-bound second antibody conjugate with conditions capable of releasing a second detectable label from the amphiphilic lipid vesicle on the second antibody conjugate;

(f) performing a first detection step to detect the first detectable label; and

(g) performing a second detection step to detect the second detectable label.

53. The method according to paragraph 52, wherein the first scaffold is a detector for the first detectable label.

54. The method according to paragraph 52, wherein the first scaffold is adjacent to a detector for the first detectable label.

55. The method according to any one of paragraphs 52-54, wherein the first capture molecule is bound to a magnetic bead or a metallic bead, wherein the first capture molecule binds to the first scaffold upon the cycling of an electric current or a magnetic field.

56. The method according to any one of paragraphs 52-55, wherein the second scaffold is a detector for the second detectable label.

57. The method according to any one of paragraphs 52-55, wherein the second scaffold is adjacent to a detector for the second detectable label.

58. The method according to any one of paragraphs 52-57, wherein the second capture molecule is bound to a magnetic bead or a metallic bead, wherein the second capture molecule binds to the second scaffold upon the cycling of an electric current.

59. The method according to any one of paragraphs 52-58, wherein the first and second scaffolds are the same.

60. The method according to any one of paragraph 52-58, wherein the first and second scaffolds are different.

61. The method according to any one of paragraphs 52 and 56-60, wherein the first detection step comprises the step of transporting the first detectable label to a detector for the first detectable label.

62. The method according to any one of paragraphs 52-55 and 58-60, wherein the second detection step comprises the step of transporting the second detectable label to a detector for the second detectable label.

63. The method according to any one of paragraphs 52-62, wherein the method further comprises the step of washing the capture molecule-bound markers.

64. The method according to any one of paragraphs 52-63, wherein the first marker is contacted with the first capture molecule before the first marker is contacted with the first antibody conjugate; and wherein the method further comprises the step of washing the marker-bound first antibody conjugate before releasing the first detectable label.

65. The method according to any one of paragraphs 52-64, wherein the method further comprises the step of washing the marker-bound second antibody conjugate before releasing the second detectable label.

66. The method according to any one of paragraphs 52-65, wherein the first detectable label is different from the second detectable label, and the marker is detected by which detectable label is present.

67. The method according to any one of paragraphs 52-65, wherein the first detectable label and the second detectable label are the same, and the marker is detected by whether the detectable label is present in the first detection step or the second detection step.

68. The method according to any one of paragraphs 52-67, further comprising the step of washing the capture molecule-bound first marker.

69. The method according to any one of paragraphs 52-68, further comprising the step of washing the capture molecule-bound second marker.

70. The method according to any one of paragraphs 52-68, further comprising the step of washing the detector prior to the first detection step.

71. The method according to any one of paragraphs 52-70, further comprising the step of washing the detector prior to the second detection step.

72. The method according to any one of paragraphs 52-71, wherein the conditions capable of releasing the first detectable label from the amphiphilic lipid vesicle on the first antibody conjugate comprise contacting the vesicle on the first antibody conjugate with a composition capable of releasing the first detectable label.

73. The method according to paragraph 72, wherein the composition capable of releasing the first detectable label comprises a detergent.

74. The method according to paragraph 73, wherein the detergent is a non-ionic detergent.

75. The method according to paragraph 72, wherein the composition capable of releasing the first detectable label comprises an enzyme.

76. The method according to any one of paragraphs 52-75, wherein the conditions capable of releasing the second detectable label from the amphiphilic lipid vesicle on the second antibody conjugate comprise contacting the vesicle on the second antibody conjugate with a composition capable of releasing the first detectable label.

77. The method according to paragraph 76, wherein the composition capable of releasing the second detectable label comprises a detergent.

78. The method according to paragraph 77, wherein the detergent is a non-ionic detergent.

79. The method according to paragraph 76, wherein the composition capable of releasing the second detectable label comprises an enzyme.

80. The method according to any one of paragraphs 72-79, wherein the composition capable of releasing the first detectable label is the same as the composition capable of releasing the second detectable label.

81. The method according to any one of paragraphs 72-79, wherein the composition capable of releasing the first detectable label is different from the composition capable of releasing the second detectable label.

82. The method according to any one of paragraphs 52-81, wherein the first detectable label is capable of being detected by a surface acoustic wave device.

83. The method according to paragraph 82, wherein the first detectable label is selected from the group consisting of a magnetic particle, a metal particle, a particle of 1 pg or greater, a charged particle and a spore or a combination thereof.

84. The method according to any one of paragraphs 52-81, wherein the first detectable label is capable of being detected by a field effect transistor (FET).

85. The method according to paragraph 84, wherein the first detectable label is selected from the group consisting of a magnetic particle, a metal particle, a charged particle and an ionic solution or a combination thereof. 86. The method according to paragraph 85, wherein the ionic solution comprises a metal ion.

87. The method according to paragraph 86, wherein the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.

88. The method according to paragraph 86 or 87, further comprising contacting the metal ion with a metal ion chelator or metal ion derivatized chelator, wherein the metal ion chelator or metal ion derivatized chelator is located at or near the detector for the first detectable marker.

89. The method according to paragraph 88, wherein the metal ion is Ca²⁺.

90. The method according to paragraph 89, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium salt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.

91. The method according to any one of paragraphs 52-81, wherein the first detectable label is selected from the group consisting of a fluorescent label, fluorogenic labels, dyes, colorimetric labels, radioactive labels, luminescent labels, chemiluminescent labels, and enzymatic label or a combination thereof.

92. The method according to any one of paragraphs 52-91, wherein the second detectable label is capable of being detected by a surface acoustic wave device.

93. The method according to paragraph 92, wherein the second detectable label is selected from the group consisting of a magnetic particle, a metal particle, a particle of 1 pg or greater, a charged particle and a spore or a combination thereof.

94. The method according to any one of paragraphs 52-91, wherein the second detectable label is capable of being detected by a field effect transistor.

95. The method according to paragraph 94, wherein the second detectable label is selected from the group consisting of a magnetic particle, a metal particle, a charged particle and an ionic solution or a combination thereof 96. The method according to paragraph 95, wherein the ionic solution comprises a metal ion.

97. The method according to paragraph 96, wherein the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.

98. The method according to paragraph 96 or 97, further comprising contacting the metal ion with a metal ion chelator or metal ion derivatized chelator, wherein the metal ion chelator or metal ion derivatized chelator is located at or near the detector for the second detectable label.

99. The method according to paragraph 98, wherein the metal ion is Ca²⁺.

100. The method according to paragraph 99, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium salt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.

101. The method according to any one of paragraphs 52-91, wherein the second detectable label is selected from the group consisting of a fluorescent label, a fluorogenic label, a dye, a colorimetric label, a magnetic label, a radioactive label, a luminescent label, a chemiluminescent label, and an enzymatic label or a combination thereof.

102. The method according to any one of paragraphs 52-101, wherein the method is performed on a microfluidics device.

103. The method according to paragraph 102, wherein the first antibody conjugate is released from a first channel in the microfluidics device.

104. The method according to paragraph 102 or 103, wherein the second antibody conjugate is released from a second channel in the microfluidics device.

105. The method according to paragraph 104, wherein the release of the first antibody conjugate from the first channel and the first detection step occur before the release of the antibody conjugate from the second channel.

106. The method according to any one of paragraphs 52-105, wherein the method further comprises:

(1) contacting the sample with one or more additional capture molecules, wherein each of the one or more additional capture molecules is attached to a scaffold or is capable of binding to a scaffold and binds a different marker than the first capture molecule, the second capture molecule and any other additional capture molecule;

(2) contacting the one or more additional markers with a composition comprising one or more additional antibody conjugates, wherein each of the one or more additional antibody conjugates is an antibody conjugate according to any one of paragraphs 1-26, and wherein the one or more additional antibody conjugates recognize different epitopes on the different markers than the one or more capture molecules;

(3) contacting the one or more marker-bound additional antibody conjugates with a composition capable of releasing one or more additional detectable labels from the amphiphilic lipid vesicle on the one or more additional antibody conjugates;

(4) performing one or more additional detection steps to detect the one or more additional detectable labels.

107. The method according to any one of paragraphs 52-106, wherein the detection of the marker is possible or improved as a result of signal amplification.

108. The method according to any one of paragraphs 52-107, wherein the first marker is a biomarker, an environmental marker, an allergen, or a microorganism.

109. The method according to paragraph 108, wherein the microorganism is selected from the group consisting of a bacterium, a fungus, an archaeon, an alga, a protozoan and a virus.

110. The method according to any one of paragraphs 52-108, wherein the second marker is a biomarker, an environmental marker, an allergen, or a microorganism.

111. The method according to paragraph 110, wherein the microorganism is selected from the group consisting of a bacterium, a fungus, an archaeon, an alga, a protozoan and a virus.

112. The method according to any one of paragraphs 52-111, wherein the sample is an environmental sample, a food sample, or a sample obtained from a subject.

113. A microfluidics device comprising:

(a) means for receiving a sample;

(b) a capture molecule, wherein the capture molecule is affixed to a scaffold or is capable of binding to the scaffold and binds a marker in the sample;

(c) means for contacting the sample with the capture molecule;

(d) means for contacting the marker with a composition comprising an antibody conjugate, wherein the antibody conjugate is an antibody conjugate according to any one of paragraphs 1-26 and binds to a different epitope of the marker than the capture molecule;

(e) means for contacting the marker-bound antibody conjugate with conditions capable of releasing a detectable label from the amphiphilic lipid vesicle on the antibody conjugate; and

(f) a detector for the detectable label.

114. The microfluidics device according to paragraph 113, wherein the scaffold is a detector for the detectable label.

115. The microfluidics device according to paragraph 113, wherein the scaffold is adjacent to a detector for the detectable label.

116. The microfluidics device according to any one of paragraphs 113-116, wherein the capture molecule is bound to a magnetic bead or a metallic bead, wherein the capture molecule binds to the scaffold upon the cycling of an electric current.

117. The microfluidic device according to any one of paragraphs 113-116, wherein the device further comprises means for transporting the detectable label to the detector for the detectable label.

118. The microfluidics device according to any one of paragraphs 113-117, wherein the detector for the detectable label is a surface acoustic wave device.

119. The microfluidics device according to paragraph 118, wherein the detectable label is selected from the group consisting of a magnetic particle, a metal particle, a particle of 1 pg or greater, and a spore or a combination thereof 120. The microfluidics device according to any one of paragraphs 113-118, wherein the detector for the detectable label is a field effect transistor (FET).

121. The microfluidics device according to paragraph 119 or 120, wherein the detectable label is selected from the group consisting of a magnetic particle, a metal particle, a charged particle and an ionic solution or a combination thereof 122. The microfluidics device according to paragraph 121, wherein the ionic solution comprises a metal ion.

123. The microfluidics device according to paragraph 122, wherein the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.

124. The microfluidics device according to paragraphs 122 or 123, further comprising contacting the metal ion with a metal ion chelator or metal ion derivatized chelator, wherein the metal ion chelator or metal ion derivatized chelator is located at or near the detector.

125. The microfluidics device according to paragraph 124, wherein the metal ion is Ca²⁺.

126. The microfluidics device according to paragraph 125, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium salt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.

127. The microfluidics device according to any one of paragraphs 113-117, wherein the detector for the detectable label is selected from the group consisting of a fluorescent label, a fluorogenic label, a dye, a colorimetric label, a magnetic label, a radioactive label, a luminescent label, a chemiluminescent label, and an enzymatic label or a combination thereof.

128. The microfluidics device according to any one of paragraphs 113-127, wherein the device further comprises means for washing the capture molecule-bound marker.

129. The microfluidics device according to any one of paragraphs 113-128, wherein the device further comprises means for washing the marker-bound antibody conjugate.

130. The microfluidics device according to any one of paragraphs 113-129, wherein the marker is a biomarker, an environmental marker, an allergen, or a microorganism.

131. The microfluidics device according to paragraph 130, wherein the microorganism is selected from the group consisting of a bacterium, a fungus, an archaeon, an alga, a protozoan and a virus.

132. The microfluidics device according to any one of paragraphs 113-131, wherein the sample is an environmental sample, a food sample, or a sample obtained from a subject.

133. The microfluidics device according to any one of paragraphs 113-132, wherein the device comprises means for cycling an electric field or a magnetic field.

134. The microfluidics device according to any one of paragraphs 113-133, wherein the conditions capable of releasing the detectable label comprises a composition comprising a detergent.

135. The microfluidics device according to paragraph 134, wherein the detergent is a non-ionic detergent.

136. The microfluidics device according to any one of paragraphs 113-133, wherein the conditions capable of releasing the detectable label comprises a composition comprising an enzyme.

137. A microfluidics device comprising
(a) means for receiving a sample;

(b) a first capture molecule, wherein the first capture molecule is affixed to a first scaffold or is capable of binding to the scaffold and binds a first marker in the sample;

(c) means for contacting the sample with the first capture molecule;

(d) a second capture molecule, wherein the second capture molecule is affixed to a second scaffold or is capable of binding to the scaffold and binds a second marker in the sample, wherein the first marker is different from the second marker;

(e) means for contacting the sample with the second capture molecule;

(f) means for contacting the first marker with a composition comprising a first antibody conjugate, wherein the first antibody conjugate is an antibody conjugate according to any one of paragraphs 1-26 and binds to a different epitope of the first marker than the first capture molecule;

(g) means for contacting the second marker with a composition comprising a second antibody conjugate, wherein the second antibody conjugate is an antibody conjugate according to any one of paragraphs 1-26 and binds to a different epitope of the second marker than the second capture molecule;

(h) means for contacting the first marker-bound first antibody conjugate with a composition capable of releasing a first detectable label from the amphiphilic lipid vesicle on the first antibody conjugate;

(i) means for contacting the second marker-bound second antibody conjugate with a composition capable of releasing a second detectable label from the amphiphilic lipid vesicle on the second antibody conjugate;

(j) a first detector for the first detectable label; and

(k) a second detector the second detectable label.

138. The microfluidics device according to paragraph 137, wherein the first and second detectable labels are the same.

139. The microfluidics device according to paragraph 137 or 138, wherein the first and second detectors are the same device.

140. The microfluidics device according to paragraph 137, wherein the first detectable label is different from the second detectable label.

141. The microfluidics device according to paragraph 140, wherein the first and second detectable labels can be detected by the same detector.

142. The microfluidics device according to any one of paragraphs 137-141, wherein the first and second scaffolds are the same.

143. The microfluidics device according to any one of paragraphs 137-141, wherein the first and second scaffolds are different.

144. The microfluidics device according to any one of paragraphs 137-143, wherein the first scaffold is the first detector.

145. The microfluidics device according to any one of paragraphs 137-143, wherein the first scaffold is adjacent to the first detector.

146. The microfluidics device according to any one of paragraphs 137-145, wherein the first capture molecule is bound to a magnetic bead or a metallic bead, wherein the first capture molecule binds to the first scaffold upon the cycling of an electric current.

147. The microfluidics device according to any one of paragraphs 137-146, further comprising means for transporting the first detectable label to the first detector.

148. The microfluidics device according to any one of paragraphs 137-147, wherein the second scaffold is the second detector.

149. The microfluidics device according to any one of paragraphs 137-147, wherein the second scaffold is adjacent to the second detector.

150. The microfluidics device according to any one of paragraphs 137-149, wherein the second capture molecule is bound to a magnetic bead or a metallic bead, wherein the second capture molecule binds to the second scaffold upon the cycling of an electric current.

151. The microfluidics device according to any one of paragraphs 137-150, further comprising means for transporting the second detectable label to the second detector.

152. The microfluidics device according to any one of paragraphs 137-151, wherein the first antibody conjugate is released from a first channel and the second antibody conjugate is released from a second channel.

153. The microfluidics device according to paragraph 152, wherein the first antibody conjugate is released from the first channel and the first detector detects the first detectable label prior to the release of the second antibody conjugate from the second channel.

154. The microfluidics device according to any one of paragraphs 137-153, wherein the first detector is a surface acoustic wave device.

155. The microfluidics device according to paragraph 154, wherein the first detectable label is selected from the group consisting of a magnetic particle, a metal particle, a particle of 1 pg or greater, a charged particle and a spore or a combination thereof.

156. The microfluidics device according to any one of paragraphs 137-153, wherein the first detector is a field effect transistor.

157. The microfluidics device according to paragraph 156, wherein the first detectable label is selected from the group consisting of a magnetic particle, a metal particle, a charged particle and an ionic solution or a combination thereof.

158. The microfluidics device according to any one of paragraphs 137-153, wherein the first detector is selected from the group consisting of a fluorescent label, a fluorogenic label, a dye, a colorimetric label, a magnetic label, a radioactive label, a luminescent label, a chemiluminescent label, and an enzymatic label or a combination thereof.

159. The microfluidics device according to any one of paragraphs 137-158, wherein the second detector is a surface acoustic wave device.

160. The microfluidics device according to paragraph 159, wherein the second detectable label is selected from the group consisting of a magnetic particle, a metal particle, a particle of 1 pg or greater, a charged particle and a spore or a combination thereof.

161. The microfluidics device according to any one of paragraphs 137-158, wherein the second detector is a field effect transistor.

162. The microfluidics device according to paragraph 161, wherein the second detectable label is selected from the group consisting of a magnetic particle, a metal particle, a charged particle and an ionic solution or a combination thereof.

163. The microfluidics device according to paragraph 157 or 162, wherein the ionic solution comprises a metal ion.

164. The microfluidics device according to paragraph 163, wherein the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.

165. The microfluidics device according to paragraph 163 or 164, further comprising contacting the metal ion with a metal ion chelator or metal ion derivatized chelator, wherein the metal ion chelator or metal ion derivatized chelator is located at or near the detector.

166. The microfluidics device according to paragraph 165, wherein the metal ion is Ca²⁺.

167. The microfluidics device according to paragraph 166, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium salt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.

168. The microfluidics device according to any one of paragraphs 137-158, wherein the second detector is selected from the group consisting of fluorescent label, a fluorogenic label, a dye, a colorimetric label, a magnetic label, a radioactive label, a luminescent label, a chemiluminescent label, and an enzymatic label or a combination thereof.

169. The microfluidics device according to any one of paragraphs 137-168, wherein the microfluidics device further comprises one or more additional capture molecules that bind to one or more additional scaffolds or are capable of binding one or more additional scaffolds and bind one or more additional markers in the sample, wherein the one or more additional markers a different from the first marker, the second marker and any other additional marker; and one or more additional antibody conjugates comprising one or more additional detectable labels, wherein the one or more additional antibody conjugates bind the one or more additional markers at different epitopes than the one or more capture molecules.

170. The microfluidics device according to paragraph 169, wherein the one or more additional detectable labels are the same as the first and second detectable labels.

171. The microfluidics device according to paragraph 169, wherein each of the one or more additional detectable labels is different from the first detectable label, the second detectable label, and any other additional detectable labels.

172. The microfluidics device according to 171, wherein the microfluidics device further comprises one or more detectors for detecting the one or more additional detectable labels.

173. The microfluidics device of any one of paragraphs 137-172, wherein the microfluidics device further comprises means for washing the capture molecule-bound first marker.

174. The microfluidics device of any one of paragraphs 137-173, wherein the microfluidics device further comprises means for washing the capture molecule-bound second marker.

175. The microfluidics device of any one of paragraphs 137-174, wherein the microfluidics device further comprises means for washing the marker-bound first antibody conjugate.

176. The microfluidics device of any one of paragraphs 137-174, wherein the microfluidics device further comprises means for washing the marker-bound second antibody conjugate.

177. The microfluidics device according to any one of paragraphs 169-176, wherein the device further comprises means for washing the capture molecule-bound one or more additional markers.

178. The microfluidics device according to any one of paragraphs 169-177, wherein the device further comprises means for washing the marker-bound one or more additional antibody conjugates.

179. The microfluidics device according to any one of paragraphs 137-178, wherein the device comprises means for cycling an electric field or a magnetic field.

180. The microfluidics device according to any one of paragraphs 137-179, wherein the first marker is a biomarker, an environmental marker, an allergen, or a microorganism.

181. The microfluidics device according to paragraph 180, wherein the microorganism is selected from the group consisting of a bacterium, a fungus, an archaeon, an alga, a protozoan and a virus.

182. The microfluidics device according to any one of paragraphs 137-181, wherein the second marker is a biomarker, an environmental marker, an allergen, or a microorganism.

183. The microfluidics device according to paragraph 182, wherein the microorganism is selected from the group consisting of a bacterium, a fungus, an archaeon, an alga, a protozoan and a virus.

184. The microfluidics device according to any one of paragraphs 169-183, wherein the one or more additional markers are biomarkers, environmental markers, allergens, or microorganisms.

185. The microfluidics device according to paragraph 184, wherein the microorganisms are selected from the group consisting of bacteria, fungi, an archaeon, algae, protozoans and viruses.

186. The microfluidics device according to any one of paragraphs 137-185, wherein the sample is an environmental sample, a food sample, or a sample obtained from a subject.

187. The microfluidics device according to any one of paragraphs 137-186, wherein the composition capable of releasing the first detectable label comprises a detergent.

188. The microfluidics device according to paragraph 187, wherein the detergent is a non-ionic detergent.

189. The microfluidics device according to any one of paragraphs 137-186, wherein the composition capable of releasing the first detectable label comprises an enzyme.

190. The microfluidics device according to any one of paragraphs 137-189, wherein the composition capable of releasing the second detectable label comprises a detergent.

191. The microfluidics device according to paragraph 190, wherein the detergent is a non-ionic detergent.

192. The microfluidics device according to any one of paragraphs 137-189, wherein the composition capable of releasing the second detectable label comprises an enzyme.

### BRIEF DESCRIPTION OF DRAWINGS

FIG. 1 shows a schematic side cross sectional representation of a transistor device and liposome immunoassay in accordance with embodiments of the present disclosure;

FIGS. 2A-2D show side cross sectional representations of a scheme for detection of a target analyte in solution using FETs in accordance with embodiments of the present disclosure;

FIG. 3 shows the electrical double-layer length known as the Debye limit for materials' ability to interact with a substrate interface to make a detectable change in the device voltage, in accordance with embodiments of the present disclosure.

FIGS. 4A and 4B are graphs showing I_{d} (drain current) as a function of V_{d} (drain voltage) of measured dry I_{d}-V_{d} curves for the FET transistor that confirms linear drain current dependence for the gate bias based on differing input voltage (−5V, −2V, 0V, 1V, 2V, 3V and 4V), in accordance with embodiments of the present disclosure, with FIG. 4B being an enlargement of the I_{d}-V_{d} curve of FIG. 4A between 3 and 3.5 mA for the different gate voltages.

FIGS. 5A-5E are diagrammatic depictions of a portion of a fluidic circuit used for detection of a target analyte in solution in accordance with embodiments of the present disclosure.

FIG. 6 is a simplified schematic of a circuit for measuring the ion or cation concentration in the buffer with a pair of electrodes after the disruption of the lipid vesicles in the test chamber.

### DETAILED DESCRIPTION OF THE INVENTION

The ability to detect very rare cells or markers at low concentrations in a sample, such as blood or plasma, with accuracy and sensitivity is still a significant problem for molecular diagnostics. Typical protein detection methods, such as ELISAs, are generally not sensitive enough to detect low concentrations of important biological markers. There is a need for cost-effective and rapid analysis methods and devices for the detection of markers, including biomarkers and environmental markers, especially more sensitive, specific, and robust methods and devices.

The present disclosure provides antibody and aptamer conjugates and sensitive, specific, and robust methods and microfluidics devices using such antibody and aptamer conjugates, which can be used to specifically detect markers that are present at a low concentration in the sample, near or below the limit of detection (LOD), such that amplification is necessary.

The LOD of a detection method is the lowest amount of analyte or marker in a sample which can be detected. Accordingly, an improved LOD when the amount of the analyte or marker in the sample which can be detected is decreased or the LOD is reduced. Several approaches for determining the detection limit are possible. For instance, in the detection of bacteria, every test method will have an upper and lower LOD. This is determined by the statistical accuracy with which the analysts are able to count the colonies growing on the plates.

The antibody and aptamer conjugates, methods, and microfluidics devices of the present disclosure permit the detection of markers at very low concentration, which may be due to (a) the immobilization of the marker near the detector and/or by (b) amplifying the signal. The immobilization of the marker may be accomplished by binding a capture molecule, e.g. a capture antibody, to a scaffold. The capture molecule binds the marker. The amplification step may involve binding the immobilized marker with a detection molecule, such as an antibody conjugate of the disclosure, which comprises an amplifiable signal (e.g., a liposome comprising an ion, e.g. a metal ion). After the detection molecule, e.g. an antibody conjugate of the disclosure, is bound to the immobilized marker, the method can optionally comprise a washing step or the microfluidics device can include washing means to remove any unbound detection molecule. In some embodiments, the amplifiable signal is a liposome comprising an ion, e.g. a metal ion, and the signal is amplified by releasing the ions from the liposomes. In some embodiments, the ions are released from the liposomes by contacting the liposomes with a detergent. The signal may be further amplified by attracting the ions toward a detector, e.g. in a microfluidics device.

### 1. General Techniques

Unless otherwise defined herein, scientific and technical terms used in this application shall have the meanings that are commonly understood by those of ordinary skill in the art. Generally, nomenclature used in connection with, and the laboratory procedures techniques performed in pharmacology, cell and tissue culture, analytical chemistry, biochemistry, synthetic organic chemistry, medicinal and pharmaceutical chemistry, molecular biology, cell and cancer biology, neurobiology, neurochemistry, virology, immunology, microbiology, genetics and protein and nucleic acid chemistry, described herein, are those well-known and commonly used in the art. Standard techniques are used for chemical syntheses and chemical analyses. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999).

Enzymatic reactions and purification techniques are performed according to manufacturer's specifications, as commonly accomplished in the art or as described herein.

Throughout this specification and embodiments, the word “comprise,” or variations such as “comprises” or “comprising,” will be understood to imply the inclusion of a stated integer or group of integers, but not the exclusion of any other integer or group of integers. The term “including” is used to mean “including but not limited to.” “Including” and “including but not limited to” are used interchangeably.

It is understood that wherever embodiments are described herein with the language “comprising,” otherwise analogous embodiments described in terms of “consisting of” and/or “consisting essentially of” are also provided.

Any example(s) following the term “e.g.” or “for example” is not meant to be exhaustive or limiting.

Unless otherwise required by context, singular terms shall include pluralities and plural terms shall include the singular.

The articles “a,” “an” and “the” are used herein to refer to one or to more than one (i.e., to at least one) of the grammatical object of the article. By way of example, “an element” means one element or more than one element. As used herein, the term “about” modifying the quantity of an ingredient, parameter, calculation, or measurement in the compositions of the disclosure or employed in the methods of the disclosure refers to a variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within an order of magnitude, typically within 10% of a given value or range, more typically still within 5% of a given value or range. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the invention are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the embodiments of the invention.

Exemplary methods and materials are described herein, although methods and materials similar or equivalent to those described herein can also be used in the practice or testing of the present disclosure. The materials, methods, and examples are illustrative only and not intended to be limiting.

### 2. Definitions

The “affinity binding” of the capture molecule to the marker present in the sample or of the marker and the antibody conjugate of the invention, as referred in the present application, is measured by the dissociation constant or K_{D}. As used herein, the term “affinity binding” in the context of the binding of an antibody to a predetermined antigen is typically a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less. K_{D} values are measured by techniques known by the skilled in the art, such as, for example ELISA, surface plasmon resonance (SPR), fluorescence anisotropy, Bio-Layer Interferometry, typically using OCTET® technology (Octet QKe system, ForteBio) or a KinExA® (Kinetic Exclusion Assay) assay.

The term “antibody,” as used herein, refers to a gamma-globulin, or a fragment thereof, that exhibits a specific binding activity for a target molecule, namely. The term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. Such antibodies may be produced in a variety of ways, including hybridoma cultures, recombinant expression in bacteria or mammalian cell cultures, and recombinant expression in transgenic animals. Also, antibodies can be produced by selecting a sequence from a library of sequences expressed in display systems such as filamentous phage, bacterial, yeast or ribosome. There is abundant guidance in the literature for selecting a particular production methodology, e.g., Chadd and Chamow, Curr. Opin. Biotechnol., 12:188-194 (2001). The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species.

The term “antibody,” as used herein, also includes the term “antigen binding fragment,” which refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)2, and Fv fragments. Antibody fragments of small size, such as Fab and Fv fragments, having no effector functions and limited pharmokinetic activity, may be generated in a bacterial expression system. Single chain Fv fragments show low immunogenicity and are cleared rapidly from the blood.

The term “aptamer,” as used herein, refers to an oligonucleotide (such as, for example, DNA or RNA) or a peptide molecule that binds to a specific target molecule. Aptamers may show a high affinity and specificity for their target molecules. Aptamers may be synthesized by chemical or enzymatic procedures, or a combination thereof. Non-limiting examples of aptamer targets include proteins, peptides, carbohydrates, and small molecules. Aptamer binding is typically determined by its tertiary structure, not its primary sequence. Target recognition and binding are usually determined by three-dimensional, shape-dependent interactions as well as hydrophobic interactions, base-stacking, and intercalation. Methods for generating aptamers that specifically bind a target molecule are known in the art. For example, the skilled artisan may generate aptamers by systematic evolution of ligands by exponential enrichment (SELEX). See, e.g., Darmostuk et al. Biotechnology Advances, 2015, vol. 33(6): 1141-1161. In some embodiments, the aptamer is an oligonucleotide. Optionally, the aptamer comprises DNA residues. In some embodiments, the aptamer comprises RNA residues. Optionally, the oligonucleotide is single-stranded.

As used herein, a “capture molecule” is a molecule used to bind an antigen or marker being assayed via an affinity binding between the capture molecule and the antigen or marker in a liquid phase and affix the captured antigen or marker to a solid phase. The capture molecule may comprise an antibody, a recombinant antibody, a protein, a recombinant protein, small or big organic molecules, or peptide or nucleic acid aptamers. If the capture molecule is an antibody, then it is named as “capture antibody.” If the capture molecule is an aptamer, then it is named as “capture aptamer.” The capture molecule may be affixed to the solid phase. In some embodiments, the capture molecule is capable of being affixed to the solid phase, e.g., upon cycling of a magnetic field or an electric current.

The terms “chelator” and “chelating agent” are used interchangeably herein and refer to a molecule that binds to metal ions and form a complex. The affinity of the chelating agent for the metal ion is measured by the dissociation constant or KID. As used herein, a high-affinity chelator is typically a binding with an affinity corresponding to a K_{D} of about 10⁻⁸ M or less, about 10⁻⁹ M or less, about 10⁻¹⁰ M or less, or about 10⁻¹¹ M or less. K_{D} values are measured by techniques known by the skilled in the art, such as, the pH metric method developed by Moisescu and Pusch (Moisescu, D. G. and Pusch, H. (1975) Pfluegers Arch. 355, 243) or a modified version of said pH metric method (Smith and Miller. (1985). Biochimica et Biophysica Acta; Vol. 839, Issue 3, 287-299).

The terms “derivatized chelator” and “derivatized chelating agent” are used interchangeably herein and refer to chelators that have been chemically altered to permit them to be interposed onto a scaffold or a detector, e.g. a field effect transistor or a component thereof, including a substrate, a carbon nanotube, a dielectric material, a gate, or between a source and a drain. Methods for derivatizing chelators for such disposition are known in the art. For example, pyrenes are known to adsorb to carbon nanotube surfaces through π-π interactions. Additionally, azide chemistry has been demonstrated to be a powerful means to covalently modify carbon nanotubes.

As used herein, the term “calcium chelator” is used to refer to molecules that are able to bind calcium in a selective way because they have higher affinity for calcium than for any other metal ions. Binding to calcium is typically performed through carboxylic groups.

As used herein, the term “iron chelators” is used to refer to molecules that are able to bind iron in a selective way because they have higher affinity for iron than for any other metal ions. They typically contain oxygen, nitrogen or sulfur-donor atoms that form coordinate bonds with bound iron. The donor atoms of the ligand affect the preference of the chelator for either the Fe(II) or Fe(III) oxidation states.

As used herein, the “Debye length” (also called Debye radius), named after Peter Debye, is a measure of a charge carrier's net electrostatic effect in a solution and how far its electrostatic effect persists. A Debye sphere is a volume whose radius is the Debye length. With each Debye length, charges are increasingly electrically screened. Every Debye-length X_{D}, the electric potential will decrease in magnitude by 1/e. Specifically, in physiological solution environments, which are relevant to many important biological, medical, and diagnostic applications, the short screening length, <1 nm, reduces the field produced by charged biomolecules at a detector (e.g. a FET) surface and thus makes real-time label-free detection difficult. This short screening length is also called “Debye limit” or “Debye screening limitation”.

The term “detectable label”, as used in the present invention, refers to a molecule with a physical property or biochemical activity that is analyzable by a detector via the label's physical property or the label's catalyzed activity. Non-limiting examples of detectable labels include fluorescent labels, fluorogenic labels, dyes, colorimetric labels, radioactive labels, luminescent labels, chemiluminescent labels, and enzymatic labels. A fluorogenic label may be a substrate for an enzymatic or chemical reaction that emits light following the reaction. In some embodiments, the fluorogenic label is an enzyme or a chemical reactant that causes a substrate to fluoresce following an enzymatic or chemical reaction. A colorimetric label may be a substrate for an enzymatic or chemical reaction that changes color following the reaction. In some embodiments, the colorimetric label is an enzyme or a chemical reactant that causes a substrate to change color following an enzymatic or chemical reaction. The detectable label may also be a molecule that can be detected by, e.g., a Surface Acoustic Wave (SAW) device or a Field Effect Transistor (FET). The detectable label may be contained within the lipid vesicle or displayed on the surface of the lipid vesicle. In some embodiments, the detectable label may be the lipids forming the lipid vesicle.

The term “detergent,” as used herein, refers to a surfactant or a mixture of surfactants. Examples of surfactants include, but not limited to, anionic surfactants, non-ionic surfactants, cationic surfactants, amphoteric surfactants (including betaine surfactants and zwitterionic surfactants) and mixtures thereof.

The term “epitope,” as used herein, refers to an antigen determinant, which is the part of the antigen that is recognized by the antibody. Epitopes usually consist of surface groupings of molecules, such as amino acids or sugar side chains, and usually have specific three-dimensional structural characteristics, as well as specific charge characteristics. The epitope may comprise amino acid residues directly involved in the binding and other amino acid residues, which are not directly involved in the binding, such as amino acid residues which are effectively blocked by the antigen binding peptide. In some embodiments, the antibody conjugate specifically recognizes and binds to a different epitope on the marker than the capture molecule.

The term “dielectric material”, as used herein, refers to an electrical insulator that can be polarized by an applied electric field. When a dielectric material is placed in an electric field, electric charges do not flow through the material as they do in an electrical conductor but only slightly shift from their average equilibrium positions causing dielectric polarization. A perfect dielectric material is a material with zero electrical conductivity, exhibiting only a displacement current. Therefore, it stores and returns electrical energy as if it were an ideal capacitor. The dielectric constant of a material, also called the permittivity of a material, represents the ability of a material to concentrate electrostatic lines of flux. In more practical terms, it represents the ability of a material to store electrical energy in the presence of an electric field.

The term “Field Effect Transistor” (FET), as used herein, refers to a transistor that uses an electric field to control the electrical behavior of the device. FET consists of three electrodes: source, drain, and gate. The positive gate voltage attracts electrons from the bulk to the surface of the substrate. A sufficient number of electrons induced form a thin n-channel by electrically bridging the source and drain. Otherwise, when a specific molecular recognition occurs on the gate, the FET detects the change of charge density at the interface by an electrostatic interaction with the electrons in the n-channel A skilled person in the art will be able to determine the materials coated on the surface of the gate insulator of the FET. The FET may be a chelator-coated FET, such as those described in U.S. Provisional Application No. 62/718,632, U.S. Provisional Application No. 62/886,759 and PCT/US2019/046568, each of which is incorporated by reference herein in its entirety.

The term “lipid vesicle,” as used in the present application, refers to spherical bilayers which are comprised of one or more lipids. As used herein, the lipid vesicles of the invention may also be referred to as “liposomes”. The type, number and ratio of lipids may vary with the proviso that collectively they form spherical bilayers or vesicles. The lipids may be isolated from a naturally occurring source or they may be synthesized apart from any naturally occurring source. There are three main types of lipid vesicles: (1) a multilamellar vesicle (MLV), with several lamellar phase lipid bilayers; (2) a small unilamellar liposome vesicle (SUV) with one lipid bilayer and a diameter typically ranging between 15-30 nm and (3) a large unilamellar vesicle (LUV) with one lipid bilayer and a diameter typically ranging between 100-300 nm or larger. Lipid vesicles may be disrupted by contacting them with, e.g., a detergent. Optionally, the detergent is a non-ionic detergent.

The terms “marker” and “analyte” as used herein are used interchangeably herein and refer to one or more molecules that are differentially present in a sample and that are indicators of the presence of an event, condition or process. The term “biomarker”, as used herein, refers to one or more biological molecules that are differentially released into a biological fluid by any means (including secretion or by leakage through the cell membrane). The term “biomarker” refers to a distinctive biological or biologically derived indicator of a process, event or condition. Analyte biomarkers can be used in methods of diagnosis, e.g. clinical screening, and prognosis assessment and in monitoring the results of therapy, identifying patients most likely to respond to a particular therapeutic treatment, drug screening and development. Diagnostically useful biomarkers are identified using measured levels of a single biomarker obtained from a statistically significant number of disease-negative and disease-positive subjects in a population and establishing a mean and a standard deviation for the disease negative and positive states.

A “microfluidics device” or “biochip,” as used herein, refers to a device or system that has channels and/or chambers that are generally fabricated on the micron or submicron scale. The typical channels or chambers have at least one cross-sectional dimension in the range of about 0.1 microns to about 500 microns. Optionally, the cross-sectional dimension is in the range of 10 to 500, of 20 to 500, of 40 to 500, of 80 to 500, of 100 to 500, of 200 to 500, of 300 to 500, or of 400 to 500. Optionally, the cross-sectional dimension is in the range of about 0.1 to about 400 microns, of 10 to 400, of 20 to 400, of 40 to 400, of 80 to 400, of 100 to 400, of 200 to 400, of 300 to 400. Optionally, the cross-sectional dimension is in the range of about 0.1 to about 300 microns, of 10 to 300, of 20 to 300, of 40 to 300, of 80 to 300, of 100 to 300, of 200 to 300 microns. The microfluidic device comprises multiple “microfluidic channel blocks,” with fluid flow between said blocks being selectively operable. In the context of the present application, a “block” may be defined as a discrete area on the device having a microfluidic channel with a long path within a confined space.

The term “microorganism,” as used herein, is a living organism of microscopic or ultramicroscopic size, that is too small to be seen with the naked eye and which can be a single celled or a colony of cells. The microorganism can be a bacterium, an archaeon, an alga, a protozoan, a fungus or a virus.

As used herein, “protease cleavage site” refers to an amino acid sequence that is recognized and cleaved by a protease. Examples of protease cleavage sites can be selected from the group of thrombin, plasmin, Factor Xa, trypsin, pepsin, Lys-N, Glu-C, caspase, Asp-N or Arg-C.

The term “sample,” as used herein, can refer to a fluid wherein the markers or biomarkers are present, or a fluid derived from the specimen into which the markers or biomarkers are initially present. In some embodiments, the sample is a biological sample into which biomarkers are released, or a fluid derived from the biological sample into which biomarkers are initially released. Such derivation may occur either in vivo or in vitro. In some instances, the biological sample is a circulating fluid such as blood or lymph, or a fraction thereof, such as serum or plasma. In other cases, the biological sample remains substantially in a particular locus, for example, synovial fluid, cerebrospinal fluid or interstitial fluid. In still further cases, the biological fluid is an excreted fluid, for example, urine, breast milk, saliva, sweat, tears, mucous, nipple aspirants, semen, vaginal fluid, pre-ejaculate and the like. A biological fluid also refers to a liquid in which cells are cultured in vitro such as a growth medium, or a liquid in which a cell sample is homogenized, such as a buffer. In some cases, the sample is a food sample or an environmental sample, such as a water or a soil sample, which contains markers or molecules to be detected.

The term “scaffold,” as used in the present invention, refers to a solid phase onto which the capture molecule is or can be adsorbed or immobilized. The term “solid phase” means a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase may be a stationary component, such as a surface, a membrane, a tube, a strip, a cuvette or a microtiter plate, or may be a non-stationary component, such as beads and microparticles. A variety of microparticles that allow either non-covalent or covalent attachment of proteins and other substances may be used. Such particles include polymer particles such as polystyrene and poly(methylmethacrylate); gold particles such as gold nanoparticles and gold colloids; and ceramic particles such as silica, glass, and metal oxide particles. See for example Martin, C. R., et al., Analytical Chemistry-News & Features, May 1, 1998, 322A-327A.

The term “Surface Acoustic Wave device” (SAW), as used herein, refer to mass sensors which operate with mechanical acoustic waves as their transduction mechanism and wherein the acoustic wave propagates, guided or unguided, along a single surface of the substrate. Any other Acoustic Wave biosensor can be suitable for use in the present invention, such as Bulk Acoustic Wave (BAW) devices or Acoustic Plate Mode devices (APM), wherein in BAW devices the acoustic wave propagates unguided through the volume of the substrate and in APM devices the waves are guided by reflection from multiple surfaces. The SAW and APM devices can be grouped as Surface Generated Acoustic Wave (SGAW) devices, because both develop acoustic waves generated and detected in the surface of the piezoelectric substrate by means of Interdigital Transducers (IDTs). Examples of SGAW devices are Shear Horizontal Surface Acoustic Wave (SH-SAW), Surface Transverse Wave (STW), Love Wave (LW), Flexural Plate Wave (FPW), Shear Horizontal Acoustic Plate Mode (SH-APM) and Layered Guided Acoustic Plate Mode (LG-APM).

The term “uniform”, as used herein, refers to the lipid vesicles having the same size or substantially the same size. The term “uniform” may also refer to the lipid vesicle membrane comprising only one type of lipid.

### 3. Antibody-Lipid Vesicle Conjugates and Aptamer-Lipid Vesicle Conjugates

In a first aspect, the disclosure of the application provides an antibody conjugate comprising an antibody linked to an amphiphilic lipid vesicle, wherein the vesicle comprises a detectable label. This aspect of the disclosure also provides an aptamer linked to a lipid vesicle. In some embodiments, the aptamer is an oligonucleotide. Optionally, the aptamer comprises DNA residues. In some embodiments, the aptamer comprises RNA residues. Optionally, the oligonucleotide is single-stranded.

The type, number and ratio of lipids in the vesicle may vary. In some embodiments, the vesicles are spherical. The lipids may be isolated from a naturally occurring source or they may be synthesized apart from any naturally occurring source. In some embodiments, the liposome or lipid vesicle is a multilamellar vesicle (MLV), with several lamellar phase lipid bilayers. In another embodiment, the liposome or lipid vesicle is a small unilamellar liposome vesicle (SUV) with one lipid bilayer and a diameter typically ranging between 15-30 nm. In another embodiment, the liposome or lipid vesicle is a large unilamellar vesicle (LUV) with one lipid bilayer and a diameter typically ranging between 100-200 nm or larger.

In some embodiments, the lipid vesicle comprises an amphipathic or amphiphilic lipid, which have a hydrophilic portion and a hydrophobic portion, for example hydrophilic head and a hydrophobic tail. The hydrophobic portion typically orients into a hydrophobic phase (e.g., within the bilayer), while the hydrophilic portion typically orients toward the aqueous phase (e.g., outside the bilayer, and possibly between adjacent apposed bilayer surfaces). The hydrophilic portion may comprise polar or charged groups such as carbohydrates, phosphate, carboxylic, sulfate, amino, sulihydryl, nitro, hydroxy and other like groups. The hydrophobic portion may comprise apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

In some embodiments, the lipids are phospholipids. Phospholipids include, without limitation, phosphatidic acid, phosphatidylcholine, phosphatidylethanolamine, phosphatidylglycerol, phosphatidylinositol or phosphoinositides, phosphatidylserine, and combinations thereof.

The lipids may be anionic and neutral (including zwitterionic and polar) lipids including anionic and neutral phospholipids. Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include, without limitation, dioleoylphosphatidylcholine (DOPC), dimyristoylphosphatidylcholine (DMPC), and dioleoylphosphatidylserine (DOPS). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids and combinations thereof.

Collectively, anionic and neutral lipids are referred to herein as non-cationic lipids. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol and combinations thereof. In one embodiment, the lipid is a phospholipid. In some embodiments, the phospholipid is a phosphatidylcholine. In some embodiments, the lipid vesicle comprises lecithin.

In some embodiments, the lipid vesicles are uniform in size. Optionally, the sizes of the lipid vesicles are not uniform in size. In some embodiments, all lipids present in the lipid vesicle are phospholipids. In other embodiments, all lipids present in the lipid vesicle are phosphatidylcholine. In some embodiments, the lipids present in the lipid vesicle comprise a combination of different phospholipids, such as a combination of neutral, anionic or cationic lipids. For example, the lipid vesicle may comprise a mixture comprising cholesterol and other lipids.

The number of lipid bilayers in each vesicle may vary, with a typical range of at least 1 to about 50, or at least 1 to about 25, or at least 1 to about 15, or at least 1 to about 10, or at least 1 to about 5 lipid bilayers. In a preferred embodiment, the number of lipid bilayers is from about 1 to about 10. In some embodiments, the vesicle comprises 1 lipid bilayer. Optionally, the vesicle comprises 2 lipid bilayers.

The vesicle may comprise 3 lipid bilayers. In some embodiments, the vesicle comprises 4 lipid bilayers. Optionally, the vesicle comprises 5 lipid bilayers. The vesicle may comprise 6 lipid bilayers. In some embodiments, the vesicle comprises 7 lipid bilayers. Optionally, the vesicle comprises 8 lipid bilayers. The vesicle may comprise 9 lipid bilayers. In some embodiments, the vesicle comprises 10 lipid bilayers.

The diameter of the vesicles may vary. In some embodiments, the vesicles will have a diameter ranging from about 20 to about 100 nm, from about 25 to about 50 nm, from 100 to about 500 nm, from about 200 to about 500, from about 300 to about 500, from about 400 to about 500, from about 100 to about 400 nm, from about 200 to about 400, from about 300 to about 400, from about 100 to about 300 nm, from about 150 to about 300 nm, from about 200 to about 300 nm, or from about 100 to 1000 nm.

It will be understood that, in any preparation of vesicles, there will be certain heterogeneity between the vesicles relating to vesicle diameter, number of lipid bilayers, etc.

Methods for the synthesis of the lipid vesicles are known in the art. An exemplary synthesis for MLVs is as follows: Lipids and optionally other bilayer components are combined to form a homogenous mixture. This may occur through a drying step in which the lipids are dried to form a lipid film. The lipids are then combined (e.g., rehydrated) with an aqueous solvent. The aqueous solvent may have a pH in the range of about 6 to about 8, including a pH of about 7. Buffers compatible with vesicle fusion are used, typically with low concentrations of salt, such as for example bis-tris propane (BTP) buffer or PBS. The nature of the buffer may impact the length of the incubation. Accordingly, a variety of aqueous buffers may be used provided that a sufficient incubation time is also used.

In some embodiments, an ionic solution is incorporated in the resultant liposomes by including ions in the solvent for rehydration. Vesicles may be broken down to smaller sizes by vigorous mixing to obtain very large multilamellar vesicles. In some embodiments, vesicles may be broken down to smaller sizes by sonication to obtain the smallest possible single-walled vesicles, or alternatively by various means to obtain vesicles of intermediate size and characteristics. The liposomes may be prepared in the presence of a charged solution, such as ions and, therefore, the lipid vesicles will comprise the charged solution (e.g., ionic solution) in their core. In some embodiments, the ionic solution comprises a metal ion. Optionally, the metal ion is a divalent or trivalent ion. The metal ion may be selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion. In some embodiments, the heavy metal ion is selected from the group consisting of As⁺³, Hg⁺², Sb⁺³, and Au⁺. In some embodiments, the ionic solution comprises a cation selected from the group consisting of Ca²⁺, Fe²⁺, Fe³⁺, Mg²⁺, Mn²⁺, Cu²⁺, Cu³⁺, Zn²⁺, Ni²⁺, Co²⁺, As⁺³, Hg⁺², Sb⁺³, and Au⁺. Optionally, the ionic solution comprises Ca²⁺.

The resultant MLVs may then be incubated with a crosslinker, and preferably a membrane-permeable crosslinker. The nature of the crosslinker will vary depending on the nature of the reactive groups being linked together. For example, a dithiol-containing crosslinker such as DTT or (1,4-Di-[3′-(2′-pyridyldithio)-propionamido]butane) may be used to crosslink MLVs comprised of maleimide functionalized lipids (or other functionalized lipid bilayer components), or diazide crosslinkers could be used to crosslink alkyne headgroup lipids via “click” chemistry. These various incubations are all carried out under aqueous conditions at a pH in the range of about 6 to about 8, or about 6.5 to about 7.5, or at about 7. The crosslinking step may be performed at room temperature (e.g., 20-25° C.) or at a higher temperature, including for example up to or higher than 37° C.

The resultant crosslinked lipid vesicles are then collected (e.g., by centrifugation or other pelleting means), washed with water or other aqueous buffer and then PEGylated (if needed) on their outermost or external surface by incubation with a thiol-PEG. The PEG may be of any size, including but not limited to 0.1-10 kDa, 0.5-5 kDa, or 1-3 kDa.

In other embodiments, the lipid vesicles are loaded with a peptide. In some embodiments, the peptide is bound to an antibody attached to a scaffold and the peptide can be detected by using a second antibody or a different molecule detecting said peptide. For example, the peptide may comprise the TAP tag comprised of two protein A domains and the calmodulin binding peptide separated by a TEV cleavage site. Once released from the lipid vesicle, the peptide may bind to an antibody bound to a scaffold and it may be detected by the binding to calmodulin.

The vesicles may be stored at 4° C. in a buffered solution such as, but not limited to, PBS or they may be lyophilized in the presence of suitable cryopreservants and then stored at −20° C. Suitable cryopreservants include those that include sucrose.

In some embodiments, the SUV are prepared by sonication using, for example, a cup horn, bath, or probe tip sonicator. Optionally, LUV are prepared by a variety of methods. Examples of those methods include extrusion (LUVET or “Large Unilamellar Vesicles prepared by Extrusion Technique”), detergent dialysis (DOV or “Di-Octylglucoside Vesicles”), fusion of SUV (FUV or “Fused Unilamellar Vesicles”), reverse evaporation (REV or “Reverse Evaporation Vesicles”), and ethanol injection.

In another embodiment, the lipid vesicles of the present application are hybrid vesicles resulting from the combined self-assembly of both amphiphilic copolymers and lipids.

Methods for the conjugation or coupling of the lipid vesicle to an antibody or to aptamers are known in the art.

In some embodiments, the aptamer is covalently conjugated to the surface of the lipid vesicle, whose core may be encapsulated with different molecules. Method for the conjugation of aptamers to lipid vesicles include, but are not limited to, conjugation by maleimide, conjugation by utilizing the terminal —COOH group present on liposomes, by incubation with 3′-thiol-5′-FITC, etc.

Antibodies and aptamers can be conjugated (a) directly on the phospholipid head groups of non-PEGylated liposomes; (b) conjugated directly on the phospholipid headgroups of PEGylated liposomes; or (c) conjugated on the free terminus of PEGylated chains. An example of conjugation directly on the lipid head group is the following: the antibody or aptamer to be coupled to the vesicle is modified by reaction with a bifunctional agent which reacts with a free NH₂ group on the antibody and provides a free sulfhydryl group available for attachment to the vesicle. The modified antibody or aptamer, which retains its chemical activity after the modification, is then reacted with the lipid vesicle containing the free sulfhydryl group under conditions such that a S—S bond is formed, thereby covalently linking the antibody or aptamer to the vesicle. Examples of bifunctional agents are selected from a group consisting of N-hydroxysuccinimidyl 3-(2-pyridyldithio) propionate, PDP (3-(2-pyridyldithio)propionate), maleimide, MBP, MCC derivatives and chemical analogs thereof. Other methods to conjugate antibodies or aptamer to liposomes or lipid vesicles comprise the following: (1) conjugation through modified antibody or aptamer using avidin-biotin binding; (2) conjugation through thiol modified antibody or aptamer; (3) conjugation through maleimide modified antibody or aptamer; (4) conjugation through aldehyde modified antibody to a hydrazide modified lipid or aptamer; (5) conjugation through a hydrazide modified antibody to an aldehyde modified lipid or aptamer; (6) conjugation of EDC/NHS activated PEGylated carboxylic acid modified lipid to N-terminus of antibody or aptamer; (7) conjugation of EDC/NHS activated (PEGylated or non-PEGylated) succinyl modified lipid to N-terminus of antibody or aptamer; (8) conjugation of EDC/NHS activated glutaryl modified lipid to N-terminus of antibody (non-PEGylated) or aptamer; (9) conjugation of EDC/NHS activated dodecanoyl modified lipid to N-terminus of antibody (non-PEGylated); (10) conjugation of NHS ester lipid to N-terminus of antibody or aptamer; (11) conjugation of cyanur modified (PEGylated or non-PEGylated) lipid to N-terminus of antibody or aptamer; (12) conjugation through a carboxy group on an EDC/NHS activated antibody or aptamer to an amine modified PEGylated lipid; (13) conjugation through a carboxy group on an EDC/NHS activated antibody or aptamer to a phosphatidylethanolamine (PE) lipid; and (14) conjugation through a carboxy group on an EDC/NHS activated antibody or aptamer to a caproylamine lipid. In some embodiments, the antibodies are incorporated into the lipid vesicles through Protein A/G. Protein A/G is incorporated in the phospholipid structure and serves as an attachment site for the antibody. In some embodiments, the antibody is incorporated into the lipid vesicles through the binding of protein G-coated polystyrene beads to the antibody. The protein G-coated polystyrene beads induced discrete jumps in relative resonance wavelength shift attributable to individual binding events of single beads or bead aggregates. In some embodiments, the antibodies are incorporated into the lipid vesicles through the binding of streptavidin-modified polystyrene beads to biotinylated vesicles. The number of beads bound to each vesicles ring may be determined via scanning electron microscopy (SEM) and plotted versus the net resonance wavelength shift of the corresponding ring.

In one embodiment, the antibody or aptamer is linked to the lipid vesicle by a linker. In another embodiment, the linker is a peptide linker. In some embodiments, the peptide linker has a length ranging between about 5 and 50 amino acids, including from about 10 to 40 amino acids or from about 15 to 35 amino acids or from about 20 to 30 amino acids. In a preferred embodiment, the length ranges between about 5 and 50 amino acids. Optionally, the peptide linker comprises a protease cleavage site.

In some embodiments, the peptide linker comprises a protease cleavage site. Optionally, the protease cleavage sites is selected from the group consisting of thrombin, plasmin, Factor Xa, trypsin, pepsin, Lys-N, Glu-C, caspase, Asp-N or Arg-C.

In some embodiments, the linker is released by the cleavage of a disulfide bond. The cleavage of the disulfide bond may occur via reduction. A variety of reductants may be used. Non-limiting examples of reductants to be used for the cleavage of the disulfide bonds include thiols, such as β-mercaptoethanol (β-ME) or dithiothreitol (DTT). Other reductants that may be used include tris(2-carboxyethyl)phosphine (TCEP) and sodium borohydride.

Methods for encapsulation of ions in lipid vesicles or liposomes are known in the art. Examples of these methods are detailed in McConnell and Kornberg (Biochemistry, 1971, 10 (7), pp 1111-1120), incorporated by reference herein in its entirety, and the references disclosed therein. Commercial sources are also available (such as Avanti Polar Lipids). The confirmation of successful encapsulation can be done by electrochemical methods.

In some embodiments, the lipid vesicle is stable and has no leaks over time, releasing the vesicle content only upon disruption. Optionally, at least around 90% of the detectable label remains in the vesicle over time, such as around 91%, 92%, 93%, 94%, 95%, 96%, 97%, 98%, 99% or around 100% of the detectable label remains in the vesicle. In other embodiments, at least around 90% of the detectable label remains in the vesicle over time, such as for at least one month, for at least two months, for at least three months, for at least four months, for at least five months, for at least six months, for at least seven months, for at least eight months, for at least nine months, for at least ten months, for at least eleven months, for at least twelve months, for at least eighteen months, for at least twenty months or for at least twenty four months.

In some embodiments, the lipid vesicle is soluble in a detergent solution and disruption is performed by adding a detergent. In a preferred embodiment, the detergent is a non-ionic detergent. Non-ionic detergents are known by the skilled person in the art and are typically based on polyoxyethylene or a glycoside. Non-limiting examples of non-ionic detergents include Tween, Triton, Nonidet P40 (NP-40) and the Brij series. In some embodiments, the lipid vesicle is susceptible to disruption. In some embodiments, the disruption is an enzymatic disruption and the disruption is performed by adding an enzyme. Optionally, the disruption is performed by adding a non-detergent chemical. In other embodiments, the disruption is performed by using antimicrobial peptides. Examples of antimicrobial peptides include, but are not limited to melittin, mastoparan, poneratoxin, cecropin, moricin, magainin, dermaseptin, aurein, copsin, cathelicidins, defensins and protegrins.

In some embodiments, the detectable label is selected from the group consisting of a charged solution, a charged particle, a magnetic particle, a metal particle, a luminescent label, a colorimetric substrate, a fluorescent label, an enzymatic label or a radioactive label or a combination thereof. Optionally, the charged solution is an ionic solution. In some embodiments, the ionic solution comprises a metal ion. Optionally, the metal ion is a divalent or trivalent ion. The metal ion may be selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion. In some embodiments, the heavy metal ion is selected from the group consisting of As⁺³, Hg⁺², Sb⁺³, and Au⁺. In some embodiments, the ionic solution comprises a cation selected from the group consisting of Ca²⁺, Fe²⁺, Fe³⁺, Mg²⁺, Mn²⁺, Cu²⁺, Cu³⁺, Zn²⁺, Ni²⁺, Co²⁺, As⁺³, Hg⁺², Sb⁺³, and Au⁺. Optionally, the ionic solution comprises Ca²⁺.

A fluorogenic label may be a substrate for an enzymatic or chemical reaction that fluoresces following the reaction. In some embodiments, the fluorogenic label is an enzyme or a chemical reactant that causes a substrate to fluoresce following an enzymatic or chemical reaction. A luminescent label may be a substrate for an enzymatic or chemical reaction that causes luminescence following the reaction. In some embodiments, the luminescent label is an enzyme or a chemical reactant that causes a substrate to luminesce following an enzymatic or chemical reaction. A colorimetric label may be a substrate for an enzymatic or chemical reaction that changes color following the reaction. In some embodiments, the colorimetric label is an enzyme or a chemical reactant that causes a substrate to change color following an enzymatic or chemical reaction. Examples of fluorescent labels include MDCC (Coumarin), Cy3/Cy5, Fluorescein, Rhodamine, GFP, RFP, Alexa dyes, FITC, TRITC, DyLight fluors, 6-carboxyfluorescein and Qdots. Examples of enzymatic labels include horseradish peroxidase (HRP), alkaline phosphatase (AP), glucose oxidase and β-galactosidase, Formylglycine generating enzyme (FGE), Phosphopantetheinyl transferase (PPTase), Sortase, Transglutaminase, Farnesyl transferase, Biotin ligase and Lipoic acid ligase. Examples of radioactive labels include Phosphorus-32, Phosphorus-33, Hydrogen-3 Carbon-14, Sulfur-35, Yttrium-90, Gallium-68 and Iodine-125.

Examples of methods for detection of the detectable label include fluorescence, luminescence, colorimetry, radioactivity, Surface Acoustic Wave (SAW) or Surface Generated Acoustic Wave (SGAW) and Field Effect Transistor (FET).

In some embodiments, the detectable label is capable of being detected by a Surface Acoustic Wave (SAW) device. Non-limiting examples of labels capable of being detected by a SAW device include a magnetic particle, a metal particle, any particle of 1 pg or greater, a microbe, and a spore.

In some embodiments, the detectable label is capable of being detected by a Field Effect Transistor (FET). Non-limiting examples of labels capable of being detected by a FET device include a charged particle, magnetic particle, a metal particle and a lipid vesicle comprising a charged solution. Optionally, the charged solution is an ionic solution. In some embodiments, the FET is a chelator-coated FET as described in US 62/718,632, U.S. Provisional Application No. 62/886,759 and PCT/US2019/046568, each of which is incorporated by reference herein in its entirety. In some embodiments, the ionic solution comprises a metal ion. Optionally, the metal ion is a divalent or trivalent ion. The metal ion may be selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion. In some embodiments, the heavy metal ion is selected from the group consisting of As⁺³, Hg⁺², Sb⁺³, and Au⁺. In some embodiments, the ionic solution comprises a cation selected from the group consisting of Ca²⁺, Fe²⁺, Fe³⁺, Mg²⁺, Mn²⁺, Cu²⁺, Cu³⁺, Zn²⁺, Ni²⁺, Co²⁺, As⁺³, Hg⁺², Sb⁺³, and Au⁺. Optionally, the ionic solution comprises Ca²⁺.

Optionally, the detectable label is capable of being detected by surface plasmon resonance (SPR). In some embodiments, if a magnetic or metal particle is used as a detectable label, the solutions can be mixed by cycling an electric/magnetic field.

In some embodiments, the detectable label may be contained within the lipid vesicle or displayed on the surface of the lipid vesicle. In some embodiments, the detectable label may be the lipids forming the lipid vesicle.

In some embodiments, the detector is a Surface Acoustic Wave device. Any other Acoustic Wave biosensor may be used in the present disclosure, including Bulk Acoustic Wave (BAW) devices or Acoustic Plate Mode devices (APM). In BAW devices the acoustic wave propagates unguided through the volume of the substrate, and in APM devices the waves are guided by reflection from multiple surfaces. The SAW and APM devices can be combined in Surface Generated Acoustic Wave (SGAW) devices, because both develop acoustic waves generated and detected in the surface of the piezoelectric substrate by means of Interdigital Transducers (IDTs).

Examples of SGAW devices, that can be used to detect the detectable label, are Shear Horizontal Surface Acoustic Wave (SH-SAW), Surface Transverse Wave (STW), Love Wave (LW), Flexural Plate Wave (FPW), Shear Horizontal Acoustic Plate Mode (SH-APM) and Layered Guided Acoustic Plate Mode (LG-APM).

The input port of a SGAW sensor, comprised of metal electrodes or Interdigital Transducers (IDTs) deposited or photodesigned on an optically polished surface of a piezoelectric crystal, launches a mechanical acoustic wave into the piezoelectric material due to the inverse piezoelectric phenomenon and the acoustic wave propagates through the substrate. SAW or SGAW techniques require one binding component to be immobilized on a transducer surface, while the other binding component in buffer solution is flowed over the transducer surface. A binding interaction is detected using an acoustic method that measures small changes in the phase and amplitude of the acoustic waves that travel through the transducer sensor surface. The output signals, corresponding to changes in the phase and amplitude of waves, give information about the pure mass loading, intrinsic properties of bound materials, and viscoelastic effects such as conformational changes in protein structures, protein-protein complexes, and the internal structure of layers. These changes can be detected with network analyzers, vector voltmeters or more simple electronics, such as oscillators. These sensors offer a method for not only detection but also quantification of binding events because of being capable of measuring real-time quantitative binding affinities (k_{D}) and kinetic constants (kₒₙ and k_{off}) of biological complexes and also concentrations of target analytes. The dimensions and physical properties of the piezoelectric substrate determine the optimal resonant frequency for the transmission of the acoustic wave and will be determined by the skilled person in the art.

The immobilization of biomolecules on the solid substrate of the transducer surface helps to ensure biosensor performance, because of its role in specificity, sensitivity, reproducibility and recycling ability. In some embodiments, covalent binding is used to attach biomolecules to the transducer surface. Covalent immobilization provides a reproducible, durable and stable attachment to the substrate against physico-chemical variations in the aqueous microenvironment. Self-assembled monolayer (SAM) technology provides the best results in covalent binding and allows the generation of monomolecular layers of biological molecules on a variety of substrates. Gold surfaces allow the use of functionalized thiols, whereas SiO2 surfaces enable the use of various silanes. Both methods produce monolayers of active groups for the subsequent coupling of biomolecules onto the transducer surface. A skilled person will determine and develop the immobilization method for every combination of biological sample and sensor surface.

In some embodiments, the antibody conjugate or aptamer conjugate of the present disclosure is used in a diagnostic marker analysis system for the detection of markers or microorganisms, providing a sensitive, specific, and robust system with small sample consumption.

### 4. Methods for Detecting Markers in a Sample

In a second aspect, the disclosure relates to a method of detecting a marker in a sample. In some embodiments, the method comprises:
(a) contacting the sample with a capture molecule that binds the marker, wherein the capture molecule is affixed to a scaffold or is capable of being affixed to a scaffold,
(b) contacting the marker with a composition comprising the antibody conjugate according to the first aspect of the invention, wherein antibody conjugate binds to a different epitope on the marker than the capture molecule;
(c) contacting the marker-bound antibody conjugate with a composition capable of releasing the detectable label from the amphiphilic lipid vesicle on the antibody conjugate; and
(d) detecting the detectable label.

In some embodiments, the method comprises:
(a) contacting the sample with a capture molecule that binds the marker, wherein the capture molecule is affixed to a scaffold or is capable of being affixed to a scaffold,
(b) contacting the marker with a composition comprising the aptamer conjugate according to the first aspect of the invention, wherein the aptamer conjugate binds to a different region on the marker than the capture molecule;
(c) contacting the marker-bound aptamer conjugate with a composition capable of releasing the detectable label from the amphiphilic lipid vesicle on the aptamer conjugate; and
(d) detecting the detectable label.

In some embodiments, the method for the detection of markers or microorganisms of the present disclosure provides a sensitive, specific, and robust sensing detection system with small sample consumption.

In some embodiments, the sample is a biological sample. Optionally, the biological sample comprises one or more markers. The sample may be a biological sample into which one or more biomarkers are released, or a fluid derived from the biological sample into which one or more biomarkers are initially released. Such derivation may occur either in vivo or in vitro. In some instances, the biological sample is a circulating fluid such as blood or lymph, or a fraction thereof, such as serum or plasma. In other embodiments, the biological sample remains substantially in a particular locus, for example, synovial fluid, cerebrospinal fluid or interstitial fluid. Optionally, the biological sample is an excreted fluid, for example, urine, breast milk, saliva, sweat, tears, mucous, nipple aspirants, semen, vaginal fluid, pre-ejaculate and the like. A biological sample may also comprise a liquid in which cells are cultured in vitro such as a growth medium, or a liquid in which a cell sample is homogenized, such as a buffer. In some embodiments, the sample is a food sample. Optionally, the sample is an environmental sample, such as a water or a soil sample, which contains markers or molecules to be detected. The sample may contain an allergen or a microorganism. In some embodiments, the microorganism is selected from the group of a bacterium, a fungus, an archaeon, an alga, a protozoan and a virus.

In some embodiments, the marker is a biomarker, an environmental marker, an allergen, or a microorganism (e.g. a bacterium, a fungus, an archaeon, an alga, a protozoan and a virus). Examples of markers which can be detected according to the methods of the present disclosure include proteins, lipids, lipoproteins, glycoproteins, nucleic acids (including circulating nucleic acids), carbohydrates, lipopolysaccharides, small molecule metabolites, and fragments thereof. Typically, the presence and/or the concentration of a biomarker (or biomarkers, or pattern or patterns of biomarkers) in a sample is discriminatory between physiological and pathological states of the cells from which they are released.

In some embodiments, the marker or biomarker to be detected by the method of the invention is present in the sample at a concentration that cannot be detected without signal amplification and a step of amplification is required in order to detect the marker in the sample or improve the signal detection. In some embodiments, the amplification is performed by encapsulation of ions, peptides, etc. in the lipid vesicles, which are detected once the vesicles are disrupted and, as a consequence, the signal is amplified.

In some embodiments, the capture molecule is affixed to the scaffold. Optionally, the capture molecule is capable of being affixed to the scaffold. For example, the capture molecule may be linked to a magnetic bead or a metallic particle, which will bind the scaffold upon the application of an electric current or a magnetic field. In such embodiments, the cycling of the electric current or the magnetic field can be used to mix the composition of the method. In some embodiments, the scaffold is a “solid phase” to which the capture molecule is affixed or is capable of being affixed. Non-limiting examples of solid phases that may be used in the present disclosure include particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. In some embodiments, the solid phase is a stationary component, such as a surface, a membrane, a tube, a strip, a cuvette or a microtiter plate, or may be a non-stationary component, such as beads and microparticles. Microparticles that allow either non-covalent or covalent attachment of proteins and other substances may be used. Non-limiting examples of microparticles include polymer particles such as polystyrene and poly(methylmethacrylate); gold particles such as gold nanoparticles and gold colloids; and ceramic particles such as silica, glass, and metal oxide particles. In some embodiments, the capture molecule is a capture antibody. Optionally, the capture molecule is a capture aptamer.

In some embodiments, the scaffold, to which the capture molecule is affixed or is capable of being affixed, is a detector for the detectable label. Non-limiting examples of methods for detection of the detectable label or detectors include Surface Acoustic Wave (SAW) or Surface Generated Acoustic Wave (SGAW) and Field Effect Transistor (FET). Said methods have already been defined and explained in detail in the present application.

In some embodiments, the scaffold, to which the capture molecule is affixed, is adjacent to a detector for the detectable label. Optionally, if the detectable label is not adjacent to the detector, the present method comprises an additional step of transporting the detectable label to a detector for the detectable label. Non-limiting examples of means for transporting the detectable label to a detector include channels, pumps, pressure-driven flow, electrokinetically-driven flow and other known by the skilled in the art.

The conditions suitable for the contact and the binding of the capture molecule and the marker; of the antibody conjugate and the marker; or of the aptamer conjugate and the marker are known by the skilled in the art, who can choose the right conditions, such as temperature, buffer and pH.

The affinity binding of the capture molecule to the marker present in the sample or of the capture molecule bound to the marker and the antibody conjugate or aptamer conjugate of the invention, as referred in the present application, may be measured by the dissociation constant or K_{D}. In some embodiments, the antibody binds to its antigen or the aptamer binds to its target with an affinity corresponding to a K_{D} of about 10⁻¹⁰ M or less, e.g. 10⁻⁷ M or less, such as about 10⁻⁸ M or less, such as about 10⁻⁹ M or less, about 10⁻¹⁰ M or less, or about 10⁻¹¹ M or even less. K_{D} values are measured by techniques known by the skilled in the art, such as, for example ELISA, surface plasmon resonance (SPR), fluorescence anisotropy, Bio-Layer Interferometry, typically using OCTET® technology (Octet QKe system, ForteBio) or a KinExA® (Kinetic Exclusion Assay) assay.

In some embodiments, the capture molecule is an antibody. Methods for derivatizing antibodies to permit disposition onto surfaces of the scaffold are known in the art. Non-limiting examples for conjugating antibodies to a surface include, but are not limited to, (1) crosslinking through sulihydryl-reactive groups by reacting a thiol group of the antibody (such as the ones present in Cysteine) with any sulfhydryl-reactive chemical groups, including haloacetyls, maleimides, aziridines, acryloyls, arylating agents, vinylsulfones, pyridyl disulfides, TNB-thiols and disulfide reducing agents; (2) 1-Pyrenebutyric acid N-hydroxysuccinimide ester (pyrene-NHS). Pyrenes are hydrophobic polycyclic aromatics that bind avidly to the the FET substrate and they do not adversely affect the electrical properties of the substrate (see, for example, Stefansson et al. Journal of Nanotechnology, vol. 2012, Article ID 490175, 2012), incorporated herein by reference in its entirety; and (3) through biomolecules such as biotin/streptavidin. In some embodiments, the capture antibody or derivatized capture antibody is at least partially interposed on a surface containing gold.

In some embodiments, the conditions capable of releasing the detectable label from the lipid vesicle comprise a composition. In some embodiments, the agent or composition capable of releasing the detectable marker is able to disrupt the lipid vesicle. Examples of agents or compositions that can be used to release the detectable marker from the lipid vesicle include, but are not limited to, a low-pH composition, a composition comprising a detergent, a composition comprising an enzyme, a composition comprising a non-detergent chemical compound, and a composition comprising bovine serum or a combination thereof. Non-limiting examples of conditions used to disrupt or release the lipid vesicles are freeze-thaw cycles, heat, light or ultrasound. The releasing agent/condition to be used will be dependent on the type of lipid vesicle used.

In some embodiments, the method comprises contacting the marker-bound antibody conjugate or aptamer conjugate with a composition comprising a detergent, wherein the detergent is present in a sufficient concentration to release the detectable label from the lipid vesicle on the antibody conjugate or aptamer conjugate. Examples of surfactants include, but are not limited to, anionic surfactants, non-ionic surfactants, cationic surfactants, amphoteric surfactants (including betaine surfactants and zwitterionic surfactants) and mixtures thereof.

Examples of suitable anionic surfactants include, but are not limited to, compounds in classes known as alkyl sulfates, alkyl ether sulfates, sulfate esters of an alkylphenoxy polyoxyethylene ethanol, alpha-olefin sulfonates, betaalkyloxy alkane sulfonates, alkyl arylsulfonates, alkyl carbonates, alkyl ether carboxylates, fatty acids, alkyl sulfosuccinates, alkyl ether sulfosuccinates, alkyl sarcosinates, alkyl phosphates, alkyl ether phosphates, octoxynol phosphates, nonoxynol phosphates, alkyl taurates, fatty methyl taurides, sulfated monoglycerides, fatty acid amido polyoxyethylene sulfates, acyl amino acids, and acyl isethionates and mixtures thereof. In one embodiment, the anionic surfactant is present in the composition as a neutralized salt such as sodium salts, potassium salts, ammonium salts, lithium salts, alkyl ammonium salts, or hydroxyalkyl ammonium salts. Preferred anionic surfactants are alkyl sulfates, alkyl ether sulfates, alkyl phosphates, acyl amino acid salts such as N-acyl-L-glutamate, α-olefin sulfonates, alkyl sarcosinates, alkyl benzene sulfonates, acyl isethionates, alkyl sulfosuccinates, acyl methyl taurides, and mixtures thereof.

Non-ionic detergents are known by the skilled person in the art and are typically based on polyoxyethylene or a glycoside. Examples of suitable non-ionic surfactants include, but are not limited to, Tween series (such as polysorbate 20 or poly sorbate 80), Triton series (such as Triton X-100 or Polyoxyethylene octyl phenyl ether), Brij series (such as Brij 35 or Polyethylene glycol dodecyl ether), Nonidet P40 (NP-40), long chain alkyl glucosides having alkyl groups containing about 8 carbon atoms to about 22 carbon atoms, coconut fatty acid monoethanolamides such as cocamide MEA, coconut fatty acid diethanolamides, and mixtures thereof.

Examples of suitable cationic surfactants include, but are not limited to, quaternary ammonium surfactants and quaternary amine surfactants that are not only positively charged at the pH of the composition, which generally is about pH 10 or lower, but also are soluble in the composition. Preferred cationic surfactants include, but are not limited to, the n-acylamidopropyl dimethylamine oxides such as cocamidopropylamine oxide.

Examples of suitable amphoteric surfactants include, alkyl amphocarboxylates, alkyl betaines, amidoalkylbetaines, amidoalkylsultaines, alkyl amphophosphates, alkyl phosphobetaines, amido-alkyl phosposphobetaines, alkyl pyrophosphobetaines, amido-alkyl pyrophosposphobetaines, carboxyalkyl alkyl polyamines, and mixtures thereof. Preferred amphoteric surfactants include amidoalkylbetaines such as cocamidopropyl betaine available commercially from Goldschmidt Chemical Corporation of Hopewell, Va. under the tradename “Tegobetaine L-7”; alkyl amphocarboxylates having from about 8 carbon atoms to about 18 carbon atoms in the alkyl group such as Sodium Cocoamphopropionate available commercially from Mona Industries Inc. of Paterson, N.J. under the tradename “Monateric CA-35”.

In some embodiments, the conditions capable of releasing the detectable label comprise a composition. In some embodiments, the composition capable of releasing the detectable label comprises an enzyme and the lipid vesicle is susceptible to enzymatic disruption. Optionally, the disruption is performed by adding a non-detergent chemical compound.

In some embodiments, the detectable label comprises an ionic solution. In some embodiments, the detectable label comprises an ion, e.g. a metal ion. Non-limiting examples of metal ions include iron ions, copper ions, cobalt ions, manganese ions, chromium ions, nickel ions, zinc ions, cadmium ions, molybdenum ions, lead ions, and the like. In any of the methods disclosed herein, the metal ion being detected is, optionally, selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion (e.g., As⁺³, Hg⁺², Sb⁺³, and Au⁺). Preferably, the metal ions to be detected are divalent and trivalent ions.

In some embodiments, when the detectable label comprises a metal ion, the method further comprises contacting the released metal ions with a metal ion chelator or metal ion derivatized chelator located at or near the detector. In some embodiments, the metal ion chelator is attached to the surface of the detector. In some embodiments, the detector is a chelator-coated FET, such as those described in U.S. Provisional Application No. 62/718,632, U.S. Provisional Application No. 62/886,759 and PCT/US2019/046568, each of which is incorporated by reference herein in its entirety.

In some embodiments, chelating agents of metallic ions include chelating agents of Ca²⁺, Fe²⁺, Fe³⁺, Mg²⁺, Mn²⁺, Cu²⁺, Cu³⁺, Zn²⁺, Ni²⁺, Co²⁺, heavy metal ions (e.g., As⁺³, Hg⁺², Sb⁺³, and Au⁺), and the like. It is within the skill of the art to select a chelating agent or derivatized chelating agent that will bind or complex with a particular ion of interest. See, e.g., Bers D. M., MacLeod K. T. (1988) Calcium Chelators and Calcium Ionophores. In: Baker P. F. (eds) Calcium in Drug Actions. Handbook of Experimental Pharmacology, vol 83. Springer, Berlin, Heidelberg; Hatcher, H C. et al. Future Med Chem. 2009 December; 1(9): 10.4155; Sheth, S., Curr Opin Hematol 2014, 21:179; Missy P. et al. Hum Exp Toxicol., 2000, vol. 19(8): 448-456; Sigma Aldrich, BioUltra Reagents: Chelators (available at https://www.sigmaaldrich.com/life-science/metabolomics/bioultra-reagents/chelators.html); Santa Cruz Biotechnology Chelators (available at https://www.scbt.com/scbt/browse/chelators/_/N-lazot5l); Lawson M K, et al. Curr Pharmacol Rep (2016) 2:271-280; Radford and Lippard, Curr Opin Chem Biol. 2013 April; 17(2): 129-136; Chaitman, M. et al., P T. 2016 January; 41(1): 43-50, each of which is incorporated herein in its entirety.

In some embodiments, the chelating agent or derivatized chelating agent selectively binds a metal ion. Preferably, the chelating agent or derivatized chelating agent selectively binds the metal ion contained within the lipid vesicle of a detection molecule, such as a detection antibody. Optionally, the chelating agent or derivatized chelating agent binds several metal ions. The chelating agent or derivatized chelating agent may preferentially bind one metal ion, but still bind other metal ions. In some embodiments, the chelator is a custom designed chelator.

In some embodiments, the chelator is selected from the group consisting of 1,1,1-Trifluoroacetylacetone; 1,4,7-Trimethyl-1,4,7-triazacyclononane; 2,2′-Bipyrimidine; Acetylacetone; Alizarin; Amidoxime; Amidoxime group; Aminoethylethanolamine; Aminomethylphosphonic acid; Aminopolycarboxylic acid; ATMP; BAPTA; Bathocuproine; BDTH2; Benzotriazole; Bidentate; Bipyridine; 2,2′-Bipyridine; Bis(dicyclohexylphosphino)ethane; 1,2-Bis(dimethylarsino)benzene; 1,2-Bis(dimethylphosphino)ethane; 1,2-Bis(diphenylphosphino)ethane; Calixarene; Carcerand; Catechol; Cavitand; Chelating resin; Chelex 100; Citrate; Citric acid; Clathrochelate; Corrole; Cryptand; 2.2.2-Cryptand; Cyclam; Cyclen; Cyclodextrin; Deferasirox; Deferiprone; Deferoxamine; Denticity; Dexrazoxane; Diacetyl monoxime; Trans-1,2-Diaminocyclohexane; 1,2-Diaminopropane; 1,5-Diaza-3,7-diphosphacyclooctanes; 1,4-Diazacycloheptane; Dibenzoylmethane; Diethylenetriamine; Diglyme; 2,3-Dihydroxybenzoic acid; Dimercaprol; 2,3-Dimercapto-1-propanesulfonic acid; Dimercaptosuccinic acid; 1,1-Dimethylethylenediamine; 1,2-Dimethylethylenediamine; Dimethylglyoxime; DIOP; Diphenylethylenediamine; 1,5-Dithiacyclooctane; Domoic acid; DOTA; DOTA-TATE; DTPMP; EDDHA; EDDS; EDTA; EDTMP; EGTA; 1,2-Ethanedithiol; Ethylenediamine; Ethylenediaminediacetic acid; Ethylenediaminetetraacetic acid; Etidronic acid; Fluo-4; Fura-2; Gallic acid; Gluconic acid; Glutamic acid; Glyoxal-bis(mesitylimine); Glyphosate; Hexafluoroacetylacetone; Homocitric acid; Iminodiacetic acid; Indo-1; Isosaccharinic acid; Kainic acid; Ligand; Malic acid; Metal acetylacetonates; Metal dithiolene complex; Metallacrown; Nitrilotriacetic acid; Oxalic acid; Oxime; Pendetide; Penicillamine; Pentetic acid; Phanephos; Phenanthroline; O-Phenylenediamine; Phosphonate; Phthalocyanine; Phytochelatin; Picolinic acid; Polyaspartic acid; Porphine; Porphyrin; 3-Pyridylnicotinamide; 4-Pyridylnicotinamide; Pyrogallol; Salicylic acid; Sarcophagine; Sodium citrate; Sodium diethyldithiocarbamate; Sodium polyaspartate; Terpyridine; Tetramethylethylenediamine; Tetraphenylporphyrin; Thenoyltrifluoro acetone; Thioglycolic acid; TPEN; 1,4,7-Triazacyclononane; Tributyl phosphate; Tridentate; Triethylenetetramine; Triphos; Trisodium citrate; 1,4,7-Trithiacyclononane; and TTFA and derivatives thereof.

In some embodiments, the metal ion is Ca²⁺. The chelator may be a Ca²⁺ chelator or derivatized therefrom. Optionally, the chelator or the derivatized chelator for Ca²⁺ is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium salt (HEDTA); Nitrilotriacetic acid (NTA); BAPTA; 5,5′-dimethyl BAPTA (such as tetrapotassium salt); DMNP-EDTA; INDO 1 (such as pentapotassium salt); FURA-2 (such as pentapotassium salt); FURA 2/AM; MAPTAM; FLUO 3 (such as pentaammonium salt); Tetraacetoxymethyl Bis(2-aminoethyl) Ether N,N,N′,N′-Tetraacetic Acid; 2-{(carboxymethyl) 2-trimethylaminoethyl amino} acetic acid and salts of such agents, as well as free acids, derivatives and combinations thereof. Preferably, the chelator or the derivatized chelator for Ca²⁺ is EGTA or a derivative thereof.

Methods to determine the calcium binding affinity of EGTA are known in the art. A non-limiting example of such method is the Bers method (Bers D M. Am J Physiol. 1982; 242(5):C404-8), incorporated by reference herein in its entirety, wherein free Ca²⁺ in Ca-EGTA solutions are measured with a Ca electrode, bound Ca is calculated, and Scatchard and double-reciprocal plots are resolved for the total EGTA and the apparent Ca-EGTA association constant (Kₐₚₚ) in the solutions used. The free Ca²⁺ is then recalculated using the determined parameters, giving a more accurate knowledge of the free Ca²⁺ in these solutions and providing an accurate calibration curve for the Ca electrode. This method allows determination of free Ca²⁺, Kₐₚₚ, and total EGTA in the actual solutions used regardless of pH, temperature, or ionic strength.

In some embodiments, the metal ion is Fe²⁺ or Fe³⁺. The chelator may be a Fe²⁺ or Fe³⁺ chelator or derivatized therefrom. Optionally, the chelator or derivatized chelator for Fe²⁺ or Fe³⁺ is selected from the group consisting of deferasirox; deferiprone; deferoxamine; desferrioxamine; desferrithiocin[2-(3-hydroxypyridin-2-yl)-4-methyl-4,5-dihyrothiazole-4-carboxylic acid; clioquinol; O-trensox (Tris-N-(2-aminoethyl-[8-hydroxyquinoline-5-sulfonato-7-carboxamido] amine); tachpyr (N,N′,N″-tris(2-pyridylmethyl)-cis,cis-1,3,5-triamino-cyclohexane); dexrazoxane; triapine (3-aminopyridine-2-carboxaldehyde thiosemicarbazone); pyridoxal isonicotinoyl hydrazone; di-2-pyridylketone thiosemicarbazone series; flavan-3-ol; curcumin; apocynin; kolaviron; floranol; baicalein; baicalin; Ligusticum wallichi Francha (ligustrazine); quercetin; epigallocatechin gallate; theaflavin; phytic acid; genistein (5,7,4′-tri-hydroxyisoflavone); EDTA; NTA; HBED, o-Phenanthroline monohydrate; Pyridoxal Isonicotinoyl Hydrazone, 2,2prime-Dipyridyl, (S) 1 (p Bromoacetamidobenzyl)ethylenediaminetetraacetic Acid, (S) 1 (4 Aminoxyacetamidobenzyl)ethylenediaminetetraacetic Acid; Lipoic Acid and salts of such agents, as well as the free acids, derivatives thereof and combinations thereof.

In some embodiments, the metal ion is Mg²⁺. The chelator may be a Mg²⁺ chelator or derivatized therefrom. Optionally, the chelator or the derivatized chelator for Mg²⁺ is selected from the group consisting of EDTA, EGTA, HEDTA, NTA and salts of such agents, as well as the free acids, derivatives thereof and combinations thereof.

In some embodiments, the metal ion is Mn²⁺. The chelator may be a Mn²⁺ chelator or derivatized therefrom. Optionally, the chelator or the derivatized chelator for Mn²⁺ is selected from the group consisting of EDTA; EGTA; HEDTA; NTA; triethylenetetramine-N,N,N′,N″,N′″,N′″-hexaacetic acid (TTHA); para-aminosalicylic acid (PAS), 1,2-cyclohexylenedinitrilotetraacetic acid (CDTA), nitrilotriacetic acid (NAS), diethylenetriaminepentaacetic acid (DTPA); DPTA-OH; HBED; and salts of such agents, as well as the free acids, derivatives thereof and combinations thereof.

In some embodiments, the metal ion is Cu²⁺ or Cu³⁺. The chelator may be a Cu²⁺ or Cu³⁺ chelator or derivatized therefrom. Optionally, the chelator or the derivatized chelator for Cu²⁺ or Cu³⁺ is selected from the group consisting of EDTA; NTA; D-Penicillamine (DPA); Tetraethylenetetraamine (TETA); clioquinol; glutamic acid; lipoic acid; and salts of such agents, as well as the free acids, derivatives thereof and combinations thereof.

In some embodiments, the metal ion is Zn²⁺. The chelator may be a Zn²⁺ chelator or derivatized therefrom. Optionally, the chelator or the derivatized chelator for Zn²⁺ is selected from the group consisting of ADAMTS-5 Inhibitor; N,N,N′,N′-tetrakis(2-pyridinylmethyl)-1,2-ethanediamine (TPEN); EDPA; 1,2-bis(o-aminophenoxy)ethane-N,N,N′,N′-tetraacetic acid (BAPTA); CaEDTA; EDTA; EGTA; Tricine; ZXl; 4-{[2-(bis-pyridin-2-ylmethylamino)ethylamino]methyl}phenyl)methanesulfonic acid (DPESA); [4-({[2-(bis-pyridin-2-ylmethylamino)ethyl]pyridin-2-ylmethylamino}-methyl)phenyl]methanesulfonic acid (TPESA); and derivatives thereof.

In some embodiments, the metal ion is Ni²⁺. The chelator may be a Ni²⁺ chelator or derivatized therefrom. Optionally, the chelator or the derivatized chelator for Ni²⁺ is selected from the group consisting of citrate, malate, histidine, EDTA, sodium diethyldithiocarbamate (Dithiocarb), dimethyldithiocarbamate, diisopropyl, morpholine-I-dithiocarbamate, N,N′-ethylene-bis-dithiocarbamate, 2-2(oxo-1-imidazo-lidyl) ethyldithiocarbamate, dithiocarbamate, tetraethylthiuram (Antabuse), d-penicillamine, dimercaprol (BAL), N-methyl formamide, 8-Hydroxyquinoline-Cyclodextrin Conjugate, glutamic acid and salts of such agents, as well as the free acids, derivatives thereof and combinations thereof. In some embodiments the chelator or derivatized chelator for Ni²⁺ is a nickel binding protein. See, e.g., Sudan R J J, et al. (2015) Ab Initio Coordination Chemistry for Nickel Chelation Motifs. PLoS ONE 10(5): e0126787. doi:10.1371/journal.pone.0126787, incorporated by reference herein in its entirety.

In some embodiments, the metal ion is Co²⁺. The chelator may be a Co²⁺ chelator or derivatized therefrom. Optionally, the chelator or the derivatized chelator for Co²⁺ is selected from the group consisting of L-cysteine; L-methionine; N-acetyl-cysteine; EDTA; sodium 2,3-dimercaptopropane sulfonate (DMPS); diethylenetriaminepentaacetic acid (DTPA); 2,3-dimercaptosuccinic acid (DMSA); dimercaprol; 8-Hydroxyquinoline-Cyclodextrin Conjugate; glutamic acid; deferasirox; desferrioxamine; deferiprone; and salts of such agents, as well as the free acids, derivatives thereof and combinations thereof.

In some embodiments, the metal ion is a heavy metal ion. Optionally, the heavy metal ion is selected from the group consisting of As⁺³, Hg⁺², Sb⁺³, and Au⁺. The chelator may be a heavy metal ion chelator or derivatized therefrom. In some embodiments, the chelator or the derivatized chelator for the heavy metal ion is selected from the group consisting of Dimercaprol (2,3-dimercapto-1-propanol); Sodium 2,3 dimercaptopropanesulfonate monohydrate; 2,3-Dimercapto-1-propanesulfonic acid sodium salt; Dimercaptosuccinic acid; Penicillamine; Lipoic Acid; and salts of such agents, as well as the free acids, derivatives thereof and combinations thereof. In some embodiments, the chelator or derivatized chelator for Au⁺ comprises an SH group. Optionally, the chelator or derivatized chelator for Hg²⁺ comprises an SH group.

Methods for adding a thiol group to the chelator are known in the art. Non-limiting examples include, but are not limited, to: (a) Potassium thioacetate was added into a solution of 1,4-diioidobutane to afford the corresponding thioester. The thioester is added to a dilute solution of K₄EGTA, resulting in the formation of the mono-functionalized thioester-K₃EGTA. Thioester-K₃EGTA then reacts with KOH followed by neutralization with HCl to afford EGTA-SH; (b) Addition of 2-aminoethane-1-thiol to a solution of protected EGTA; and (c) Reaction of EGTA with 1-pyrenebutyric acid to form a thioester.

Methods for derivatizing chelators to permit disposition onto surfaces, such as scaffolds and detectors of microfluidics devices are known in the art. For example, pyrenes are known to adsorb to carbon nanotube (CNT) surfaces through π-π interactions. By reacting a chelator, such as EGTA, with 1-pyrenebutyric acid, to form the corresponding thioester, the chelator can be adsorbed to the carbon nanotube surface. Additionally, azide chemistry has been demonstrated to be a powerful means to covalently modify carbon nanotubes. Specifically, diazonium salts react with the surface of carbon nanotube surfaces to generate C—C bonds. Through the derivatization of the chelating agent with a diazonium salt, the chelator can be attached to the surface of the device. In some embodiments, the diazonium salt is 4-(2,5-dioxo-2,5-dihydro-1H-pyrrol-1-yl)benzenediazonium.

In some embodiments, the method of the disclosure further comprises a step of washing or removing the unbound material present in the sample, once the marker is bound to the capture molecule or antibody in the scaffold. In some embodiments, the method for detecting a marker in a sample further comprises the step of removing unbound marker between the step of contacting the sample with the capture molecule and the step of contacting the marker with the antibody conjugate. Optionally, the method for detecting a marker in a sample further comprises the step of removing unbound marker between the step of contacting the sample with the capture molecule and the step of contacting the marker with the aptamer conjugate. Optionally, the method comprises one or more washing steps between the step of contacting the sample with the capture molecule and the step of contacting the marker with the antibody conjugate or with the aptamer conjugate, wherein the markers not bound to the capture molecule are removed. In some embodiments, the method further comprises one or more filtering steps between the step of contacting the sample with the capture molecule and the step of contacting the marker with the antibody conjugate or with the aptamer conjugate, wherein the marker not bound to the capture molecule are removed.

In some embodiments, the method for detecting a marker in a sample further comprises the step of removing unbound capture molecule before the step of contacting the capture molecule with the releasing agent. Optionally, the method comprises one or more washing steps between the step of contacting the marker with the antibody conjugate or with the aptamer conjugate and the step of contacting marker-bound antibody conjugate or marker-bound aptamer conjugate with a releasing agent, wherein the antibody conjugate molecules or the aptamer conjugate molecules not bound to the marker are removed. In some embodiments, the method further comprises one or more filtering steps between the step of contacting the marker with the antibody conjugate or with the aptamer conjugate and the step of contacting marker-bound antibody conjugate or the marker-bound aptamer conjugate with a releasing agent, wherein the antibody conjugate or aptamer conjugate molecules not bound to the marker are removed.

Optionally, the method for detecting a marker in a sample is performed on a microfluidics device. Optionally, the method for detecting a marker in a sample is performed using a field effect transistor (FET) or a sensor comprising a FET.

A third aspect of the present disclosure relates to a method of detecting one of a plurality of markers in a sample. In some embodiments, the method comprises:
(a) contacting the sample with a first capture molecule and a second capture molecule, wherein the first capture molecule is affixed to a first scaffold or is capable of being affixed to the first scaffold and binds a first marker, wherein the second capture molecule is affixed to a second scaffold or is capable of being affixed to the second scaffold and binds a second marker, wherein the first marker is different from the second marker;
(b) contacting the first marker with a composition comprising a first antibody conjugate, wherein the first antibody conjugate is an antibody conjugate according to the first aspect of the invention, and wherein the first antibody conjugate recognizes a different epitope on the first marker than the first capture molecule;
(c) contacting the second marker with a composition comprising a second antibody conjugate, wherein the second antibody conjugate is an antibody conjugate according to the first aspect of the invention, and wherein the second antibody conjugate recognizes a different epitope on the second marker than the second capture molecule;
(d) contacting the first marker-bound first antibody conjugate with a composition capable of releasing a first detectable label from the amphiphilic lipid vesicle on the first antibody conjugate;
(e) contacting the second marker-bound second antibody conjugate with a composition capable of releasing a second detectable label from the amphiphilic lipid vesicle on the second antibody conjugate;
(f) performing a first detection step to detect the first detectable label; and
(g) performing a second detection step to detect the second detectable label.

In some embodiments, the method comprises:
(a) contacting the sample with a first capture molecule and a second capture molecule, wherein the first capture molecule is affixed to a first scaffold or is capable of being affixed to the first scaffold and binds a first marker, wherein the second capture molecule is affixed to a second scaffold or is capable of being affixed to the second scaffold and binds a second marker, wherein the first marker is different from the second marker;
(b) contacting the first marker with a composition comprising a first aptamer conjugate, wherein the first aptamer conjugate is an aptamer conjugate according to the first aspect of the invention, and wherein the first aptamer conjugate recognizes a different region on the first marker than the first capture molecule;
(c) contacting the second marker with a composition comprising a second aptamer conjugate, wherein the second aptamer conjugate is an aptamer conjugate according to the first aspect of the invention, and wherein the second aptamer conjugate recognizes a different region on the second marker than the second capture molecule;
(d) contacting the first marker-bound first aptamer conjugate with a composition capable of releasing a first detectable label from the amphiphilic lipid vesicle on the first aptamer conjugate;
(e) contacting the second marker-bound second aptamer conjugate with a composition capable of releasing a second detectable label from the amphiphilic lipid vesicle on the second aptamer conjugate;
(f) performing a first detection step to detect the first detectable label; and
(g) performing a second detection step to detect the second detectable label.

The skilled artisan would recognize that in any of the methods of detecting one of a plurality of markers in a sample, a mixture of antibody conjugates and aptamer conjugates could be used. In such embodiments, the method comprises:
(a) contacting the sample with a first capture molecule and a second capture molecule, wherein the first capture molecule is affixed to a first scaffold or is capable of being affixed to the first scaffold and binds a first marker, wherein the second capture molecule is affixed to a second scaffold or is capable of being affixed to the second scaffold and binds a second marker, wherein the first marker is different from the second marker;
(b) contacting the first marker with a composition comprising a first aptamer conjugate, wherein the first aptamer conjugate is an aptamer conjugate according to the first aspect of the invention, and wherein the first aptamer conjugate recognizes a different region on the first marker than the first capture molecule;
(c) contacting the second marker with a composition comprising a second antibody conjugate, wherein the second antibody conjugate is an antibody conjugate according to the first aspect of the invention, and wherein the second antibody conjugate recognizes a different epitope on the second marker than the second capture molecule;
(d) contacting the first marker-bound first aptamer conjugate with a composition capable of releasing a first detectable label from the amphiphilic lipid vesicle on the first aptamer conjugate;
(e) contacting the second marker-bound second antibody conjugate with a composition capable of releasing a second detectable label from the amphiphilic lipid vesicle on the second antibody conjugate;
(f) performing a first detection step to detect the first detectable label; and
(g) performing a second detection step to detect the second detectable label.

In other embodiments, the method comprises:
(a) contacting the sample with a first capture molecule and a second molecule antibody, wherein the first capture molecule is affixed to a first scaffold or is capable of being affixed to the first scaffold and binds a first marker, wherein the second capture molecule is affixed to a second scaffold or is capable of being affixed to the second scaffold and binds a second marker, wherein the first marker is different from the second marker;
(b) contacting the first marker with a composition comprising a first antibody conjugate, wherein the first antibody conjugate is an antibody conjugate according to the first aspect of the invention, and wherein the first antibody conjugate recognizes a different epitope on the first marker than the first capture molecule;
(c) contacting the second marker with a composition comprising a second aptamer conjugate, wherein the second aptamer conjugate is an aptamer conjugate according to the first aspect of the invention, and wherein the second aptamer conjugate recognizes a different region on the second marker than the second capture molecule;
(d) contacting the first marker-bound first antibody conjugate with a composition capable of releasing a first detectable label from the amphiphilic lipid vesicle on the first aptamer conjugate;
(e) contacting the second marker-bound second aptamer conjugate with a composition capable of releasing a second detectable label from the amphiphilic lipid vesicle on the second antibody conjugate;
(f) performing a first detection step to detect the first detectable label; and
(g) performing a second detection step to detect the second detectable label.

In the method of the detection of one of a plurality of markers, the definitions and examples detailed above for the method of detection of one marker apply herein. In some embodiments, the first capture molecule is a capture antibody. Optionally, the first capture molecule is a capture aptamer. In some embodiments, the second capture molecule is a capture antibody. Optionally, the second capture molecule is a capture aptamer. In some embodiments, the first marker is contacted with the first capture molecule before the first marker is contacted with the first antibody conjugate. Optionally, the first marker is contacted with the first capture molecule after the first marker is contacted with the first antibody conjugate. In some embodiments, the first marker is contacted with the first capture molecule before the first marker is contacted with the first aptamer conjugate. Optionally, the first marker is contacted with the first capture molecule after the first marker is contacted with the first aptamer conjugate. In some embodiments, the second marker is contacted with the second capture molecule before the second marker is contacted with the second antibody conjugate. Optionally, the second marker is contacted with the second capture molecule after the second marker is contacted with the second antibody conjugate. In some embodiments, the second marker is contacted with the second capture molecule before the second marker is contacted with the second aptamer conjugate. Optionally, the second marker is contacted with the second capture molecule after the second marker is contacted with the second aptamer conjugate.

In some embodiments, the method of detecting one of a plurality of markers comprises contacting the sample with at least two or more capture molecules which specifically recognize and bind to two or more different markers, wherein each of the capture molecules are affixed to a scaffold or are capable of being affixed to a scaffold (i.e. first and second scaffolds). In some embodiments, the at least two or more capture molecules comprise capture antibodies. In some embodiments, the at least two or more capture molecules comprise capture aptamers. Optionally, the at least two or more capture molecules comprise at least one capture antibody and at least one capture aptamer.

In some embodiments, the first scaffold is a detector for the first detectable label. Optionally, the second scaffold is a detector for the second detectable label. In some embodiments, the first scaffold is adjacent to a detector for the first detectable label. Optionally, the second scaffold is adjacent to a detector for the second detectable label

In some embodiments, the first capture molecule is bound to a magnetic bead or a metallic bead. Optionally, the first capture molecule binds to the first or second scaffold upon the cycling of an electric current. In some embodiments, the second capture molecule is bound to a magnetic bead or a metallic bead. Optionally, the second capture molecule binds to the first or second scaffold upon the cycling of an electric current.

In some embodiments, the method of detecting one of a plurality of markers is used for the detection of more than two markers and in such embodiments, more than two antibody conjugates or aptamer conjugates are used. A skilled person in the art will know how to adapt the present method accordingly to more than two antibody or aptamer conjugates. For example, the step of contacting the sample with a capture molecule may comprise a different capture molecule for each marker being detected in the sample. Optionally, the step of contacting the sample with a capture molecule comprises a different capture molecule for each marker of interest that may be in the sample. Similarly, the step of contacting the capture molecule-marker with the antibody conjugate or aptamer conjugate may be performed with a capture molecule specific for each marker being detected in the sample. Optionally, the step of contacting the capture molecule-marker with the antibody conjugate or aptamer conjugate may be performed with a capture molecule specific for each marker of interest that may be in the sample. Additionally, the step of releasing the detectable label and the step of detection of the detectable label may each be repeated for each marker being detected in the sample. Optionally, the step of releasing the detectable label and the step of detection of the detectable label are each repeated for each marker being detected in the sample.

In some embodiments, the method further comprises the step of transporting the first detectable label to a detector for the first detectable label, prior to the detection of the first detectable marker step. Optionally, the method further comprises the step of transporting the second detectable label to a detector for the second detectable label, prior to the detection of the second detectable marker step.

In some embodiments, the method of detecting one of a plurality of markers further includes one or more washing steps. Optionally, the washing steps can be substituted by filtering steps. In some embodiments, the method of the disclosure further comprises a step of washing or removing the unbound material present in the sample, once the marker is bound to the capture molecule or antibody in the scaffold. In some embodiments, the method further comprises the step of removing unbound marker(s) between the step of contacting the marker(s) with capture molecule (first, second and/or subsequent) and contacting the marker with the respective capture molecule. Optionally, the method further comprises the step of washing the capture molecule (first, second and/or subsequent) bound to the marker(s). The method may further comprise the step of washing the capture molecule (first, second and/or subsequent) bound to the marker(s) prior to contacting the capture molecule-bound marker with the respective antibody or aptamer conjugate(s) (first, second and/or subsequent). The method further may comprise the step of filtering the capture molecule (first, second and/or subsequent) bound to the marker(s) to remove unbound marker(s). Optionally, the method further comprises the step of filtering the capture molecule (first, second and/or subsequent) bound to the marker(s) to remove unbound marker(s) prior to contacting the capture molecule-bound marker with the respective antibody or aptamer conjugate(s) (first, second and/or subsequent)

In some embodiments, the method further comprises the step of removing unbound antibody or aptamer conjugate before contacting the marker-bound antibody or aptamer conjugate with the conditions or composition capable of releasing a respective detectable label. Optionally, the method further comprises the step of washing the antibody conjugate(s) or aptamer conjugate(s) (first, second and/or subsequent) bound to the marker(s), before contacting the marker-bound antibody or aptamer conjugate with the conditions or composition capable of releasing the respective detectable label(s).

In some embodiments, the first detectable label and the second detectable label are different. Optionally, any subsequent detectable label is different from the first detectable label, the second detectable label, and any other subsequent detectable label. In such embodiments, the marker is detected by detecting the detectable label associated with that marker in the method.

In some embodiments, the first detectable label and the second detectable label are the same. Optionally, any subsequent detectable label is the same as the first and second detectable labels. In such embodiments, the marker is detected by detecting the detectable label in the first detection step, in the second detection step and/or in any subsequent detection steps. The first and second detection steps may be performed sequentially. Optionally, the first, second and subsequent detection steps are performed sequentially. In some embodiments, the first and second detection steps are performed in different channels. Optionally, the first, second and subsequent detection steps are performed in different channels.

In some embodiments, the first detectable label comprises an ionic solution. In some embodiments, the second detectable label comprises an ionic solution. In some embodiments, the first detectable label comprises an ion, e.g. a metal ion. In some embodiments, the second detectable label comprises an ion, e.g. a metal ion. In some embodiments, the first and the second detectable label comprise a metal ion.

Optionally, the ionic solution of the first and second detectable label comprise the same ion. Optionally, the ionic solution of the first detectable label comprises a different ion than the ionic solution of the second detectable label. Non-limiting examples of metal ions include iron ions, copper ions, cobalt ions, manganese ions, chromium ions, nickel ions, zinc ions, cadmium ions, molybdenum ions, lead ions, and the like. In any of the methods disclosed herein, the metal ion being detected is, optionally, selected from the group consisting of Ca²⁺, Fe²⁺, Fe³⁺, Mg²⁺, Mn²⁺, Cu²⁺, Cu³⁺, Zⁿ²⁺, Ni²⁺, Co²⁺ and a heavy metal ion (e.g., As⁺³, Hg⁺², Sb⁺³, and Au⁺). Preferably, the metal ions to be detected are divalent and trivalent ions. Optionally, the ionic solution of the first detectable label comprises Ca²⁺. In some embodiment, the ionic solution of the second detectable label comprises Ca²⁺.

In some embodiments, when the first detectable label comprises a metal ion, the method further comprises contacting the released metal ions with a metal ion chelator or metal ion derivatized chelator for the first detectable marker located at or near the detector for the first detectable marker. In some embodiments, the metal ion chelator or metal ion derivatized chelator for the first detectable marker is disposed upon the surface of the detector for the first detectable marker. In some embodiments, the detector for the first detectable marker is a chelator-coated FET. The metal ion chelator or metal ion derivatized chelator for the first detectable marker may be disposed upon a scaffold near the detector for the first detectable marker. Optionally, the metal ion chelator or metal ion derivatized chelator for the first detectable marker is one of the metal ion chelators described supra or derivatized therefrom.

In some embodiments, when the second detectable label comprises a metal ion, the method further comprises contacting the released metal ions with a metal ion chelator or metal ion derivatized chelator for the second detectable marker located at or near the detector for the second detectable marker. In some embodiments, the metal ion chelator or metal ion derivatized chelator for the second detectable marker is disposed upon the surface of the detector for the second detectable marker. In some embodiments, the detector for the second detectable marker is a chelator-coated FET. The metal ion chelator or metal ion derivatized chelator for the second detectable marker may be disposed upon a scaffold near the detector for the second detectable marker. Optionally, the metal ion chelator or metal ion derivatized chelator for the second detectable marker is one of the metal ion chelators described supra or derivatized therefrom. In some embodiments, the released metal ions from the first and second detectable labels are the same ions. Optionally, the released metal ions from the first detectable label are different from the released metal ions of the second detectable label.

In some embodiments, the conditions or composition capable of releasing the first detectable label is the same as the conditions or composition capable of releasing the second detectable label. Optionally, the conditions or compositions capable of releasing any subsequent detectable labels are the same as the conditions or compositions capable of releasing the first and second detectable labels.

In some embodiments, the conditions or composition capable of releasing the first detectable label is different from the conditions or composition capable of releasing the second detectable label. Optionally, the conditions or composition capable of releasing any subsequent detectable label is different from the composition capable of releasing the first detectable label, the conditions or composition capable of releasing the second detectable label, and the conditions or composition capable of releasing any other subsequent detectable label.

In some embodiments, the method of detecting one of a plurality of markers is performed on a microfluidics device. Optionally, the first antibody or aptamer conjugate is released from a first channel in the microfluidics device. The second antibody or aptamer conjugate may be released from a second channel in the microfluidics device. In some embodiments, the release of the first antibody or aptamer conjugate from the first channel and the first detection step occur before the release of the second antibody or aptamer conjugate from the second channel and before the second detection step. In methods detecting more than two markers, the subsequent antibody or aptamer conjugates may be released from different channels of the microfluidics device than the first antibody or aptamer conjugate, the second antibody or aptamer conjugate and any other subsequent antibody or aptamer conjugate. In some embodiments, the first, second, and any subsequent antibody or aptamer conjugates are released from the same channel in the microfluidics device, but at different times. Optionally, the first, second and any subsequent antibody or aptamer conjugates are released form the same channel of the microfluidics device at the same time.

In some embodiments, the method comprises the following steps in a microfluidics device:
(a) providing a plurality of antibody conjugates, each comprising a lipid vesicle containing a concentration of ions and an antibody specific for a marker;
(b) conjugating the antibody conjugate with the marker;
(c) separating the marker-bound antibody conjugates from unbound antibody conjugates;
(d) immobilizing the marker-bound antibody conjugates to a scaffold in a testing chamber of the microfluidics device using antibodies (e.g., capture antibodies) specific for the marker and attached to the scaffold to create an ELISA-like sandwich; (e) disposing a wash buffer to remove any unbound antibody conjugates that have accumulated or unattached marker-bound antibody conjugates in the testing chamber, such that the only effective source of lipid vesicles in the testing chamber is immobilized marker-bound antibody conjugates;
(f) disrupting the lipid vesicles of the immobilized marker-bound antibody conjugates to release the concentration of ions into a buffer;
(g) providing an electrode isolated and covered with pure water as a continuous reference value subtracted from the measured conductivity, impedance or resistivity of the buffer to establish a delta value;
(h) providing the delta value to a microcontroller for analysis to generate a time coefficient (τ) for use in establishing reaction kinetics (k+/−); and
(i) the step of measuring conductivity, impedance or resistivity of the buffer to determine the presence and/or extent of conjugation of the marker to the liposomes includes the steps of applying a measuring signal to a first electrode disposed in the buffer, sensing current in a second electrode disposed in the buffer according a magnitude of ions released from the marker sandwich into the buffer, and amplifying and/or signal conditioning the sensed current for output to a detector.

In some embodiments, the method comprises the following steps in a microfluidics device:
(a) providing a maker in a testing chamber of the microfluidics device;
(b) immobilizing the marker to a scaffold in the testing chamber of the microfluidics device using antibodies (e.g., capture antibodies) specific for the marker and attached to the scaffold;
(c) introducing a plurality of antibody conjugates, each comprising a lipid vesicle containing a concentration of ions and an antibody specific for the marker, into the testing chamber;
(d) conjugating the antibody conjugate with the immobilized marker to form an ELISA-like sandwich;
(e) disposing a wash buffer to remove any unbound antibody conjugates that have accumulated or unattached marker-bound antibody conjugates in the testing chamber, such that the only effective source of lipid vesicles in the testing chamber is immobilized marker-bound antibody conjugates;
(f) disrupting the lipid vesicles of the immobilized marker-bound antibody conjugates to release the concentration of ions into a buffer;
(g) providing an electrode isolated and covered with pure water as a continuous reference value subtracted from the measured conductivity, impedance or resistivity of the buffer to establish a delta value;
(h) providing the delta value to a microcontroller for analysis to generate a time coefficient (τ) for use in establishing reaction kinetics (k+/−); and
(i) the step of measuring conductivity, impedance or resistivity of the buffer to determine the presence and/or extent of conjugation of the marker to the liposomes includes the steps of applying a measuring signal to a first electrode disposed in the buffer, sensing current in a second electrode disposed in the buffer according a magnitude of ions released from the marker sandwich into the buffer, and amplifying and/or signal conditioning the sensed current for output to a detector.

In some embodiments, the method comprises performing convection enhanced delivery by recirculating a buffer including the antibody conjugates multiple times through a fluidic circuit including the testing chamber to reduce time required to saturate the capture antibodies from diffusive timescales to convective timescales. Optionally, the method comprises performing convection enhanced delivery by recirculating a buffer including the marker multiple times through a fluidic circuit including the testing chamber to reduce time required to saturate the capture antibodies from diffusive timescales to convective timescales.

In some embodiments, the method further comprises disposing a wash buffer to remove any unbound antibody conjugates that have accumulated or unattached marker-bound antibody conjugates that have accumulated in the testing chamber, such that the only effective source of lipid vesicles in the testing chamber is the immobilized (e.g. capture-antibody-bound) marker-bound antibody conjugate.

In some embodiments, the marker-bound antibody conjugates are circulated in a fluidic circuit and further comprise disposing a wash buffer to remove any unattached marker-bound antibody conjugates that have accumulated in the fluidic circuit.

In some embodiments, the marker-bound antibody conjugate are circulated in a fluidic circuit and further comprising disposing a wash buffer to remove any unbound antibody conjugates that have accumulated in the fluidic circuit, before disrupting the liposome vesicles of the immobilized marker-bound antibody conjugate to release the concentration of ions or cations into a buffer, so that false positives from non-specific bound solvent vesicles are not measured.

In some embodiments, the disrupting step comprises introducing a liposome-disrupting solution into the testing chamber. Optionally, the liposome-disrupting solution comprises a detergent. In some embodiments, the liposome-disrupting solution comprises a liposome-disrupting enzyme

Another aspect included in the present disclosure is a method of improving a limit of detection (LOD) in a microfluidics device. In some embodiments, the method of improving LOD in a microfluidics device comprises:
(a) providing a plurality of antibody conjugates, each comprising a lipid vesicle containing a concentration of ions or cations;
(b) conjugating the antibody conjugates with a selected marker;
(c) disposing the marker-bound antibody conjugates to a testing chamber;
(d) separating the marker-bound antibody conjugates from unbound antibody conjugates;
(e) disrupting the lipid vesicles of the marker-bound antibody conjugates to release the concentration of ions or cations into a buffer; and
(f) measuring conductivity, impedance or resistivity of the buffer to determine the presence and/or extent of conjugation of the marker to the liposomes.

In some embodiments, the method of improving LOD in a microfluidics device comprises:
(a) disposing a marker into a testing chamber in the microfluidics device;
(b) immobilizing the marker using antibodies (e.g., capture antibodies) specific for the marker and affixed to a scaffold in the testing chamber;
(c) providing a plurality of antibody conjugates, each comprising a lipid vesicle containing a concentration of ions or cations and an antibody specific for the maker, into the testing chamber;
(d) conjugating the antibody conjugates with the marker to form an ELISA-like sandwich;
(e) separating the marker-bound antibody conjugates from unbound antibody conjugates;
(f) disrupting the lipid vesicles of the marker-bound antibody conjugates to release the concentration of ions or cations into a buffer; and
(g) measuring conductivity, impedance or resistivity of the buffer to determine the presence and/or extent of conjugation of the marker to the liposomes.

In some embodiments, the step of separating the marker-bound antibody conjugates from unbound antibody conjugates comprises attaching the marker-bound antibody conjugates to an immobilizing surface in the testing chamber.

Optionally, the step of separating the marker-bound antibody conjugates from the unbound antibody conjugates comprises attaching the marker-bound antibody conjugates to a scaffold. In some embodiments, the step of attaching the marker-bound antibody conjugates to an immobilizing surface in the testing chamber comprises attaching the marker-bound antibody conjugates to the immobilizing surface in the testing chamber by using a capture antibody attached to the scaffold (e.g., the immobilizing surface).

In some embodiments, the method further comprises performing convection enhanced delivery by recirculating a buffer including the antibody conjugates multiple times through a fluidic circuit including the testing chamber to reduce time required to saturate the capture antibodies from diffusive timescales to convective timescales. Optionally, the method further comprises performing convection enhanced delivery by recirculating a buffer including the marker multiple times through a fluidic circuit including the testing chamber to reduce time required to saturate the capture antibodies from diffusive timescales to convective timescales.

In some embodiments, the method further comprises disposing a wash buffer to remove any unbound antibody conjugates that have accumulated or unattached marker-bound antibody conjugates that have accumulated in the testing chamber, such that the only effective source of lipid vesicles in the testing chamber is the immobilized (e.g. capture-antibody-bound) marker-bound antibody conjugate.

In some embodiments, the marker-bound antibody conjugates are circulated in a fluidic circuit and further comprise disposing a wash buffer to remove any unattached marker-bound antibody conjugates that have accumulated in the fluidic circuit.

In some embodiments, the marker-bound antibody conjugate are circulated in a fluidic circuit and further comprising disposing a wash buffer to remove any unbound antibody conjugates that have accumulated in the fluidic circuit, before disrupting the liposome vesicles of the immobilized marker-bound antibody conjugate to release the concentration of ions or cations into a buffer, so that false positives from non-specific bound solvent vesicles are not measured.

In some embodiments, the disrupting step comprises introducing a liposome-disrupting solution into the testing chamber. Optionally, the liposome-disrupting solution comprises a detergent. In some embodiments, the liposome-disrupting solution comprises a liposome-disrupting enzyme

In some embodiments, the method further comprises providing an electrode isolated and covered with pure water as a continuous reference value subtracted from the measured conductivity, impedance or resistivity of the buffer to establish a delta value.

In some embodiments, the method further comprises providing the delta value to a microcontroller for analysis to generate a time coefficient (τ) for use in establishing reaction kinetics (k+/−).

In some embodiments, the method further comprises a step of measuring conductivity, impedance or resistivity of the buffer to determine the presence and/or extent of conjugation of the marker to the liposomes comprises applying a measuring signal to a first electrode disposed in the buffer, sensing current in a second electrode disposed in the buffer according a magnitude of ions released from the marker

ELISA sandwich into the buffer, and amplifying and/or signal conditioning the sensed current for output to a detector.

### 5. Microfluidics Devices for Detecting Markers

In a fourth aspect, the present disclosure relates to a microfluidics device comprising:
(a) means for receiving a sample;
(b) a capture molecule, wherein the capture molecule is affixed to a scaffold or is capable of binding to the scaffold and binds a marker in the sample;
(c) means for contacting the sample with the capture molecule;
(d) means contacting the marker with a composition comprising an antibody conjugate, wherein the antibody conjugate is an antibody conjugate according to the first aspect of the invention, and wherein the antibody conjugate binds to a different epitope of the marker than the capture molecule;
(e) means for contacting the marker-bound antibody conjugate with conditions capable of releasing a detectable label from the amphiphilic lipid vesicle on the antibody conjugate; and;
(f) means for detecting the detectable label.

In some embodiments, the microfluidics device comprises:
(a) means for receiving a sample;
(b) a capture molecule, wherein the capture molecule is affixed to a scaffold or is capable of binding to the scaffold and binds a marker in the sample;
(c) means for contacting the sample with the capture molecule;
(d) means contacting the marker with a composition comprising an aptamer conjugate, wherein the aptamer conjugate is an aptamer conjugate according to the first aspect of the invention, and wherein the aptamer conjugate binds to a different region of the marker than the capture molecule;
(e) means for contacting the marker-bound aptamer conjugate with conditions capable of releasing a detectable label from the amphiphilic lipid vesicle on the antibody conjugate; and;
(f) means for detecting the detectable label.

In some embodiments, the channels or chambers of the microfluidics device have at least one cross-sectional dimension in the range of about 0.1 microns to about 500 microns. Optionally, the cross-sectional dimension is in the range of 10 to 500, of 20 to 500, of 40 to 500, of 80 to 500, of 100 to 500, of 200 to 500, of 300 to 500, or of 400 to 500. Optionally, the cross-sectional dimension is in the range of about 0.1 to about 400 microns, of 10 to 400, of 20 to 400, of 40 to 400, of 80 to 400, of 100 to 400, of 200 to 400, of 300 to 400. Optionally, the cross-sectional dimension is in the range of about 0.1 to about 300 microns, of 10 to 300, of 20 to 300, of 40 to 300, of 80 to 300, of 100 to 300, of 200 to 300 microns.

In some embodiments, the microfluidic device comprises multiple microfluidic channel blocks, depending on the number of steps required to perform the method and other methods for the pre-processing of the sample, with fluid flow between said blocks being selectively operable. In some embodiments, said blocks may be arranged sequentially, from a first block to subsequent downstream blocks. Optionally, blocks may form multiple branches of microfluidic channel blocks. As an illustrative example, a first block may be arranged for the purification or extraction of the marker; a second block may be arranged for the contact of the capture molecule with the marker; a third block may be arranged for contacting the marker-bound antibody or aptamer conjugate with a releasing agent or condition; a fourth block may be arranged for detecting the detectable label. In an alternative embodiment, blocks may form multiple branches of microfluidic channel blocks.

In some embodiments, the microfluidics device further comprises a valve for the control of the flow of fluid. Said control include selectively permitting the passage or retention of fluid. The valve may further allow the introduction of new materials to the microfluidic device. Further still, the valve may permit the drainage of waste material from the microfluidic device. In some embodiments, such valves are located between adjacent microfluidic channel blocks, so as to control the flow of fluid between said blocks.

In some embodiments, the microfluidic device further includes means for extracting the sample or means for preparing the sample. In some embodiments, the means for extracting the sample includes a cell disruption step. In certain embodiments, the microfluidics device may comprise means for isolating or purifying the sample after extraction. In some embodiments, the means for extracting or preparing a sample comprises an outlet for connection to another microfluidic device or another channel block.

In some embodiments, the microfluidic device comprises means for receiving a sample. Optionally, the means for receiving the sample is a reservoir or a channel block in the device, wherein the sample is loaded into said sample reservoir, in order to have it tested. In some embodiments, the means for receiving a sample is an input or injection port.

In some embodiments, the microfluidic device further comprises a sample portion that can receive a sample comprising a marker and can place the marker in contact with the detector of the microfluidic device. Optionally, the marker contacts a capture antibody specific for the marker that is interposed on the detector of the microfluidic device (e.g., FET). In some embodiments, the marker is a biomarker, an environmental marker, an allergen, or a microorganism. In some embodiments, the sample is an environmental sample, a food sample, or a sample obtained from a subject.

In some embodiments, the capture molecule is a capture antibody. Optionally, the capture molecule is a capture aptamer. In some embodiments, the microfluidic device comprises means for contacting the sample with the capture molecule. As used herein, “means for contacting the sample with the capture molecule” refers to providing the adequate conditions and parameters, such as antibody concentration, temperature, pH, buffer, etc., so that the capture molecule specifically recognizes and binds to a marker in the sample. It is within the skill in the art to adjust the conditions and parameters based on the capture molecule being used and the marker being detected.

In some embodiments, the microfluidic device comprises means for contacting the marker with a composition comprising the antibody or aptamer conjugate according to the first aspect of the invention, wherein the antibody conjugate binds to a different epitope of the marker than the capture molecule and wherein the aptamer conjugate binds to a different region of the marker than the capture molecule. As used herein, “means contacting the marker with a composition comprising the antibody or aptamer conjugate” refers to providing the adequate conditions and parameters so that the antibody or aptamer conjugate specifically recognizes and binds the marker bound to the capture molecule, such as the right ratio of antibody or aptamer conjugate-marker, temperature, pH, buffer, etc. It is within the skill in the art to adjust the conditions and parameters based on the antibody or aptamer conjugate being used and the marker being detected.

In some embodiments, the microfluidic device comprises means for contacting the antibody or aptamer conjugate bound to the marker with conditions or a composition capable of releasing a detectable label from the lipid vesicle on the antibody conjugate.

The means for detecting the detectable label may be any technique suitable to identify the presence of the detectable label. In some embodiments, the means for detecting the detectable label can be selected from the group consisting of a Surface Acoustic Wave device, a Field Effector Transistor, a fluorescent label detector, an enzymatic label detector, a radioactive label detector and a colorimetric label detector. Optionally, the detectable label is a magnetic particle; a metal particle; a spore; a lipid vesicle comprising charged solution, such as an ionic solution; a fluorescent label; an enzymatic label; a radioactive label; or any other label which is suitable to be detected by the selected technique.

In some embodiments, the scaffold to which the capture molecule is affixed or bound is the detector for the detectable label. Optionally, the scaffold to which the capture molecule is affixed or bound is adjacent to the detector for the detectable label. The microfluidics device may further comprise means for transporting the detectable label to the detector.

In some embodiments, the microfluidic device further comprises means for washing the capture molecule bound to the marker. Optionally, the microfluidics device further comprises means for filtering the capture molecule bound to the marker. In some embodiments, the microfluidics device further comprises means for washing the antibody or aptamer conjugate bound to the marker. Optionally, the microfluidics device further comprises means for filtering the antibody or aptamer conjugate bound to the marker.

In some embodiments, the microfluidic device comprises means for washing or filtering. In some embodiments, the microfluidic device comprises means for washing the immobilized marker, once the marker is bound to the capture molecule in the scaffold, in order to remove unbound materials present in the sample. The means for washing the immobilized marker may comprise moving one or more wash buffers into the channel of the microfluidics device containing the immobilized marker and removing the one or more wash buffers from that channel Optionally, the means for washing the immobilized marker comprises moving the immobilized marker through one or more channels comprising a wash buffer. In some embodiments, the means for washing the immobilized marker comprises moving the immobilized marker into a channel comprising a wash buffer and removing the wash buffer from that channel Optionally, the means for washing the immobilized marker comprises moving the wash buffer into a channel comprising the immobilized marker and removing the immobilized marker from that channel Optionally, multiple steps of binding and washing may be accomplished with the use of magnetic particles or of a matrix (solid phase) through which the specimen and all subsequent mixtures/solutions are passed.

In some embodiments, the microfluidic device comprises means for removing the detection molecule (e.g. detection antibody or detection antibody conjugate) not bound to the marker. In some embodiments, the device comprises means for washing or filtering the detection molecule-bound (e.g. detection antibody-bound) marker. Optionally, the device comprises means for contacting the detection antibody-bound marker with a wash buffer. Preferably, the wash buffer is non-ionic.

In some embodiments, the microfluidic device further comprises means for cycling an electric field or a magnetic field.

In some embodiments, the microfluidic device is incorporated into a cassette or can be part of a cassette. The cassette can further include an electronic device interface electrically coupled to the microfluidic device. The interface may allow the microfluidic device to receive instructions and power from an external or internal source, such as a computing device.

In some embodiments, the microfluidic device includes a power source. The power source may be a battery, a capacitor, a fuel cell, a solar cell. In some embodiments, the microfluidics device comprises a connector to an external power source. The connector may be a USB, USB-c, HDMI, POE, a four-pin connector, a six-pin connector, an eight-pin connector, a twenty-pin connector, and a twenty-four pin connector or any other connector to an external power source. In some embodiments, the microfluidic device comprises means for cycling an electric field or a magnetic field. Examples of means for cycling an electric field or a magnetic field are known by the skilled person in the art.

In some embodiments, the microfluidics device comprises an input/output device. Optionally, the input/output device is a screen, such as a touch screen. In some embodiments, the input/output device comprises a keyboard or one or more switches. The input/output device may comprise a light or a series of lights for signaling the detection of one or more markers or microorganisms. In some embodiments, the microfluidic device includes means for communication of the device with an input/output device. Non-limiting examples of means for communication include a USB port, a USB-c port, an HDMI port, a VGA port, an S-video port, a composite video port, an ethernet port, a firewire port, an eSATA port, a thunderbolt port, a DVI port, and a display port.

In some embodiments, the microfluidic device includes a plurality of sensors located in a number of microfluidic channels. Examples of sensors include impedance sensors capable of measuring an impedance value of a fluid including an analyte as the fluid is passed over the sensor. Other examples may include pH sensors and temperature sensors.

In some embodiments, the microfluidic device includes a plurality of resistors that serve as both microfluidic heaters and microfluidic pumps, depending on the amount of voltage applied to the resistor. Optionally, the resistors are thin film resistors. The thin film resistor may be made of tantalum or tantalum aluminum, platinum, gold, silicon carbide, silicon nitride, tungsten, or combinations thereof. In some embodiments, the thickness of the resistor may be approximately 500 angstroms to 5000 angstroms.

In some embodiments, the microfluidic device comprises an acoustic wave sensor configured such that the acoustic wave element is disposed on a principal surface of a piezoelectric substrate and a reactive membrane extends over the acoustic wave element. Optionally, the piezoelectric substrate is made of single-crystalline dielectric such as LiTaO3, LiNaO3, or quartz, for example. In some embodiments, the acoustic wave element includes comb-shaped IDT (interdigital transducer) electrodes arranged to excite a surface acoustic wave and also includes reflectors that are arranged on both sides of a region containing the IDT electrodes in the propagation direction of the surface acoustic wave. Optionally, the IDT electrodes and the reflectors are made of Al, Au, Pt, Cu, Ag, or an alloy containing these metals.

In some embodiments, the means for detecting the detectable label is a Field-Effect Transistor (FET). In some embodiments, the microfluidics device comprises a biosensor, such as a field-effect transistor-based sensor and a communication port. In some embodiments, the field-effect transistor-based sensor comprises a field effect transistor (FET) and a biological recognition element, such as a bio-sensitive layer, which may include a chelator or derivatized chelator. The FET includes a source, a drain, a gate, and a dielectric material, at least partially interposed between the source and the gate, and at least partially interposed between the drain and the gate. The biological recognition element, in some embodiments, may be at least partially disposed upon the dielectric material and/or at least partially disposed between the source and the drain. Optionally, the FET further comprises a carbon nanotube. In some embodiments, the carbon nanotube is at least partially disposed upon the dielectric material and/or at least partially disposed between the source and the drain, and the biological recognition element is at least partially disposed upon the carbon nanotube. The FET-based sensor optionally includes a sample portion configured to receive the detectable label and place the detectable label in contact with the biological recognition element. In some embodiments, the present disclosure provides a sample testing device comprising the FET-based sensor and a communication port. The sample testing device is configured to communicate, by way of the communication port, sensor data—which may be based on a signal provided by the drain and/or the source of the FET—to a computing device, such as a mobile computing device. In some embodiments, the present disclosure provides software that, when executed on the sample testing device and/or on the coupled computing device, provides a graphical user interface (GUI)—on the sample testing device and/or on the computing device—via which a user can interact with the sample testing device, for example by controlling sample tests, viewing sample test results, and/or the like. The FET-based sensor and/or the sample testing device, in various embodiments, may include a local power source, or may be powered by way of a remote power source, such as a power source included in the computing device, that may be coupled to a power port of the sample testing device and/or the FET-based sensor.

In some embodiments, the FET substrate comprises a dielectric material. Optionally, the substrate comprises Gallium Nitride.

In some embodiments, the FET comprises a chelator or a derivatized chelator. Optionally, the FET comprises a chelator or a derivatized chelator at least partially interposed between the source and the drain. In some embodiments, the chelator or the derivatized chelator is deposited on the surface of a scaffold within the microfluidics device (e.g., a FET within the microfluidics device) and is configured to contact a detectable label. Optionally, the detectable label comprises metal ion. The chelator or the derivatized chelator may be configured on the FET to selectively bind with the metal ion, such that the selective binding between the chelator or the derivatized chelator and the metal ion causes a change in an electrical current between the source and the drain. In some embodiments, the change in the electrical current is provided as output for use in at least one of detecting the metal ion, identifying the metal ion, or measuring an aspect of the metal ion. Optionally, a first electrical voltage is applied to the source and a second electrical voltage is applied to the drain, the first electrical voltage being different from the second electrical voltage, thereby contributing to the electrical current between the source and the drain.

Non-limiting examples of FET chips that can be used in the present disclosure are: a Gallium nitride (GaN) chip, a high quality Silicon Nanowire Field Effect Transistors (SiNW-FETs), a Metal Oxide Semiconductor Field Effect Transistor (MOSFET), a nanowire field-effect transistor (NWFET) chip, a carbon nanotube field-effect transistor (CNTFET) chip, an ion-sensitive field-effect transistor (ISFET) chip, an oxide-semiconductor field-effect transistor (OSFET) chip or a field-effect transistor chip fabricated by a complementary metal oxide semiconductor (CMOS) process. In some embodiments, the substrate comprises Gallium nitride. In some embodiments, the FETs have a semiconductor film (the channel) that is separated from an electrode (the gate) by a thin film insulator, made of e.g. silicon oxide, metal oxide or others. This gate-insulator-organic semiconductor sandwich is analogous to a capacitor that causes field-effect current modulation in the channel (between said source and drain electrodes which contact the semiconductor film). Hence, the current between the source and drain electrodes can be adjusted by tuning the voltage applied to the gate electrode.

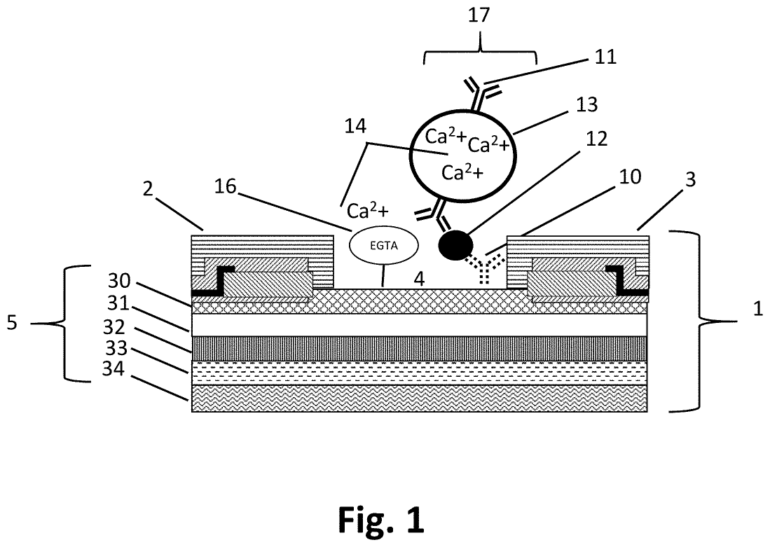

FIG. 1. shows a schematic side cross sectional representation of a transistor device (1) and liposome immunoassay in accordance with an aspect of the present disclosure. The detection is based on the release of calcium ions (Ca²⁺) (14) near the sensor-liquid-interface. The liposomes (13) containing Ca²⁺ are attached to the surface of a substrate (5) (comprised of layers 30, 31, 32, 33, and 34 in the example of FIG. 1), via an immunoassay consisting of an antibody conjugate (17) comprising a liposome (13) and a detection antibody (11), a target analyte (12), and capture antibody (10). A calcium chelator, such as EGTA (16), ethyleneglycol-bis(β-aminoethyl)-N,N,N′,N′-tetraacetic Acid binds Ca²⁺ ions near the FET gate (4). The transistor comprises a source (2) and a drain (3) deposited onto the substrate. The substrate consists of a layer of AlGaN (30), unintentionally doped (UID) GaN (31), Carbon Doped GaN (32), AN (33) and SiC (34).

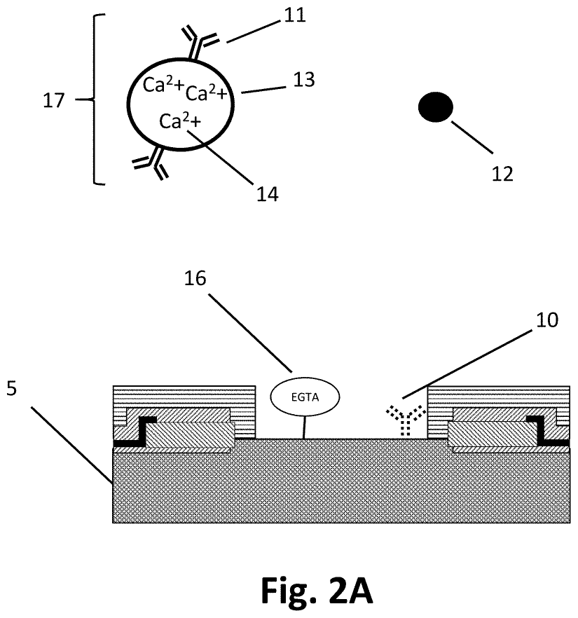

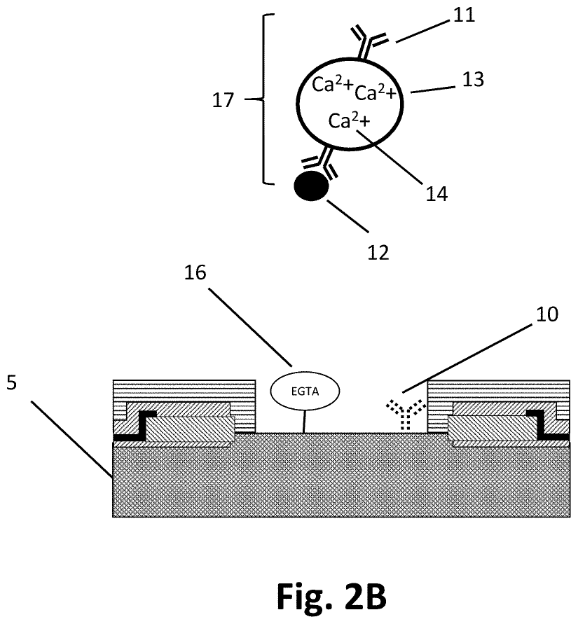

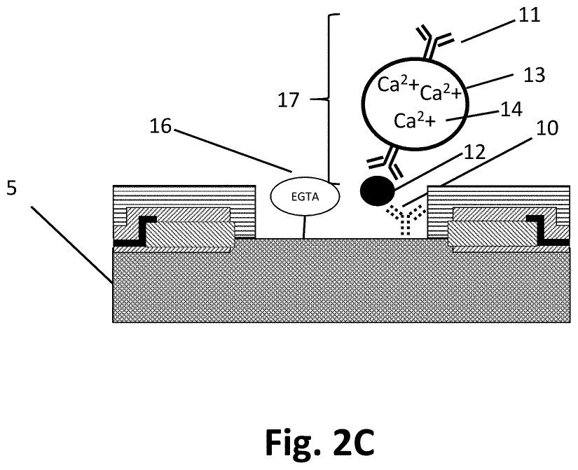

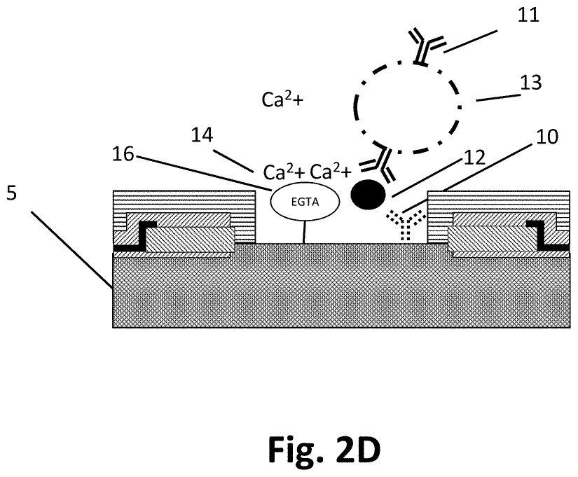

FIGS. 2 A-D show side cross sectional representations of a scheme for detection of a target analyte (12) in solution using FET transistors, such as transistor (1) described herein. FIG. 2A shows antibody conjugates (17) comprising liposomes (13) containing a solution of calcium ions (14) conjugated with detection antibodies (11), and target analytes (12) floating in a solution. The substrate (5) surface is functionalized with capture antibodies (10) and EGTA (16). FIG. 2B shows the antibody conjugate (17) and analyte (12) forming an immunoassay half sandwich-like structure in solution. FIG. 2C shows the formation of the immunoassay as the antibody conjugate (17) and analyte (12) bind to the capture antibody (10) on the surface of the substrate (5). FIG. 2D shows the release of the calcium ions (14) from the liposome (13) which rapidly bind with the EGTA (16) and create a detectable voltage change in the transistor (1).

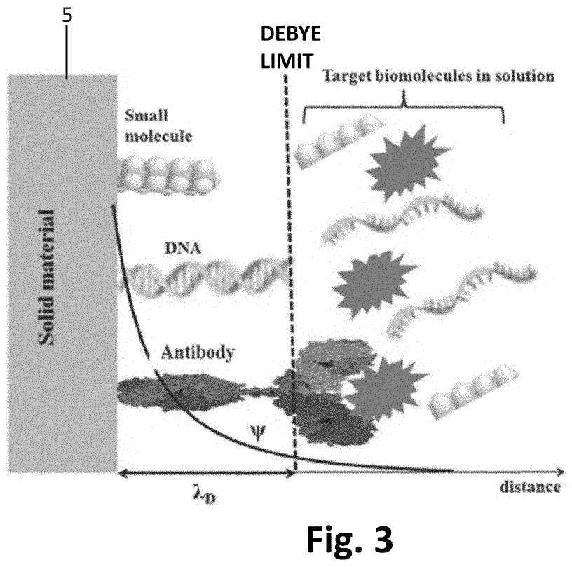

FIG. 3 shows the electrical double-layer length known as the Debye limit for materials ability to interact with a substrate interface 5 in order to make a detectable change in the device voltage.

In some embodiments, the amplification approach of the marker is based on the rapid release of metal ions near the sensor-liquid-interface. The capture antibody may be conjugated to the substrate surface of the FET. Optionally, the chelator or derivatized chelator is conjugated to the surface of the FET. Preferably, the capture antibody and the chelator or derivatized chelator are conjugated to the substrate surface of the FET device. In a second step, the detection antibody, linked to liposomes containing the metal ions (e.g. calcium ions), may selectively recognize the target marker, and the conjugate liposomes-antibody-marker may be put into contact with the capture antibody conjugated to the substrate surface. Alternatively, the capture antibody conjugated to the substrate surface may selectively recognize the target marker, and the detection antibody, linked to liposomes containing the metal ions (e.g. calcium ions) may be put into contact with the capture antibody-bound marker.

To bring metal ions near the surface of the channel or gate, the chelator or derivatized chelator may be conjugated to the substrate surface and binds metal ions near the FET gate.

If the marker has bound to the detection antibody and the capture antibody and metal ions have been released upon disruption of the liposomes, this may result in a detectable voltage shift associated with the change in current across the transistor due to the binding of metal ions to the chelator or derivatized chelator at the substrate surface, changing the transistor's electronic characteristics.

In some embodiments, the method or microfluidics device of the disclosure selectively detects a marker by detecting the ions released from the liposomes. In some embodiments, the microfluidics device comprises a FET detector. In any of the microfluidics device, FETs, sensors, or methods disclosed herein, the metal ion being detected is, optionally, selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion (e.g., As⁺³, Hg⁺², Sb⁺³, and Au⁺). Preferably, the metal ions to be detected are divalent and trivalent ions.

In some embodiments, the microfluidics device detects metal ions released from the liposomes. Optionally, the microfluidics device comprises a metal ion chelator or metal ion derivatized chelator which specifically binds the released ions. In some embodiments, the microfluidics device comprises a FET detector, which detects metal ions released from the liposomes. In some embodiments, the microfluidics device comprises a FET detector, which comprises a metal ion chelator or metal ion derivatized chelator which specifically binds the released ions.

In some embodiments, chelating agents of metallic ions include chelating agents of Ca²⁺, Fe²⁺, Fe³⁺, Mg²⁺, Mn²⁺, Cu²⁺, Cu³⁺, Zn²⁺, Ni²⁺, Co²⁺, heavy metal ions (e.g., As⁺³, Hg⁺², Sb⁺³, and Au⁺), and the like. The metal ion chelator or metal ion derivatized chelator may be any of the chelators described supra or derivatized therefrom. It is within the skill of the art to select a chelating agent or derivatized chelating agent that will bind or complex with a particular ion of interest. See, e.g., Bers D. M., MacLeod K. T. (1988) Calcium Chelators and Calcium Ionophores. In: Baker P. F. (eds) Calcium in Drug Actions. Handbook of Experimental Pharmacology, vol 83. Springer, Berlin, Heidelberg; Hatcher, H C. et al. Future Med Chem. 2009 December; 1(9): 10.4155; Sheth, S., Curr Opin Hematol 2014, 21:179; Missy

P. et al. Hum Exp Toxicol., 2000, vol. 19(8): 448-456; Sigma Aldrich, BioUltra Reagents: Chelators (available at https://www.sigmaaldrich.com/life-science/metabolomics/bioultra-reagents/chelators.html); Santa Cruz Biotechnology Chelators (available at https://www.scbt.com/scbt/browse/chelators/_/N-lazot5l); Lawson M K, et al. Curr Pharmacol Rep (2016) 2:271-280; Radford and Lippard,

Curr Opin Chem Biol. 2013 April; 17(2): 129-136; Chaitman, M. et al., P T. 2016 January; 41(1): 43-50, each of which is incorporated herein in its entirety.

The definitions and examples of chelating agents of metal ions are detailed above for the method of detection of a marker and apply herein for the microfluidics device.

In some embodiments, the marker detected by the microfluidics device or the method is a biomarker, an environmental marker, an allergen, or a microorganism. Optionally, the sample is an environmental sample, a food sample, or a sample obtained from a subject.

In some embodiments, the microfluidics device further comprises means for removing the marker not bound to the antibody. Optionally, the device comprises means for washing or filtering the antibody-bound marker.

There are several problems with conventional FETs for biological detection applications. In ionic solutions, the small ions, which carry an opposite charge to that of the detectable large macromolecule, screen the observed net charge by a cloud of opposite charge around the macromolecules. That screening is dependent on the distance between the surface (λ_{D}) and the point of observation (FIG. 3). At one Debye length, the charge effect on the voltage of the transistor decays 1/e, typically 1 nm. For large biomolecules such as antibodies, the area of interaction is much further out from the substrate and has very little effect on the detected voltage. For smaller DNA molecules, the interaction can occur at the boundary of the Debye limit. For small molecules, such as EGTA, the interaction falls within the Debye limit, allowing for a significant voltage shift to occur in the transistor. High ionic strength solutions pose difficulties for C-BioFETs since they are sensitive to protein charge and require a reference electrode. In a C-BioFET, the detection mechanism is based on the charge or potential of the target protein, which results in a potential change near the surface of the channel, thus causing a current change. This mechanism is constrained by the Debye length and the high ionic strength of the buffer required to maintain the conformation of the protein or biological sample. The signal amplifications described herein help overcome this limitation allowing a C-BioFET to deliver more charges to the channel

In a fifth aspect of the present disclosure, the microfluidics device as disclosed herein, can be used to detect more than one marker in a sample. Such a microfluidics device comprises:
(a) means for receiving a sample;
(b) a first capture molecule, wherein the first capture molecule is affixed to a first scaffold or is capable of binding to the first scaffold and binds a first marker in the sample;
(c) means for contacting the sample with the first capture molecule;
(d) a second capture molecule, wherein the second capture molecule is affixed to a second scaffold or is capable of binding to the second scaffold and binds a second marker in the sample, wherein the first marker is different from the second marker;
(e) means for contacting the sample with the second capture molecule;
(f) means for contacting the first marker with a composition comprising a first antibody conjugate, wherein the first antibody conjugate is an antibody conjugate according to the first aspect of the invention and binds to a different epitope of the first marker than the first capture molecule;
(g) means for contacting the second marker with a composition comprising a second antibody conjugate, wherein the second antibody conjugate is an antibody conjugate according to the first aspect of the invention and binds to a different epitope of the second marker than the second capture molecule;
(h) means for contacting the first marker-bound first antibody conjugate with conditions capable of releasing a first detectable label from the amphiphilic lipid vesicle on the first antibody conjugate;
(i) means for contacting the second marker-bound second antibody conjugate with conditions capable of releasing a second detectable label from the amphiphilic lipid vesicle on the second antibody conjugate;
(j) a first detector for the first detectable label; and
(k) a second detector the second detectable label.

In some embodiments, the microfluidics device to detect more than one marker in a sample comprises:
(a) means for receiving a sample;
(b) a first capture molecule, wherein the first capture molecule is affixed to a first scaffold or is capable of binding to the first scaffold and binds a first marker in the sample;
(c) means for contacting the sample with the first capture molecule;
(d) a second capture molecule, wherein the second capture molecule is affixed to a second scaffold or is capable of binding to the second scaffold and binds a second marker in the sample, wherein the first marker is different from the second marker;
(e) means for contacting the sample with the second capture molecule;
(f) means for contacting the first marker with a composition comprising a first aptamer conjugate, wherein the first aptamer conjugate is an aptamer conjugate according to the first aspect of the invention and binds to a different region of the first marker than the first capture molecule;
(g) means for contacting the second marker with a composition comprising a second aptamer conjugate, wherein the second aptamer conjugate is an aptamer conjugate according to the first aspect of the invention and binds to a different region of the second marker than the second capture molecule;
(h) means for contacting the first marker-bound first aptamer conjugate with conditions capable of releasing a first detectable label from the amphiphilic lipid vesicle on the first aptamer conjugate;
(i) means for contacting the second marker-bound second aptamer conjugate with conditions capable of releasing a second detectable label from the amphiphilic lipid vesicle on the second aptamer conjugate;
(j) a first detector for the first detectable label; and
(k) a second detector the second detectable label.

The skilled artisan would recognize that the antibody conjugate of the fifth aspect of the disclosure may be replaced with a mixture of aptamer conjugates and antibody conjugates.

A skilled person in the art will know how to adapt the microfluidics device in order to detect more than one marker in a sample. The embodiments of the fourth aspect of the disclosure apply herein to any of the embodiments for the microfluidics device to detect more than one marker or one of a plurality of markers in a sample.

Examples of means for receiving the sample, the capture molecule, means for contacting the sample with the capture molecule, means for contacting the capture molecule-marker with the antibody conjugate and means for contacting the antibody conjugate bound to the marker with a releasing composition can be found supra.

In some embodiments, the detectors for the first, second and any subsequent detectable labels are the same. Optionally, the first, second and any sequent detectable labels used in the microfluidics device are the same. In some embodiments, the detector for the first detectable label is different from the detector for the second detectable label. Optionally, the detector for any subsequent detectable label is different from the detector from the first detectable label, the detector for the second detectable label, and the detector for any other subsequent detectable label. The first detectable label may be different from the second detectable label. In some embodiments, any subsequent detectable label is different from the first detectable label, the second detectable label and any other subsequent detectable label.

In some embodiments, the first and second detectors are the same device. Optionally, the subsequent detector is the same device as the first and second detector. In some embodiments, the first detector is a different device from the second detector. Optionally, any subsequent detectors different devices than the first detector, the second detector and any other subsequent detector. In some embodiments, the first, second and any subsequent detectable labels can be detected by the same detector. Optionally, the microfluidics device further comprises one or more detectors for detecting the one or more additional detectable labels.

In some embodiments, the first and second scaffolds are the same. Optionally, any subsequent scaffolds are the same as the first and second scaffolds. In some embodiments, the first scaffold is different form the second scaffold. Optionally, any subsequent scaffolds are different from the first scaffold, the second scaffold, and any other subsequent scaffold.

In some embodiments, the first scaffold is the first detector. Optionally, the second scaffold is the second detector. In some embodiments, the first scaffold is adjacent to the first detector. Optionally, the second scaffold is adjacent to the second detector. In some embodiments, the microfluidics device further comprises means for transporting the first detectable label to the first detector. Optionally, the microfluidics device comprises means for transporting the second detectable label to the second detector.

In some embodiments, the first capture molecule is a capture antibody. Optionally, the first capture molecule is a capture aptamer. In some embodiments, the first capture molecule is bound to a magnetic bead or to a metallic bead. Optionally, the first capture molecule binds to the first scaffold upon the cycling of an electric current. In some embodiments, the second capture molecule is a capture antibody. Optionally, the second capture molecule is a capture aptamer. In some embodiments, the second capture molecule is bound to a magnetic bead or to a metallic bead. Optionally, the second capture molecule binds to the second scaffold upon the cycling of an electric current.

In some embodiments, the first antibody or aptamer conjugate is released from a first channel. Optionally, the second antibody or aptamer conjugate is released from a second channel. Any subsequent antibody or aptamer conjugate may be released from a subsequent channel. In some embodiments, the second antibody or aptamer conjugate is released from the second after the first antibody or aptamer conjugate is released from the first channel and the first detector detects the first detectable label. Optionally, any subsequent antibody or aptamer conjugate is released from the subsequent channel after the second antibody or aptamer conjugate is released from the second channel and the second detector detects the second detectable label.

In some embodiments, the first detector, the second detector, and/or any subsequent detector is a surface acoustic wave device. In such embodiments, the first and/or second detectable label is selected from the group consisting of a magnetic particle, a metal particle and a spore.

In some embodiments, the first detector, the second detector and/or any subsequent detector is a field effect transistor (FET). In such embodiments, the first and/or second detectable label is selected from the group consisting of a magnetic particle, a metal particle, and a charged solution. The charged solution may be an ionic solution. The FET may be a chelator-coated FET, such as those described in U.S. Provisional Application No. 62/718,632, U.S. Provisional Application No. 62/886,759 and PCT/US2019/046568, each of which is incorporated by reference herein in its entirety.

In some embodiments, the first detector, the second detector, and/or any subsequent detectors are selected from the group consisting of a fluorescent label detector, an enzymatic label detector, a radioactive label detector and a colorimetric label detector.

In some embodiments, the microfluidics device further comprises one or more additional capture molecules that bind to one or more additional scaffolds or are capable of binding one or more additional scaffolds and bind one or more additional markers in the sample, wherein the one or more additional markers a different from the first marker, the second marker and any other additional marker; and one or more additional antibody or aptamer conjugates comprising one or more additional detectable labels, wherein the one or more additional antibody or aptamer conjugates bind the one or more additional markers at different epitopes than the one or more capture molecules. In some embodiments, the one or more additional capture molecules comprise one or more capture antibodies. Optionally, the one or more additional capture molecules comprise one or more capture aptamers. In some embodiments, the one or more capture molecules comprise at least one capture antibody and at least one capture aptamer.

In some embodiments, the microfluidics device further comprises means for removing unbound first marker, unbound second marker and/or any unbound subsequent markers. Optionally, the microfluidics device further comprises means for washing the capture molecule which is bound to the first and/or second marker to remove the unbound markers. The microfluidics device may further comprise means for filtering the capture molecule which is bound to the first and/or second marker to remove the unbound markers. In some embodiments, the microfluidics device further comprises means for washing the capture molecule-bound one or more additional markers. Optionally, the microfluidics device further comprises means for filtering the capture molecule-bound one or more additional markers.

In some embodiments, the microfluidics device further comprises means for removing unbound first antibody or aptamer conjugate, unbound second antibody or aptamer conjugate and/or any unbound subsequent antibody or aptamer conjugates. Optionally, the microfluidics device further comprises means for washing the marker-bound first and/or second antibody or aptamer conjugate to remove the unbound antibody or aptamer conjugate. The microfluidics device may further comprise means for filtering the marker-bound first and/or second antibody or aptamer conjugate to remove the unbound antibody or aptamer conjugate. In some embodiment, the microfluidics device further comprises means for washing the marker-bound one or more additional antibody or aptamer conjugates. Optionally, the microfluidics device further comprises means for washing the marker-bound one or more additional antibody or aptamer conjugates.

In some embodiments, the microfluidics device comprises means for cycling an electric field or a magnetic field.

Optionally, the microfluidics device detects different types of markers, such as a biological marker, an environmental marker, an allergen or a microorganism. In some embodiments, the microorganism is a bacterium, an archaeon, an alga, a protozoan, a protist, a fungus or a virus.

In some embodiments, the composition capable of releasing the first, the second and/or any subsequent detectable labels comprises a detergent. Optionally, the detergent is a non-ionic detergent.

In some embodiments, the conditions or composition capable of releasing the first, the second and/or any subsequent detectable labels comprises an enzyme.

In some embodiments, the microfluidics device used to detect a marker in a sample comprises:
i. a primary chamber for containing a plurality of antibody conjugates in a buffer, each conjugated to a liposome containing a concentration of cations, in which the antibody-liposome conjugates specifically bind a selected marker;
ii. a testing chamber in which the marker-bound antibody conjugates in the buffer are immobilized by being attached to the surface by means of a plurality of capture antibodies;
iii. a secondary chamber for holding and selectively providing a washing buffer to the buffer in the testing chamber;
iv. a tertiary chamber for holding and selectively providing a liposome-disrupting solution to the buffer in the testing chamber;
v. means for performing convection enhanced delivery by recirculating a buffer including the marker-bound antibody conjugate multiple times through a fluidic circuit including the testing chamber to reduce time required to saturate the capture antibodies from diffusive timescales to convective timescales;
vi. a detector for measuring conductivity, impedance or resistivity of the buffer to determine the presence and/or extent of the binding of the marker to the antibody conjugate;
vii. an electrode isolated and covered with pure water as a continuous reference value subtracted from the measured conductivity, impedance or resistivity of the buffer to establish a delta value;
viii. a microcontroller for analysis of the delta value to generate a time coefficient (τ) for use in establishing reaction kinetics (k+/−);
ix. a first electrode disposed in the buffer, a second electrode disposed in the buffer to sense current according to a magnitude of cations released from the liposomes into the buffer, and a circuit for amplifying and/or signal conditioning the sensed current for output to the detector.

In some embodiments, the microfluidics device provides an improved limit of detection (LOD) and comprises:

a primary chamber for containing a plurality of antibody conjugates in a buffer, each having a liposome containing a concentration of ions or cations; in which the antibody-liposome conjugates specifically bind to a selected marker;

a testing chamber comprising a plurality of capture antibodies affixed to the surface of the testing chamber in which the marker-bound antibody conjugates in the buffer are immobilized;

a secondary chamber for holding and selectively providing a washing buffer to the buffer in the testing chamber;

a tertiary chamber for holding and selectively providing a liposome-disrupting solution to the buffer in the testing chamber; and

a detector for measuring conductivity, impedance or resistivity of the buffer to determine the presence and/or extent of conjugation of the analyte to the liposomes.

In some embodiments, the microfluidics device further comprises a sample port, such as a one-way fluidic port. Optionally, the sample port comprises a septum. In some embodiments, the sample port is configured to introduce a sample into the primary chamber. Optionally, the antibody conjugates bind a marker in the sample in the primary chamber, and the marker-bound antibody conjugate is transported to the testing chamber and the marker-bound antibody is immobilized by the capture antibodies. In some embodiments, the sample port is configured to introduce a sample into the testing chamber. Optionally, the capture antibodies bind a marker in the sample in the testing chamber and then antibody conjugate is transported to the testing chamber and binds the immobilized marker.

In some embodiments, the microfluidics device comprises a testing chamber which comprises a scaffold to which the marker-bound antibody conjugates are selectively attached. In some embodiments, the scaffold is an immobilized surface. In some embodiments, the marker-bound antibody conjugates are attached to a plurality of capture molecules (e.g., capture antibodies).

In some embodiments, the microfluidics device further comprises means for performing convection enhanced delivery by recirculating a buffer including the marker-bound antibody conjugates multiple times through a fluidic circuit including the testing chamber to reduce time required to saturate the capture molecules (e.g., capture antibodies) from diffusive timescales to convective timescales.

In some embodiments, the liposome-disrupting solution comprises a detergent. Optionally, the liposome-disrupting solution comprises a liposome-disrupting enzyme. Optionally, the enzyme is Phospholipase A2.

In some embodiments, the microfluidics device further comprises an electrode isolated and covered with pure water as a continuous reference value subtracted from the measured conductivity, impedance or resistivity of the buffer to establish a delta value.

In some embodiments, the microfluidics device further comprises a microcontroller for analysis of the delta value to generate a time coefficient (t) for use in establishing reaction kinetics (k+/−).

In some embodiments, the detector for measuring conductivity, impedance or resistivity of the buffer to determine the presence and/or extent of conjugation of the analyte to the liposomes further comprises a first electrode disposed in the buffer, a second electrode disposed in the buffer to sense current according to a magnitude of ions released from the analyte ELISA-type sandwich into the buffer, and a circuit for amplifying and/or signal conditioning the sensed current for output to the detector.

### EXAMPLES

The following examples are offered for illustrative purposes only and do not limit the scope of the present disclosure or paragraphs in any way. Indeed, various modifications of the disclosure in addition to those shown and described herein will become apparent to those skilled in the art from the foregoing description and fall within the scope of the paragraphs.

### Example 1: Antibody Conjugate Generation

A lipid vesicle or liposome with either one lipid, a mixture of lipids or a lipid polymeric membrane is assembled containing a high concentration (˜10⁶) of either ions or cations. The assembled liposomes are large unilamellar liposomes having a diameter of about ˜230 nm.

The liposome is formulated by using phospholipid with protein A/G incorporated in the structure, which will serve as an attachment site for the capture antibody. The mixed micelles of phosphatidylcholine (PTC) and octylglucoside (OG) leads to the formation of unilamellar phospholipid vesicles with a diameter of roughly 230 nm.

### Example 2: Design of a Microfluidics Device

An immobilizing surface of a detector is functionalized with a capture antibody in the testing reservoir of the microfluid circuit. This can be a flat surface, or a microfluidic channel network of functionalized pipes to increase the surface area of available binding sites. In addition to the testing reservoir, the device comprises three separate microfluidic chambers: primary, secondary and tertiary chambers; whereby the chemical sequencing events will be performed in the three microfluidic chambers to form an automatic process that reduces false positive results due to additional lipid vesicles from unbound antibody conjugates being suspended in the sample concentration. The manufactured antibody conjugates comprising thin layered vesicles containing a highly ionic fluid are disposed into the primary chamber of the multi reservoir microfluidic circuit. A wash buffer capable of eliminating any unbound antibody conjugates is disposed in the secondary chamber of the microfluidic circuit. A liposome-disruption solution, such as a detergent or a liposome-disrupting enzyme (e.g., Phospholipase A2 (PLA2)), or any solution that can disrupt the vesicle's lipid bilayer is disposed in the tertiary chamber. This will release the ions.

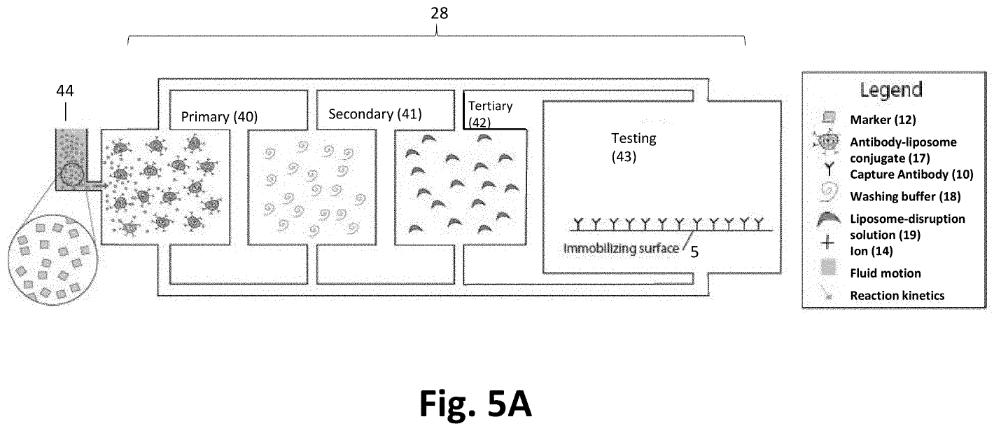

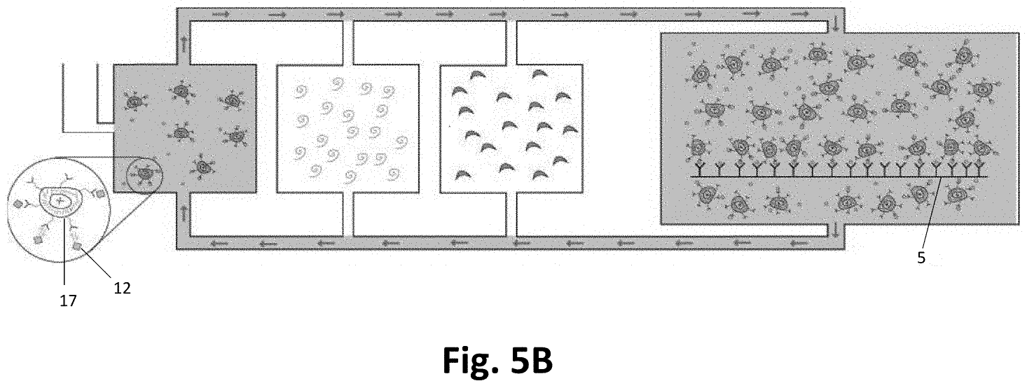

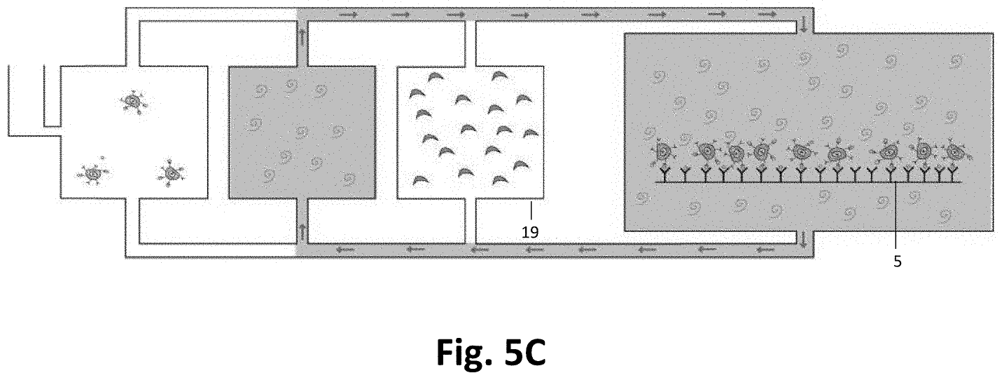

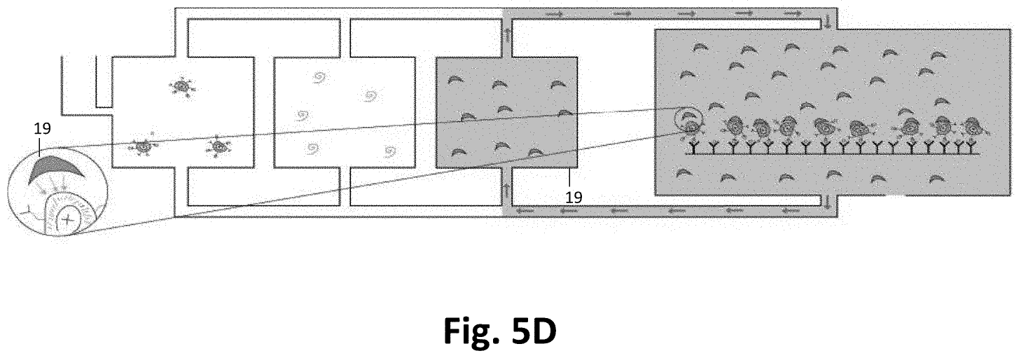

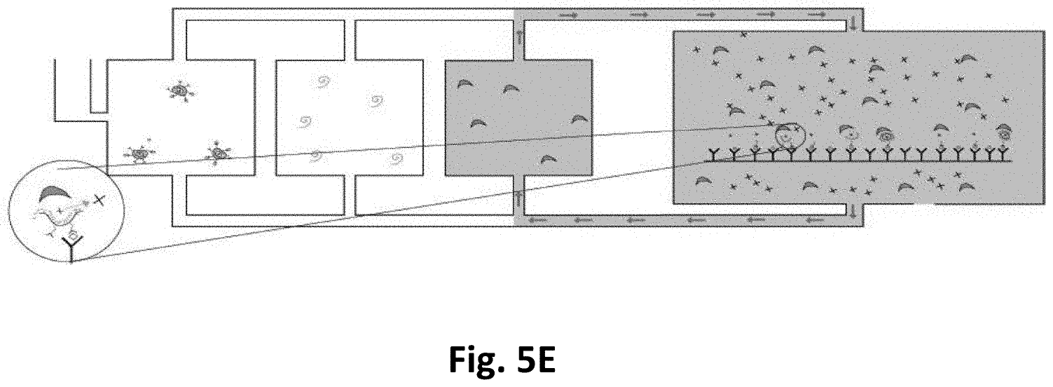

As shown in FIG. 5A, a sample comprising a marker (12) is introduced into a microfluidic cartridge 28 through a one-way fluidic port of the microfluidic device 44. This can be in the form of a septum or through any other means of introducing the sample to the primary chamber. The microfluidic device 28 will flow content from the primary chamber 40 into the testing reservoir 43. See FIG. 5B. Markers 12 will specifically bind to the detection antibodies 11 of the antibody-liposome conjugates 17 in the primary chamber 40. This first step begins the process of ultimately creating an ELISA-like sandwich comprising a functionalized surface 5, the marker 12, and an antibody-liposome conjugate 17, which comprises detectable ions 14.

As diagrammatically illustrated in FIG. 5B, the marker-bound antibody conjugate 17 is streamed through the microfluidic circuit to the functionalized surface 5 in the testing reservoir 43. The marker-bound antibody conjugates 17 bind to the capture antibodies 10 affixed to the immobilizing surface 5, creating an ELISA-like sandwich comprising the immobilizing surface 5 and maker-bound antibody-lipid vesicle conjugates 17.

Convection enhanced delivery (CED) can be utilized along the fluidic circuit to reduce the diffusive timescale for the marker-bound antibody conjugate to migrate to the immobilizing surface 5 and thus reduce the time required to saturate the capture antibodies from diffusive timescales to convective timescales. Typically, a pump and a recirculating line is included in the fluidic circuit to recirculate the buffer from the testing chamber through fluidic circuit for multiple passes within a predetermined time period, e.g. 50 recirculations within 10-15 minutes.

As shown in FIG. 5C, after sufficient time has passed and the capture antibodies 10 on the immobilizing surface 5 are deemed to be saturated with the sample comprising the marker-bound antibody conjugates 17 in the immobilizing surface 5, a solution comprising a wash buffer 18 will be released from the secondary chamber 41 in order to remove any unbound antibody conjugates 17 that have sedimented to the immobilizing surface 5 or any boundary such as a wall or floor of the testing reservoir 43. The wash buffer 18 will also have to remove any suspended concentration of antibody conjugates 17 from the fluidic circuit such that the only source of liposomes 13 in the testing reservoir 43 will be components of the immobilized marker-bound antibody conjugates 17. The suspended liposomes 13 can be removed from the microfluidic circuit before release of the liposome-disruption solution 19 (e.g., comprising a detergent or a lipid vesicle attacking enzyme) from the tertiary chamber 42 so that false positives from non-specific bound solvent lipid vesicles are not measured by the detector. In one embodiment of this process, an additional reservoir step will enable the wash buffer 18 to flush the entire fluidic content of the circuit.

The liposome-disruption solution 19 (e.g., comprising a detergent or a lipid vesicle attacking enzyme) from the tertiary chamber 42 is streamed into the testing reservoir 43 as diagrammatically depicted in FIG. 5D. The liposome-disruption solution 19 (e.g., comprising a detergent or a lipid vesicle attacking enzyme) will disrupt the liposomes 13 of the marker-bound antibody conjugates 17 and release the ions 14 contained therein as shown diagrammatically in FIGS. 5D and 5E. During the entire biochemical sequencing, the detector will record the impedance values (Z) or the conductivity of the liquid sample (mS/cm/s) while recording the established baseline value before and after ionic release.

In one possible configuration, the microfluidic device 28 will contain an electrode isolated and covered with pure water (18 MΩ) to have a continuous reference value which will be subtracted from the reading, to establish a delta value. The marker-bound antibody conjugates, having been disrupted by the liposome-disruption solution (e.g., comprising a detergent or a vesicle attacking enzymes 38), will release their concentrations of ions into the fluidic circuit. The impedance (or resistance, which might be faster to measure) will begin to climb as the fluid fills with ions. This electrical signal value will be sent to a microcontroller for analysis to generate the time coefficient (τ) for use in establishing the reaction kinetics (k^{+/-}).

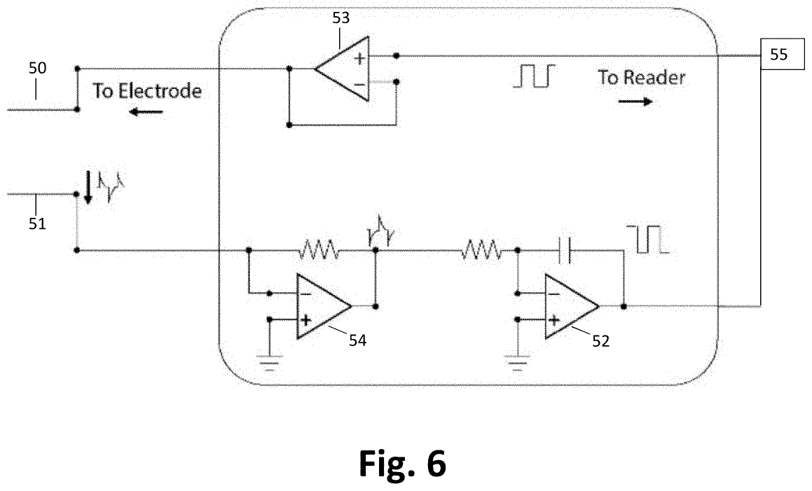

FIG. 6 is a schematic representation of one embodiment depicting an equivalent electrical circuit of detection electrodes 50 and 51, which are disposed in the buffer. The electrodes 50 forming the microfluidics device are interfaced with a driver circuit 53 and a capacitive detector circuit 52. The driver circuit 53 applies a square wave measuring signal to electrode 50. An op amp buffer 54 increases the input impedance of the detector circuit and ensures a near perfect square wave output from the non-square wave input signal. A current signal on electrode 51, which is proportional to the number of ions released from the liposomes or lipid vesicles of the previous assay is sensed by op amp buffer 54 and amplified by capacitive detector circuit 52 to provide an output of circuit proportional to conductivity, resistive or impedance value of the buffer. The active amplifier transforms the current signal into a voltage signal and is recorded using a microcontroller-controlled reader 55. The output of the galvanic cell formed out of the ion release into the buffer is continuously measured against a known reference, for example an electrode (not shown) placed within a pure water chamber (not shown) that will enable a differential output relative to the amount of ions released versus the reference steady state.

### Example 3: Detection of a Marker or Analyte Using a FET Device

The amplification approach of the analyte is based on the rapid release of calcium ions (Ca²⁺) near the sensor-liquid-interface. The capture antibody and the chelator (EGTA) were conjugated to the substrate surface. See FIGS. 2A-2D.

The detection antibody, linked to liposomes containing the calcium ions, selectively recognizes the target analyte. The conjugate liposomes-antibody-analyte were put into contact with the capture antibody conjugated to the substrate surface.

A wash step was performed to remove the free liposomes conjugated to detection antibodies, not specifically bound to the analyte.

A detergent (a non-ionic detergent, Triton X-100) was used to destabilize and disrupt the phospholipid bilayers of liposomes, so that the release of the calcium ions occurred.

To bring Ca²⁺ ions near the surface of the channel or gate, EGTA was conjugated to the substrate surface and bound Ca′ ions near the FET gate. See FIGS. 2A-2D.

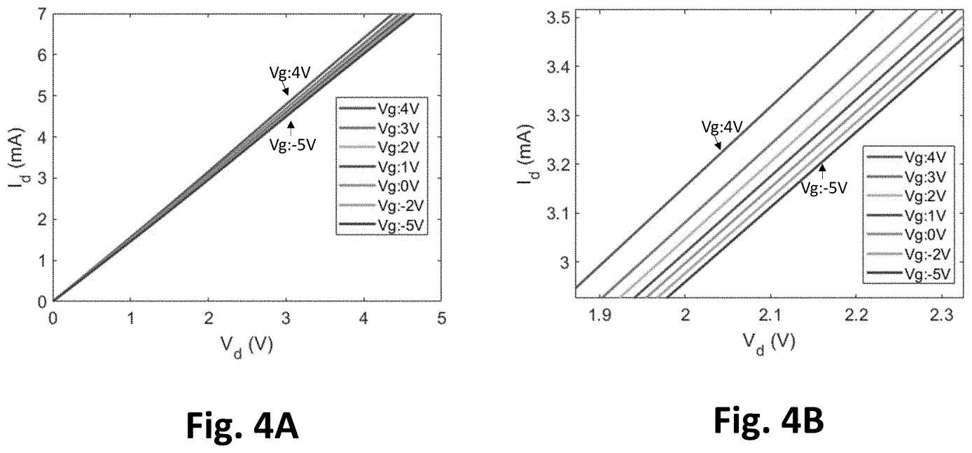

If the analyte has bound to the detection and capture antibodies and calcium ions have been released upon disruption of the liposomes, this results in a detectable voltage shift associated with the change in current across the transistor due to the binding of Ca′ at the substrate surface, changing the transistor's electronic characteristics. The change in current was measured using cyclic voltammetry, specifically, a potentiostat. In particular, the measurements confirmed a linear drain current dependence for the gate bias based on differing input voltage (−5V, −2V, 0V, 1V, 2V, 3V and 4V). See FIGS. 4A and 4B, where the graphs show I_{d} (drain current) as a function of V_{d} (drain voltage) of the measured dry I_{d}-V_{d} curves for the present FET transistor. As the gate bias is increased, the slopes of the linear portion of the I-V characteristics in FIG. 4A gradually increased as a result of the increasing conductivity of the channel.

1.-26. (canceled)

27. A method for detecting a marker in a sample by a detector generating a detection signal, the method comprising:
contacting the sample with a capture molecule that binds the marker, wherein the capture molecule is affixed to a scaffold or is capable of being affixed to a scaffold,
contacting the marker with a composition comprising the antibody or aptamer conjugate linked to an amphiphilic lipid vesicle including a detectable label, which antibody or aptamer conjugate includes an immobilization agent for immobilizing the marker near the detector; and means for amplifying the detection signal, where the immobilization agent of the marker binds a capture molecule to a scaffold, and where the means for amplification binds the immobilized marker with a detection molecule,
wherein the antibody or aptamer conjugate binds to a different epitope on the marker than the capture molecule;
contacting the marker-bound antibody or aptamer conjugate with conditions capable of releasing the detectable label from the amphiphilic lipid vesicle on the antibody or aptamer conjugate; and
detecting the detectable label.

28.-30. (canceled)

31. The method according to claim 27, wherein contacting the sample with a capture molecule that binds the marker, wherein the capture molecule is affixed to a scaffold or is capable of being affixed to a scaffold comprises affixing to a scaffold which is a detector for the detectable label.

32. The method according to claim 27, wherein contacting the sample with a capture molecule that binds the marker, wherein the capture molecule is affixed to a scaffold or is capable of being affixed to a scaffold comprises affixing to a scaffold adjacent to a detector for the detectable label.

33. The method according to claim 27, wherein the capture molecule is bound to a magnetic bead or a metallic bead, wherein contacting the sample with a capture molecule that binds the marker, wherein the capture molecule is affixed to a scaffold or is capable of being affixed to a scaffold comprises affixing to a capture molecule which binds to the scaffold upon the cycling of an electric current or a magnetic field.

34.-37. (canceled)

38. The method according to claim 37, wherein detecting the detectable label comprises using a detectable label is selected from the group consisting of a magnetic particle, a metal particle, a particle of 1 pg or greater, a spore, a charged particle, and an ionic solution or a combination thereof.

39.-42. (canceled)

43. The method according to claim 38, further comprising contacting a metal ion in the ionic solution with a metal ion chelator or metal ion derivatized chelator, wherein the metal ion chelator or metal ion derivatized chelator is located at or near a detector for the detectable label.

44.-51. (canceled)

52. A method of detecting one of a plurality of markers in a sample, the method comprising:
contacting the sample with a first capture molecule and a second capture molecule, wherein the first capture molecule is affixed to a first scaffold or is capable of being affixed to the first scaffold and binds a first marker,
wherein the second capture molecule is affixed to a second scaffold or is capable of being affixed to the second scaffold and binds a second marker, wherein the first marker is different from the second marker;
contacting the first marker with a composition comprising a first antibody or aptamer conjugate, wherein the first antibody or aptamer conjugate is an antibody or aptamer conjugate linked to a first amphiphilic lipid vesicle including a detectable label, which antibody or aptamer conjugate includes an immobilization agent for immobilizing the marker near the detector; and means for amplifying the detection signal, where the immobilization agent of the marker binds a capture molecule to a scaffold; and where the means for amplification binds the immobilized marker with a detection molecule, and wherein the first antibody or aptamer conjugate recognizes a different epitope on the first marker than the first capture molecule;
contacting the second marker with a composition comprising a second antibody or aptamer conjugate, wherein the second antibody or aptamer conjugate is an antibody or aptamer conjugate linked to a second amphiphilic lipid vesicle including a detectable label, which antibody or aptamer conjugate includes an immobilization agent for immobilizing the marker near the detector; and means for amplifying the detection signal, where the immobilization agent of the marker binds a capture molecule to a scaffold; and where the means for amplification binds the immobilized marker with a detection molecule, and wherein the second antibody or aptamer conjugate recognizes a different epitope on the second marker than the second capture molecule;
contacting the first marker-bound first antibody or aptamer conjugate with conditions capable of releasing a first detectable label from the first amphiphilic lipid vesicle on the first antibody or aptamer conjugate;
contacting the second marker-bound second antibody or aptamer conjugate with conditions capable of releasing a second detectable label from the second amphiphilic lipid vesicle on the second antibody or aptamer conjugate;
performing a first detection step to detect the first detectable label; and
performing a second detection step to detect the second detectable label.

53.-105. (canceled)

106. The method according to any one of claim 52, wherein the method further comprises:
contacting the sample with one or mare additional capture molecules, wherein each of the one or more additional capture molecules is attached to a scaffold or is capable of binding to a scaffold and binds a different marker than the first capture molecule, the second capture molecule and any other additional capture molecule;
contacting the one or more additional markers with a composition comprising one or more additional antibody conjugates, wherein each of the one or more additional antibody conjugates is an antibody conjugate, and wherein the one or more additional antibody conjugates recognize different epitopes on the different markers than the one or more capture molecules;
contacting the one or more marker-bound additional antibody conjugates with a composition capable of releasing one or more additional detectable labels from the amphiphilic lipid vesicle on the one or more additional antibody conjugates; and
performing one or more additional detection steps to detect the one or more additional detectable labels.

107.-112. (canceled)

113. A micro fluidics device comprising:
means for receiving a sample;
a capture molecule, wherein the capture molecule is affixed to a scaffold or is capable of binding to the scaffold and binds a marker in the sample;
means for contacting the sample with the capture molecule;
means for contacting the marker with a composition comprising an antibody or aptamer conjugate, wherein the antibody or aptamer conjugate is an antibody or aptamer conjugate linked to an amphiphilic lipid vesicle including a detectable label, which antibody or aptamer conjugate includes an immobilization agent for immobilizing the marker near the detector; and means for amplifying the detection signal where the immobilization agent of the marker binds a capture molecule to a scaffold; and where the means for amplification binds the immobilized marker with a detection molecule and binds to a different epitope of the marker than the capture molecule;
means for contacting the marker-bound antibody or aptamer conjugate with conditions capable of releasing a detectable label from the amphiphilic lipid vesicle on the antibody or aptamer conjugate; and
a detector for the detectable label.

114. The microfluidics device according to claim 113, wherein the scaffold is a detector for the detectable label.

115. The micro fluidics device according to claim 113, wherein the scaffold is adjacent to a detector for the detectable label.

116. The microfluidics device according to claim 113, wherein the capture molecule is bound to a magnetic bead or a metallic bead, wherein the capture molecule binds to the scaffold upon the cycling of an electric current.

117. The microfluidic device according to claim 113, wherein the device further comprises means for transporting the detectable label to the detector for the detectable label.

118. (canceled)

119. The micro fluidics device according to claim 113, wherein the detectable label is selected from the group consisting of a magnetic particle, a metal particle, a particle of 1 pg or greater, a charged particle, an ionic solution, and a spore or a combination thereof.

120.-123. (canceled)

124. The micro fluidics device according to claim 119, further comprising contacting a metal ion in the ionic solution with a metal ion chelator or metal ion derivatized chelator, wherein the metal ion chelator or metal ion derivatized chelator is located at or near the detector.

125.-136. (canceled)

137. A microfluidics device comprising
means for receiving a sample;
a first capture molecule, wherein the first capture molecule is affixed to a first scaffold or is capable of binding to the scaffold and binds a first marker in the sample;
means for contacting the sample with the first capture molecule;
a second capture molecule, wherein the second capture molecule is affixed to a second scaffold or is capable of binding to the scaffold and binds a second marker in the sample, wherein the first marker is different from the second marker;
means for contacting the sample with the second capture molecule;
means for contacting the first marker with a composition comprising a first antibody or aptamer conjugate, wherein the first antibody or aptamer conjugate is an antibody or aptamer conjugate linked to a first amphiphilic lipid vesicle including a detectable label, which antibody or aptamer conjugate includes an immobilization agent for immobilizing the marker near the detector; and means for amplifying the detection signal, where the immobilization agent of the marker binds a capture molecule to a scaffold; and where the means for amplification binds the immobilized marker with a detection molecule and binds to a different epitope of the first marker than the first capture molecule;
means for contacting the second marker with a composition comprising a second antibody or aptamer conjugate, wherein the second antibody or aptamer conjugate is an antibody or aptamer conjugate linked to a second amphiphilic lipid vesicle including a detectable label, which antibody or aptamer conjugate includes an immobilization agent for immobilizing the marker near the detector; and means for amplifying the detection signal, where the immobilization agent of the marker binds a capture molecule to a scaffold; and where the means for amplification binds the immobilized marker with a detection molecule and binds to a different epitope of the second marker than the second capture molecule;
means for contacting the first marker-bound first antibody or aptamer conjugate with a composition capable of releasing a first detectable label from a first amphiphilic lipid vesicle on the first antibody or aptamer conjugate;
means for contacting the second marker-bound second antibody or aptamer conjugate with a composition capable of releasing a second detectable label from a second amphiphilic lipid vesicle on the second antibody or aptamer conjugate;
a first detector for the first detectable label; and
a second detector the second detectable label.

138.-168. (canceled)

169. The microfluidics device according to claim 137, wherein the microfluidics device further comprises one or more additional capture molecules that bind to one or more additional scaffolds or are capable of binding one or more additional scaffolds and bind one or more additional markers in the sample, wherein the one or more additional markers a different from the first marker, the second marker and any other additional marker; and one or more additional antibody conjugates comprising one or more additional detectable labels, wherein the one or more additional antibody conjugates bind the one or more additional markers at different epitopes than the one or more capture molecules.

170.-176. (canceled)

177. The microfluidics device according to claim 169, wherein the device further comprises means for washing the capture molecule-bound one or more additional markers.

178. The microfluidics device according to claim 169, wherein the device further comprises means for washing the marker-bound one or more additional antibody conjugates.

179-192. (canceled)

193. An antibody or aptamer conjugate for the detection of a marker by a detector generating a detection signal comprising:
an immobilization agent for immobilizing the marker near the detector; and
means for amplifying the detection signal,
where the immobilization agent of the marker binds a capture molecule to a scaffold; and where the means for amplification binds the immobilized marker with a detection molecule.
